(19) United States
(12) Patent Application Publication
(10) Pub. No.: US 2025/0206782 A1
(43) Pub. Date: Jun. 26, 2025

(54) BIOLOGICAL POLYMERS

(71) Applicant: UNITED KINGDOM RESEARCH AND INNOVATION, Swindon (GB)

(72) Inventors: Martin SPINCK, Swindon (GB); Carlos PIEDRAFITA, Swindon (GB); Wesley E. ROBERTSON, Swindon (GB); Jason W. CHIN, Swindon (GB)

(21) Appl. No.: 18/987,121

(22) Filed: Dec. 19, 2024

Related U.S. Application Data
(60) Provisional application No. 63/613,258, filed on Dec. 21, 2023.

(51) Int. Cl. C07K 11/02; C12N 9/00; C12N 15/113

(52) U.S. Cl. CPC C07K 11/02; C12N 9/93; C12N 15/113

(57) ABSTRACT

The present invention relates to a prokaryotic cell, wherein: at least one sense codon is reassigned to a first orthogonal tRNA synthase/tRNA pair; at least one nonsense codon which is reassigned to a second orthogonal tRNA synthase/tRNA pair; the first and second orthogonal tRNA synthase/tRNA pair are further mutually orthogonal; both the first and second orthogonal tRNA synthase/tRNA pairs preferentially recognise non-canonical, non-alpha-amino acids, and to method using such cells to produce polymers comprising non-alpha-amino acids.

### SEQUENCE LISTING

The instant application contains a Sequence Listing which has been submitted electronically in XML format and is hereby incorporated by reference in its entirety. Said XML copy, created on Mar. 3, 2025 is named 51689-012002_Sequence_Listing_3_3_25 and is 114,037 bytes in size.

### FIELD OF THE INVENTION

The present invention relates to novel prokaryotic cells for the production of polymers containing non-canonical, non-alpha-amino acids, and to methods for making said cells. The invention also relates to newly obtainable polymers as produced by the prokaryotic cells of the invention. In addition, the invention relates to new orthogonal aminoacyl-tRNA synthetases (aaRSs) and orthogonal tRNAs, which may be used in pairs and find utility in host cells such as, but not limited to, the prokaryotic cells of the invention.

### BACKGROUND OF THE INVENTION

Nature uses 64 triplet codons to encode the synthesis of proteins composed of the twenty canonical amino acids, and most amino acids are encoded by more than one synonymous codon. It is widely hypothesized that removing sense codons and the tRNAs that read them from the genome may enable the creation of cells with several properties not found in natural biology—including new modes of viral resistance and the ability to encode the biosynthesis of non-canonical heteropolymers (3-6).

Current strategies for encoding new monomers in cells are limited to encoding a single type of monomer (commonly in response to the amber stop codon) (3, 10, 11), inefficient, or incompatible with encoding sequential monomers (12-17); these limitations preclude the synthesis of non-canonical heteropolymer sequences composed entirely of non-canonical monomers.

Some of the current platforms for the synthesis of polymers containing a non-canonical amino acid make use of an orthogonal aaRS/tRNA pair. Such pairs may be used to insert the non-canonical amino acid during protein synthesis. These pairs must be further engineered to decode a distinct target codon and to use a unique monomer that is not a substrate for other aaRSs.

Nature synthesizes peptide and depsipeptide macrocycles—including many antibiotics, immunosuppressants and anti-tumor compounds—containing an array of non-canonical and non-amino acid monomers. These molecules are synthesized using either: i) megadalton protein complexes (non-ribosomal peptide synthetases, NRPSs) or ii) post-translational modification of the canonical amino acids following ribosomal polymerization (ribosomally synthesized and post-translationally modified peptides, RiPPs). Despite substantial effort over several decades, and notable successes (42-47), the ability to engineer these systems to predictably generate any desired product remains largely elusive.

In vitro translation has been used to encode diverse macrocycles (48), and proteins and peptides incorporating alpha hydroxy acids (49, 50), and the cellular synthesis of macrocyclic peptides composed of the canonical amino acids has been realised (51).

Recently a strain of E. coli, Syn61, was created with a synthetic recoded genome in which all annotated occurrences of two sense codons (serine codons TCG and TCA) and a stop codon (TAG) were replaced with synonymous codons (18). This strain grows 1.6-fold slower than the strain from which it was derived.

Following further directed evolution of Syn61 to delete the tRNAs that decode TCG and TCA codons and the release factor (RF-1) that terminates protein synthesis in response to the amber stop codon a further developed strain (Syn61Δ3) and its evolved derivatives, including Syn61Δ3(ev5), in which two tRNAs and RF-1 were deleted, was created.

We showed that we could introduce the codons no longer present in the genome into synthetic genes and reassign these codons, using mutually orthogonal engineered aminoacyl-tRNA synthetase/tRNA pairs, to non-canonical amino acids (ncAAs); this enabled the synthesis of proteins containing several ncAAs, the encoded cellular synthesis of polymers composed entirely of ncAAs, and the encoded synthesis of a single five membered macrocycle containing ncAAs (52).

However, the genetically encoded polymerization of alpha-hydroxy acids to create macrocyclic depsipeptides (or the cellular polymerization of any non-alpha-L-amino acid linked monomer to synthesize a macrocycle), via cell-based translation, has not, to the best of our knowledge, been addressed.

### SUMMARY OF THE INVENTION

In a first aspect, there is provided a prokaryotic cell wherein:
a. at least one sense codon is reassigned to a first orthogonal tRNA synthase/tRNA pair;
b. at least one sense or nonsense codon which is reassigned to a second orthogonal tRNA synthase/tRNA pair;
c. the first and second orthogonal tRNA synthase/tRNA pair are further mutually orthogonal;
d. both the first and second orthogonal tRNA synthase/tRNA pairs preferentially recognise non-canonical, non-alpha-amino acids.

More than one sense or nonsense codon can be reassigned. Thus, in one embodiment, a second sense codon is reassigned to a third orthogonal tRNA synthase/tRNA pair which preferentially recognises a non-alpha amino acid, said pair being further orthogonal to the first and second tRNA synthase/tRNA pairs.

In the context of the present invention, a non-alpha-amino acid is an acid which can be recognised by a tRNA and attached thereto by an amino-acyl tRNA synthase (aaRS), and incorporated into a polymer by a ribosome, but which is not an amino acid in that it lacks an alpha amino group. Naturally occurring aaRS habitually only accommodate amino acids, so a modified aaRS is preferred to assemble non-amino acids, as described below.

Suitably, the non-alpha-amino acid is an alpha-hydroxy acid. Such acids are incorporated into polymers to form an ester bond, and thus potentially a polyester, rather than a peptide. Depsipeptides typically comprise a mixture of peptide and ester bonds.

The prokaryotic cell according to the invention is modified to remove occurrences of the reassigned codons. Accordingly, in one aspect the present invention employs a prokaryotic cell comprising 5 or fewer occurrences of one or more sense codons. In some embodiments the cell comprises 4 or fewer, 3 or fewer, 2 or fewer, 1 or fewer, or no occurrences of one or more sense codons. In some embodiments the one or more sense codons consist of one sense codon or two sense codons, preferably two sense codons. In some embodiments the cell comprises no occurrences of two or more sense codons, preferably two sense codons, and no occurrences of one stop codon, preferably the amber stop codon (TAG).

The prokaryotic cell may be a bacterial cell, preferably an Escherichia coli, Salmonella enterica, or Shigella dysenteriae cell. The prokaryotic cell may be viable.

In some embodiments the one or more sense codons are selected from TCG, TCA, TCT, TCC, AGT, AGC, GCG, GCA, GCT, GCC, CTG, CTA, CTT, CTC, TTG, and TTA, preferably the one or more sense codons are selected from TCG, TCA, AGT, AGC, GCG, GCA, CTG, CTA, TTG, and TTA, more preferably the one or more sense codons are selected from TCG, TCA, AGT, AGC, TTG, TTA, GCG and GCA, most preferably the one or more sense codons are TCG and/or TCA.

In some embodiments the synthetic prokaryotic genome comprises 10 or fewer, 5 or fewer, or no occurrences of the amber stop codon (TAG).

In a further aspect the prokaryotic cell comprises less than 10%, 5%, 2%, 1%, 0.5%, 0.1% of the occurrences of one or more sense codons, relative to a parent unmodified prokaryotic cell, or wherein the prokaryotic cell comprises no occurrences of one or more sense codons. In some embodiments the one or more sense codons consist of one sense codon or two sense codons, preferably two sense codons.

In some embodiments the one or more sense codons are selected from TCG, TCA, TCT, TCC, AGT, AGC, GCG, GCA, GCT, GCC, CTG, CTA, CTT, CTC, TTG, and TTA, preferably the one or more sense codons are selected from TCG, TCA, AGT, AGC, GCG, GCA, CTG, CTA, TTG, and TTA, more preferably the one or more sense codons are selected from TCG, TCA, AGT, AGC, TTG, TTA, GCG and GCA, most preferably the one or more sense codons are TCG and/or TCA.

The occurrences of the one or more sense codons in the genes may be replaced with synonymous sense codons, preferably TCG codons are replaced with AGC and/or TCA codons are replaced with AGT.

In embodiments, sense codon reassignment involves the replacement of substantially all identical sense codons on essential genes of the bacterial cell.

The essential genes may comprise essential genes selected from one or more of the list consisting of: ribF, IspA, ispH, dapB, folA, imp, yabQ, ftsL, ftsl, murE, murF, mraY, murD, ftsW, murG, murC, ftsQ, ftsA, ftsZ, IpxC, secM, secA, can, folK, hemL, yadR, dapD, map, rpsB, tsf, pyrH, frr, dxr, ispU, cdsA, yaeL, yaeT, IpxD, fabZ, IpxA, IpxB, dnaE, accA, tilS, proS, yafF, hemB, secD, secF, ribD, ribE, thiL, dxs, ispA, dnaX, adk, hemH, IpxH, cysS, folD, entD, mrdB, mrdA, nadD, holA, rlpB, leuS, Int, ginS, fldA, cydA, infA, cydC, ftsK, lolA, serS, rpsA, msbA, IpxK, kdsB, mukF, mukE, mukB, asnS, fabA, mviN, me, fabD, fabG, acpP, tmk, holB, lolC, lolD, lolE, purB, minE, minD, pth, prsA, ispE, lolB, hemA, prfA, prmC, kdsA, topA, ribA, fabl, tyrS, ribC, ydiL, pheT, pheS, rplT, infC, thrS, nadE, gapA, yeaZ, aspS, argS, pgsA, yefM, metG, folE, yejM, gyrA, nrdA, nrdB, folC, accD, fabB, gItX, ligA, zipA, dapE, dapA, der, hisS, ispG, suhB, tadA, acpS, era, rnc, IepB, rpoE, pssA, yfiO, rpIS, trmD, rpsP, ffh, grpE, csrA, ispF, ispD, ftsB, eno, pyrG, chpR, Igt, fbaA, pgk, yqgD, metK, yqgF, plsC, ygiT, parE, ribB, cca, ygjD, tdcF, yraL, yhbV, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, secY, rplO, rpmD, rpsE, rplR, rplF, rpsH, rpsN, rplE, rplX, rplN, rpsQ, rpmC, rplP, rpsC, rpIV, rpsS, rplB, rplW, rplD, rplC, rpsJ, fusA, rpsG, rpsL, trpS, yrfF, asd, rpoH, ftsX, ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, glmS, glmU, wzyE, hemD, hemC, yigP, ubiB, ubiD, hemG, yihA, ftsN, muri, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, and dnaC.

The non-alpha-amino acids according to the invention are advantageously alpha-hydroxy acids, which have the general structure RCH(OH) COOH, as opposed to an amino acid which has the general structure RCH(NH₂)COOH.

The reassigned codons may also be used to incorporate amino acids into the polymer, for example non-canonical amino acids which would not be incorporated according to natural codon assignment. The encoded polymer would therefore comprise both peptide and ester bonds.

The polymer may be a cyclic polymer, for example a macrocycle, which may comprise ester and/or peptide bonds, as above. Macrocycles can be cyclised post-expression, for example by expression in fusion with inteins or other elements that promote cyclization. Suitable systems are known in the art, and discussed further herein. Exemplary cyclic polymers are depsipeptides.

In one example, the polymer can be expressed as a fusion protein, wherein a nucleic acid encodes:
a. an N-terminal small ubiquitin-like modifier (SUMO) element, followed by a cysteine;
b. the sequence of a depsipeptide macrocycle, said sequence comprising two or more codons which are recognised by two or more orthogonal RNA synthase/tRNA pairs, said pairs preferentially recognising an alpha-hydroxy acid, said codons being selected from TCA, TCG and TAG;
c. A GyrA intein-CBD fusion element C-terminal to the sequence in (b).

In another aspect, there is provided a method of making a cell according to the prior aspect of the invention.

In one embodiment, the method the method comprises:
(i) modifying a prokaryotic cell to express a first orthogonal tRNA synthetase—tRNA pair suitable for decoding a first type of sense codon, and a second orthogonal tRNA synthetase—tRNA pair suitable for decoding a second type of sense codon wherein the prokaryotic cell comprises a genome wherein the first and second types of sense codon have been recoded such that a first and second endogenous tRNA are dispensable;
(ii) incubating the prokaryotic cell in the presence of first and second non-alpha-amino acids which are substrates for the first and second orthogonal tRNA synthetases; and
(iii) modifying the endogenous genes encoding the first and second endogenous tRNAs such that the first and second endogenous tRNAs are not expressed.

A third orthogonal tRNA synthetase/tRNA pair may be used, to target a third reassigned codon; and so on.

Orthogonal tRNA synthetase/tRNA pairs can be sourced from a number of organisms, and typically are derived from archaebacteria.

In embodiments, the orthogonal tRNA synthetase is modified by mutagenesis at at least one position in its amino acid sequence. Mutations can be introduced to improve the ability to the synthetase to incorporate desired monomers, such as non-alpha-amino acids, for example alpha-hydroxy acids. Preferred locations for the introduction of mutations include M300, L301, A302, M344 and N346 of MmPylRS, or equivalents in other aaRS.

The invention moreover provides a nucleic acid encoding a polymer which can be expressed in a cell according to the prior aspects of the invention, to produce a polymer having one or more non-peptide bonds, such as an ester bond, though incorporation of a non-alpha-amino acid.

Also provided is a method for producing a depsipeptide macrocycle, comprising expressing a nucleic acid according to the previous aspect in a prokaryotic cell as described herein such that alpha-hydroxy monomers are incorporated into the polymer encoded by the nucleic acid.

In embodiments, the first and second aaRS/tRNA pairs are selected such that their monomer incorporation preferences are specific for different alpha-hydroxy acid monomers.

Selection of reassigned codons can involve determining the monomer incorporation preference for a plurality of aaRS/tRNA pairs, and selecting a reassignment scheme for codons in the nucleic acid which assigns aaRS/tRNA pairs according to the desired monomer incorporation pattern or sequence.

An important aspect of the present invention is the provision of a modified pyrrolysyl or tyrolysyl amino acid tRNA synthetase (aaRS) and paired tRNA which preferentially bind to alpha-hydroxy acids, optionally with an aromatic side-chain. A modified aaRS can be produced by mutagenesis and selection as described herein.

Although the use of pyrrolysyl-tRNA synthetases from Methanocarcina mazei (Mm) and M. barkeri (Mb) to incorporate alpha-hydroxy acids has previously been shown at single sites in peptide, the efficiency and specificity of such incorporation has been poor. In order to improve the efficiency of alpha-hydroxy acid incorporation, an aaRS may be modified by: constructing a library of aaRS genes, using degenerate codons in the active site of the enzyme proximal to the location of the alpha-amino group of a bound amino acid; screening the library in the presence of the target alpha-hydroxy acid; identifying clones which incorporate alpha-hydroxy acids, and subjecting said clones to negative screening against alpha-amino acids to select against incorporation of amino acids.

In embodiments, the clones can be subjected to further mutagenesis in the region of the binding pocket which receives the monomers, and screened to identify further clones which are capable of binding other alpha-hydroxy acids, using the same positive and negative selection protocol.

Once aaRS specific for desired non-alpha-amino acids have been identified, there is provided a method for selecting mutually orthogonal aaRS/tRNA pairs for incorporation of multiple non-alpha-amino acids comprising screening pairs of aaRS/tRNA combinations against a panel of non-alpha-amino acids and identifying those combinations which selectively incorporate one member of the panel; and subsequently designing a reassignment scheme which associates aaRS/tRNA pairs with specific non-alpha-amino acids, and allows selection of appropriate aaRS/tRNA pairs depending on the desired non-alpha-amino acids to be incorporated.

### BRIEF DESCRIPTION OF THE FIGURES

FIG. 1. Genetically programmed cell-based synthesis of non-natural macrocycles.

a, Macrocycles and the genes that program their cell-based synthesis; macrocycle sequences are inspired by the natural sequences of SanA1-4 and YM (FIG. 2). The TCG codon (blue) programs the incorporation of non-canonical monomer A (blue star), and the TAG codon (green) programs the incorporation of non-canonical monomer B (green star). Macrocycle sequences are expressed as SUMO/intein (gyrA) fusions in Syn61Δ3(ev5) and cyclized by addition of Ulp1 (a C-terminal SUMO protease) and reductant13. Leu, Val, Cys, Ala, Thr, grey spheres, are canonical amino acids (Leucine, Valine, Cysteine, Alanine and Threonine, respectively).

b, Reassignment schemes 1-3 (r.s. 1-3) used for macrocyclic peptide synthesis. The reassignment schemes define the identity of monomers A and B; monomer A (blue) is incorporated in response to the TCG codon and monomer B (green) is incorporated in response to the TAG codon.

c-d, Mass spectra and structures of purified SanA1BA r.s. 3 and YMBA r.s. 1. The asterisk* denotes that the azide of pAzF is reduced to an amine, as previously reported16. The sequence synthesized is defined by the macrocycle sequence (defined in panel a) and the reassignment scheme (defined in panel b); thus, SanA1BA r.s. 3 defines the SanA1BA sequence in which the identity of A and B are defined by reassignment scheme 3.

e, Chemical structure of all cyclic peptides, indicating the positions of non-canonical amino acids (ncAAs) A and B. For each reassignment scheme (r.s. 1-3), ‘yes’ indicates that we detected the exact mass of the peptide following its purification, and ‘no’ indicates that we could not detect the peptide; see FIG. 4 for the raw spectra and structure of the confirmed cyclic peptides. We were unable to express the fusion of SanA3AB or BA with r.s. 2, and we were unable to extract the peptides generated from SanA4AB or BA using r.s. 2 or from SanA3AB with r.s. 1. The cyclic peptide precursors were purified by Ni-NTA and the peptide excised using Ulp1 and reductant.

FIG. 2. Chemical structure of Sansalvamide A and YM-254890

a-b, Chemical structure Sansalvamide A (panel a) and YM-254890 (panel b) with modification of the backbone highlighted in red and non-natural sidechains coloured blue. The base sequence was derived from both peptides omitting all modifications.

FIG. 3. SUMO-peptide-GyrA expressions and yields.

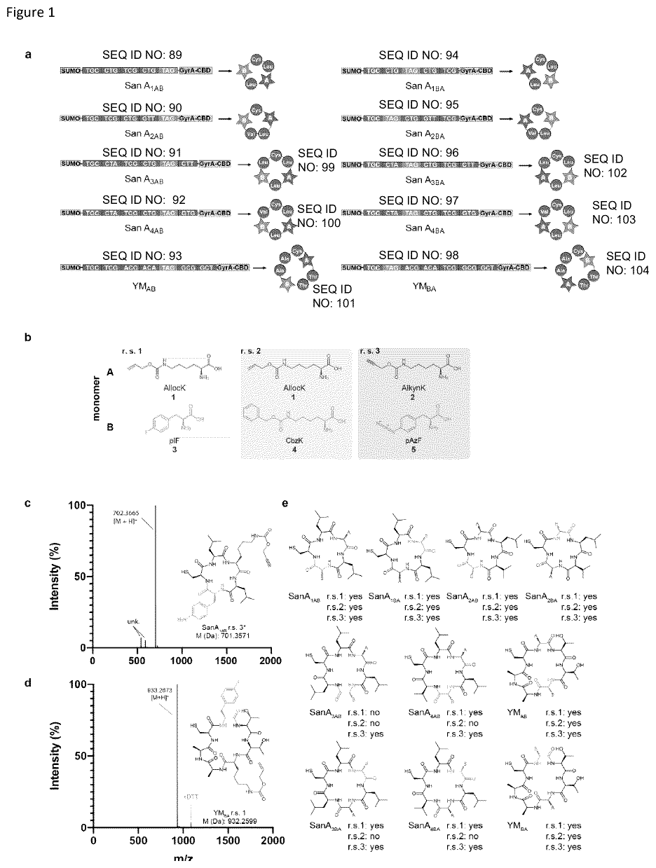

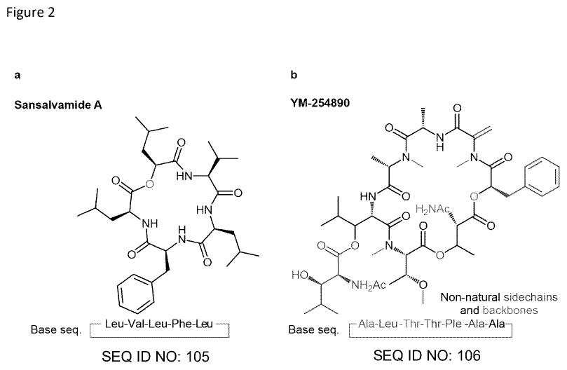

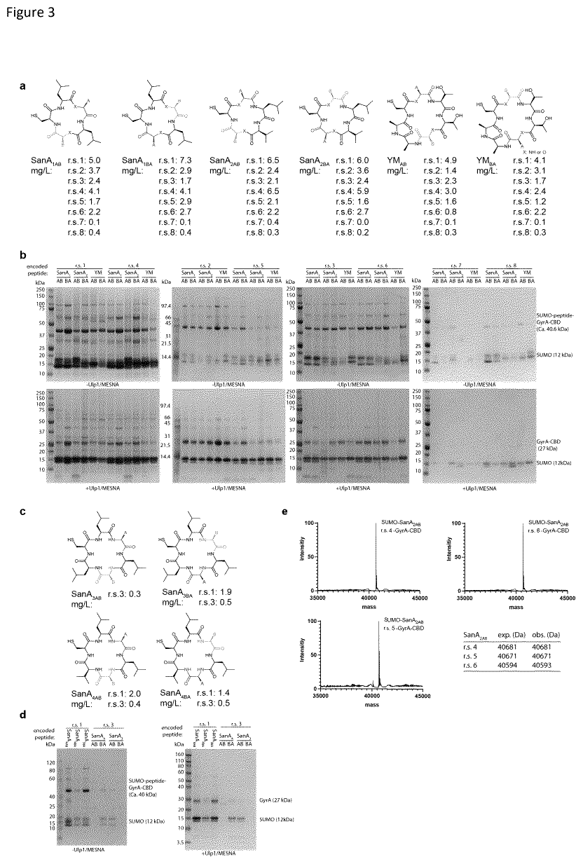

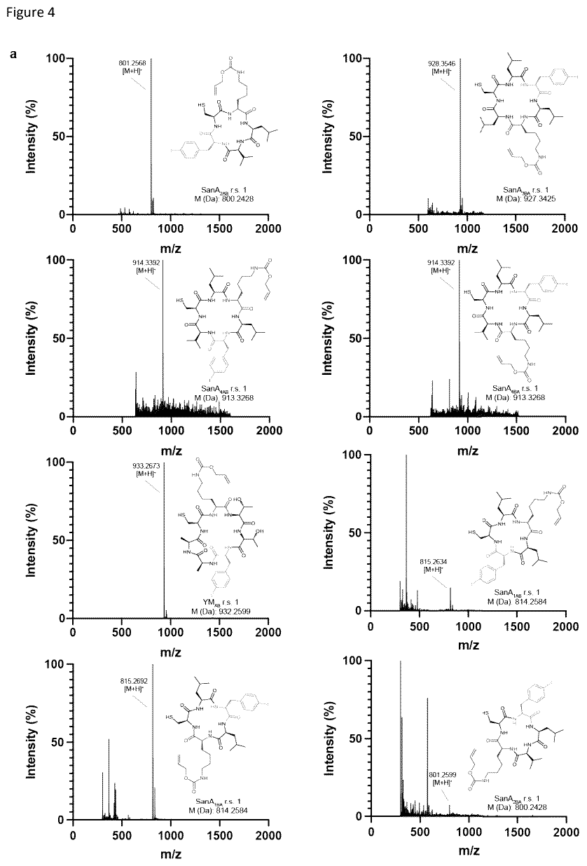

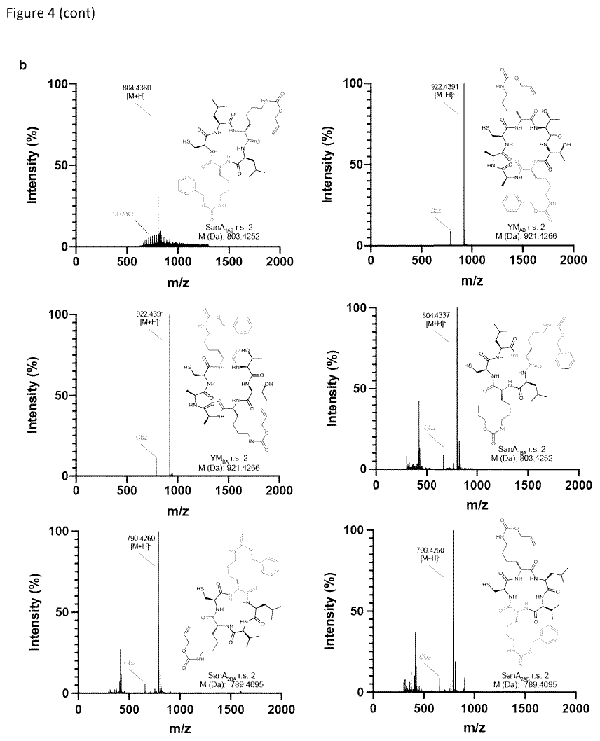

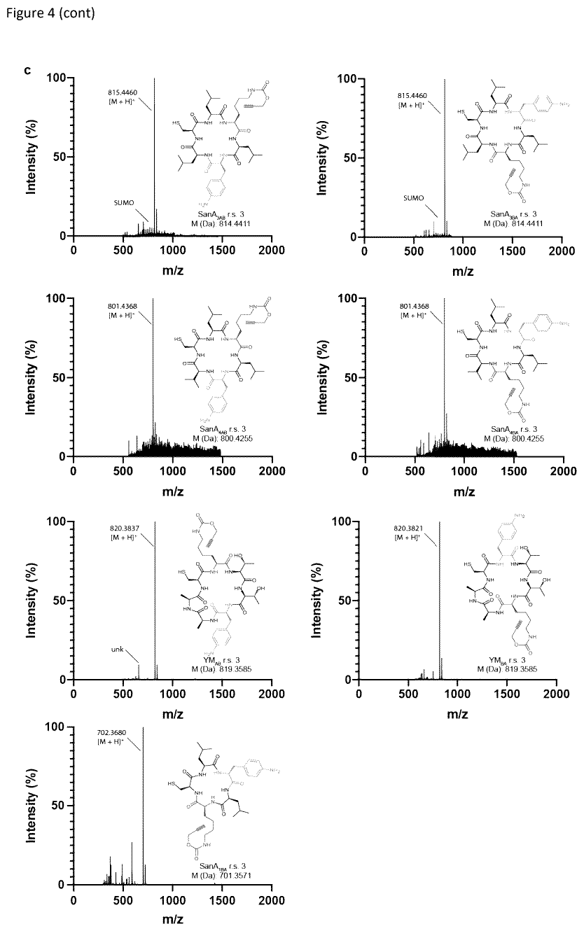

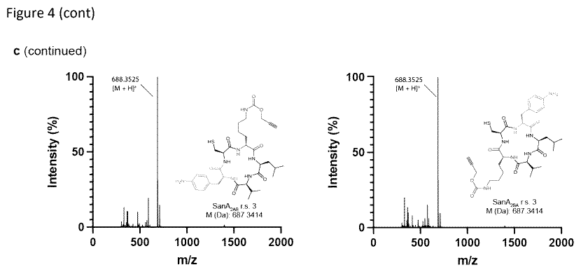

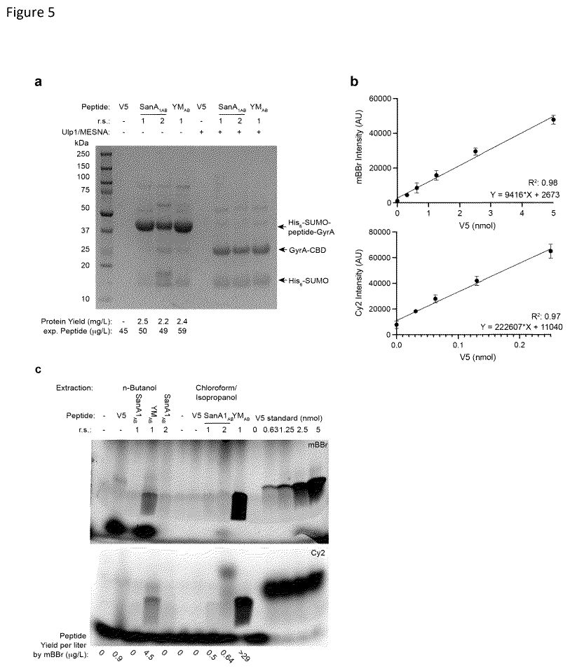

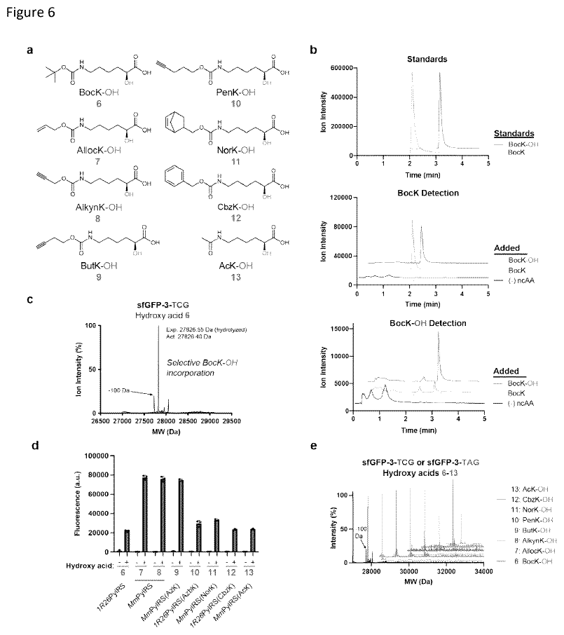

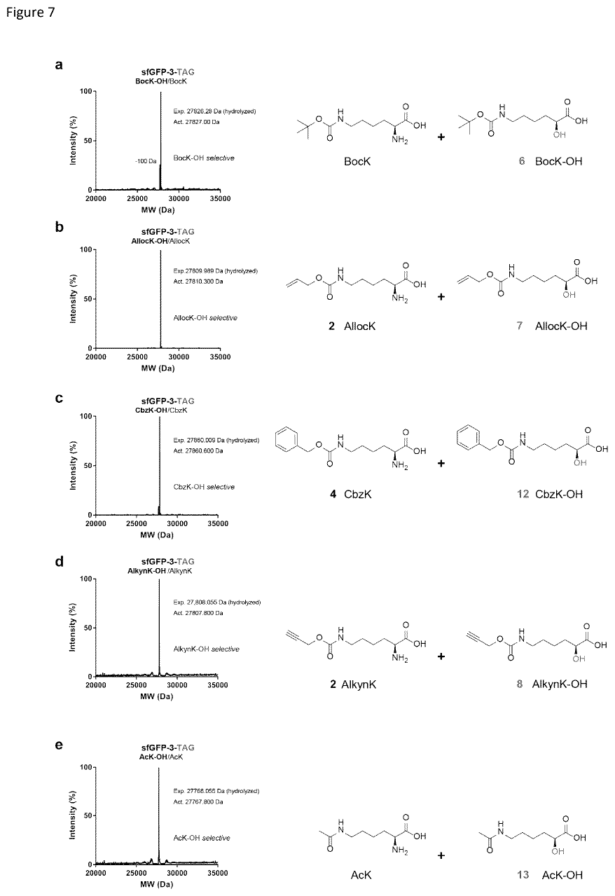

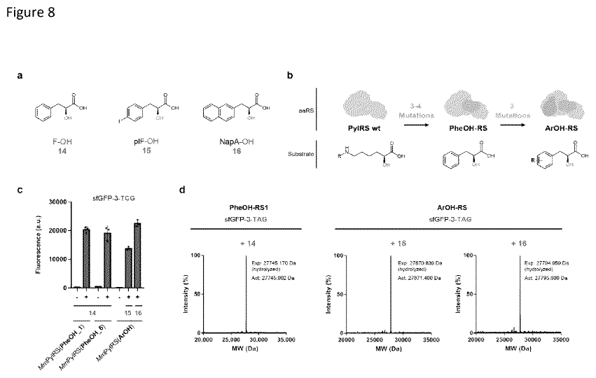

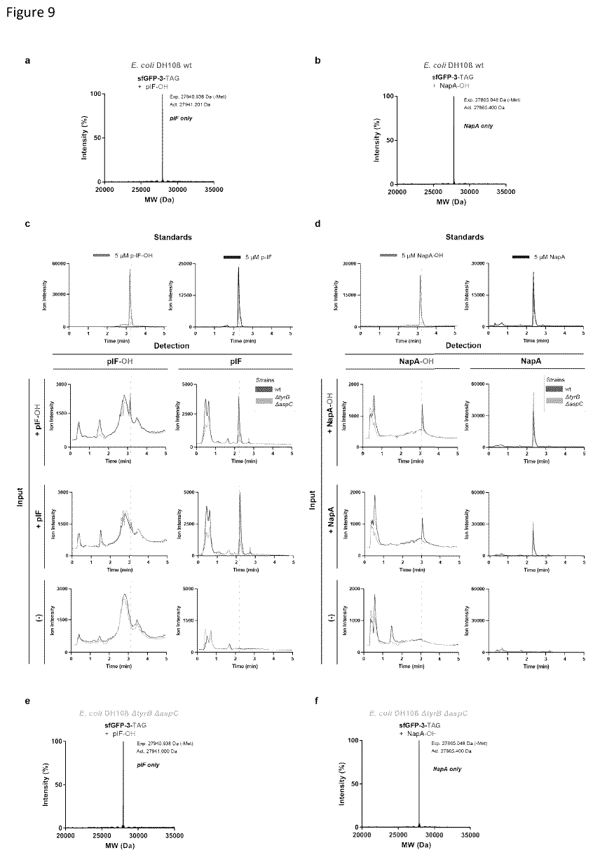

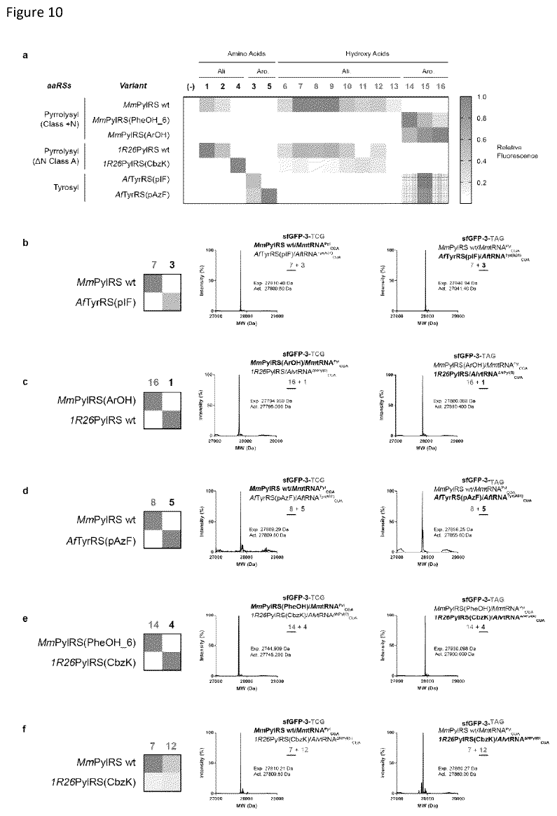

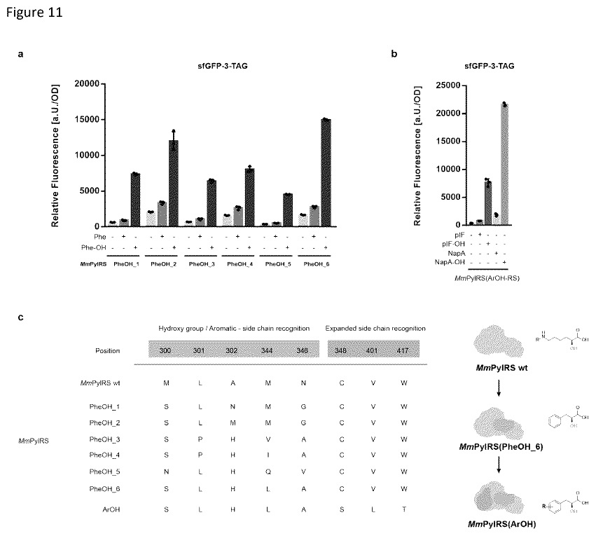

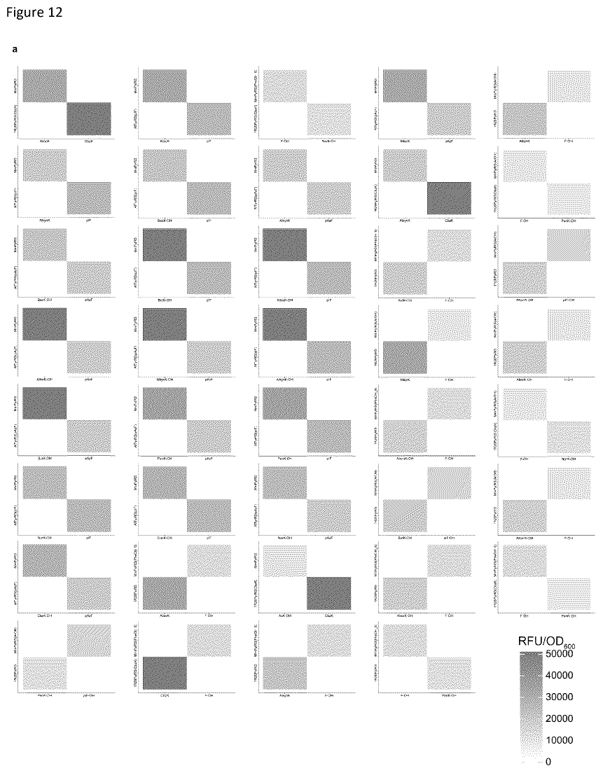

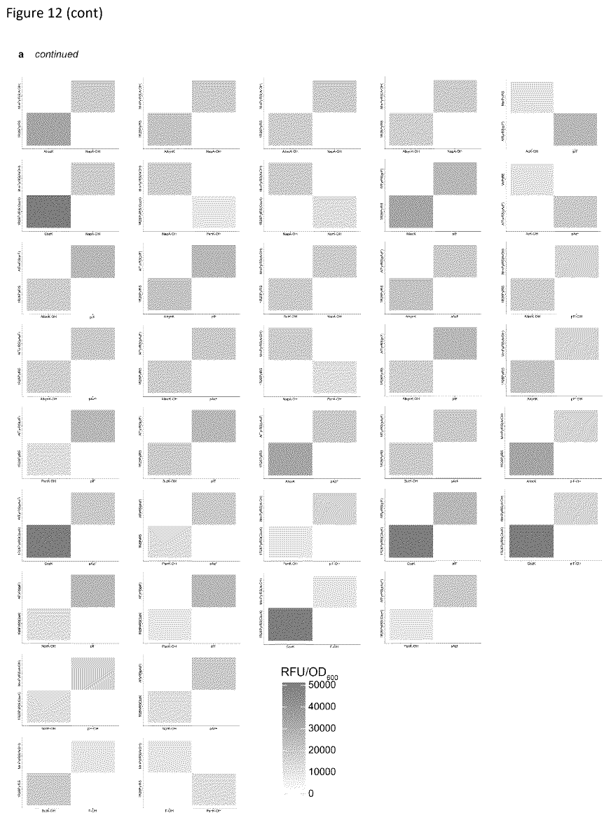

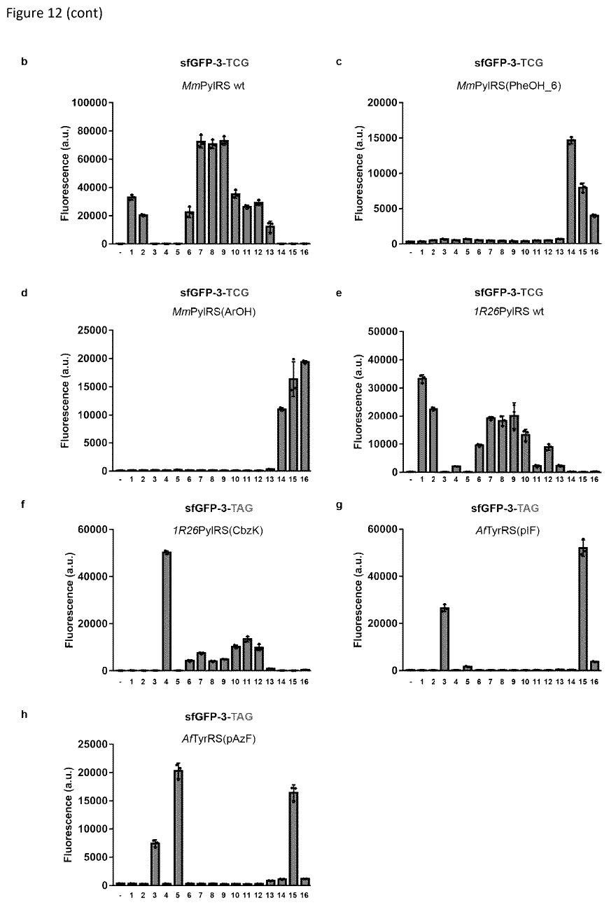

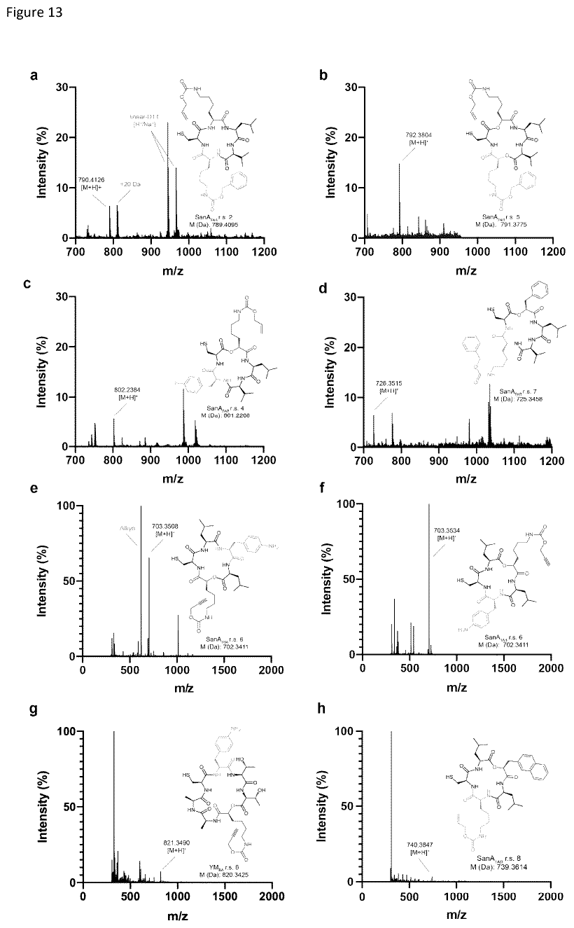

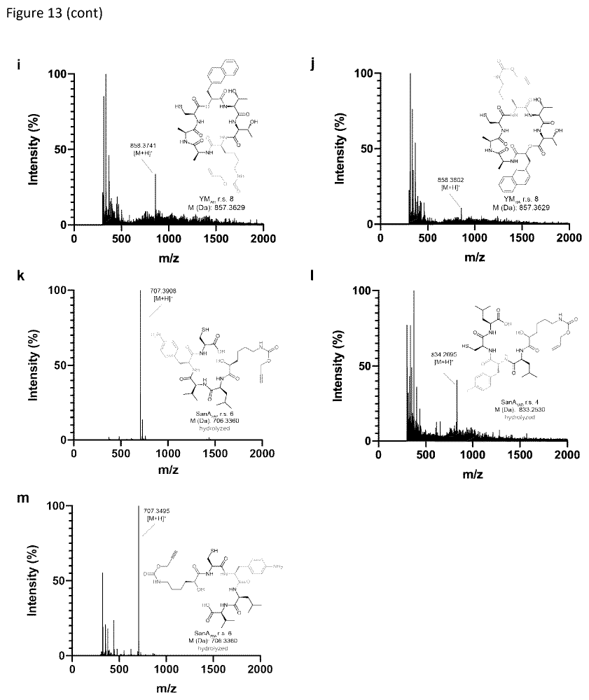

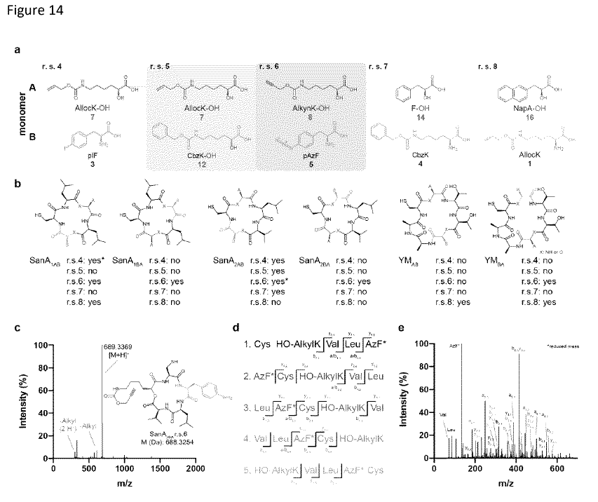

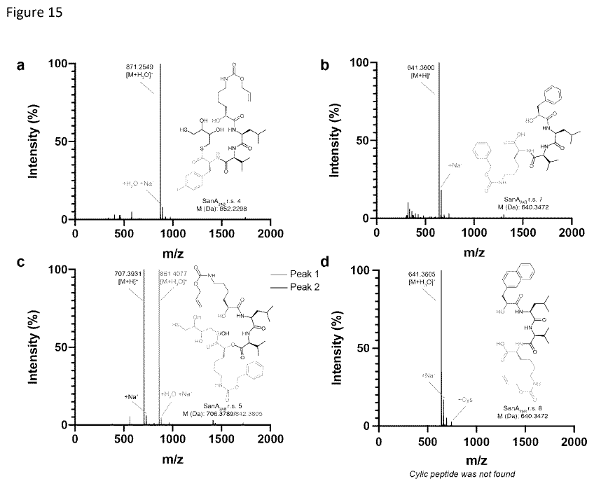

a, Yields of the Ni-NTA purified protein (mg/L) for all SanA1, SanA2 and YM sequences encoded in a His6-SUMO-peptide-GyrA-CBD fusion in combination with all reassignment schemes (r.s. 1-8, from FIG. 1, FIG. 5).

b, SDS-PAGE analysis of the Ni2+-NTA purified proteins for all reassignment schemes before and after treatment with Ulp1 protease and reductant (Ulp1/MESNA). Each lane contains 1% of the total purified protein or reaction mix. All samples were not heated to limit unspecific hydrolysis. The expected molecular weight for each of the bands before and after Ulp1/MESNA treatment is provided.

c, Yields of the Ni-NTA purified protein (mg/L) for the SanA3 and SanA4 sequences reported and corresponding SDS-PAGE gels of the purified protein before and after Ulp1/MESNA treatment.

d, SDS-PAGE analysis of the Ni2+-NTA purified proteins for the SanA3 and SanA4 sequences with reassignment schemes 1 and 3 before and after treatment with Ulp1/MESNA. Each lane contains 1% of the total purified protein or reaction mix. All samples were not heated to limit unspecific hydrolysis. The expected molecular weight for each of the bands before and after Ulp1/MESNA treatment is provided.

e, ESI-MS of three representative purified SUMO-SanA2AB-GyrA-CBD fusions using reassignment schemes 3-6. The expected (exp.) and observed (obs.) intact protein masses are shown.

FIG. 4. ESI-MS spectra of the isolated cyclic peptides encoding the non-canonical amino acids defined in r.s. 1-3.

a-c, Liquid chromatography mass spectrometry (LC-MS) data for all successfully isolated cyclic peptides produced using r.s. 1 (panel a), r.s. 2 (panel b) or r.s. 3 (panel c), as shown in FIG. 1, monomer A is coloured in blue and monomer B in green. The chemical structure and expected mass for each peptide is shown and coloured according to FIG. 1. The product peak for each peptide is labelled and other related peaks relative to the product peak. Traces of SUMO protein remaining from the reaction were labelled if detected. The spectra of SanA2 r.s. 2 is used as a reference in FIG. 13 and depicted there.

FIG. 5. Peptide production, extraction and quantification.

a, SDS-PAGE of purified His6-SUMO-peptide-GyrA-CBD fusion proteins and their cleavage products. Each protein was purified from a 500 mL expression culture and eluted in 1 mL (1% of this elution was loaded). The ‘protein yield’ in mg per L of culture was measured and the expected amount of peptide (exp. Peptide) was calculated based on the molecular weight of the protein and each cyclic peptide. A control with 22.5 μg of pure V5 peptide (V5) in 1 mL reaction buffer was prepared (1525 Da, theoretically 45 μg/L), 1% of this control was loaded alongside the purified protein. All samples were treated with Ulp1 and reductant (Ulp1/MESNA, +), and 1% of the reaction after 18 h incubation was loaded. This resulted in the near complete cleavage of His6-SUMO-peptide-GyrA-CBD to His6-SUMO and GyrACBD to release the peptide.

b, Fluorescence standard curve for monobromobimane (mBBr) and Alexa488-maleimide (Cy2 fluorescence) labelled V5 peptide; labelling reactions were performed in triplicate, run on SDSPAGE, and the V5 peptide labelling with the fluorophore quantified using the Fiji, Gel-tools software.

c, Fluorescence imaging of an SDS-PAGE gel separating the labelled extracts. Each lane corresponds to peptides (V5 control, SanA1AB r.s.1, SanA1AB r.s.2, YMAB r.s.1) extracted from 250 μL Ulp1/reductant reactions using n-Butanol or chloroform/isopropanol extraction (3:1). The extracts were labelled with monobromobimane (mBBr, +191 Da) or Alexa488-maleimide (Cy2, +718 Da). The amount of the extracted and labelled peptide was determined by quantifying the band intensity relative to the labelled V5 (mBBr) standards. An extracted yield in micrograms per L (μg/L) of the original protein expression culture was calculated to allow direct comparison with the expected yields in the cleavage reactions, and the known input concentration of the V5 peptide, both shown in panel a. The extraction yield of the V5 control peptide in n-butanol is 2%, and the V5 peptide is not extracted in chloroform/isopropanol. The extraction yield of YMAB r.s.1 (932 Da) in chloroform/isopropanol is 49%, while the extraction yield of YMAB r.s.1 in n-butanol is 8%. The extraction of SanA1AB r.s.1, SanA1AB r.s.2 is barely detectable even though the cleavage reactions (in panel a) indicate that similar amounts of these peptides and the YMAB r.s.1 peptide are present in the Ulp1 cleavage reactions. We note that this method labels free thiols and does not discriminate between cyclic and linear peptides. These experiments clearly demonstrate that extracted peptide yield is a function of the peptide sequence and the solvent used for extraction, and does not reflect the quantities of peptide in the Ulp1 cleavage reaction.

FIG. 6. Genetically encoding hydroxy acids in response to TAG and TCG codons in Syn61Δ3(ev5).

a, Structures of the ‘aliphatic’ hydroxy acids, 6-13, used in this study.

b, Intracellular detection of hydroxy acid 6 (BocK-OH) and its amino acid counterpart (BocK). Following incubation of E. coli DH10B cells with the relevant ‘Added’ compound (2 mM BocK-OH (red line) or 2 mM BocK (gray line)), extracts were subjected to LC-MS assays, with selected ion monitoring (SIM), to detect BocK (middle graph) or BocK-OH (bottom graph). In cells with BocK added (gray line), only BocK was detected. In cells with BocKOH added (red line), both BocK-OH and BocK were detected. Standards (100 μM) were used to define the retention times of BocK and BocK-OH (top graph). The minor peak at the 2 min retention time detected with the BocK-OH channel corresponds to the 13C isotope of BocK, which has the same mass as BocK-OH.

c, Production of sfGFP-His6 incorporating hydroxy acid 6 (BocK-OH), sfGFP(3BocK-OH)-His6, from a sfGFP3TCG gene in Syn61Δ3(ev5) cells harbouring the MmPylRS/MmtRNAPylCGA orthogonal pair was confirmed by electrospray ionization mass spectrometry (ESI-MS)—after purification under hydrolytic conditions, only a mass corresponding to hydroxy acid incorporation followed by ester bond cleavage at position 3 was detected. The expected mass of 27826.55 Da results from cleavage of the ester bond between residues 2 and 3 in the sfGFPHis6 protein. The smaller peak of −100 Da peak results from loss of tert-butoxycarbonyl from BocK-OH. The calculated mass of this protein prior to cleavage is 28,028.41 Da.

d, Orthogonal PyIRS active site variants direct the incorporation of hydroxy acids 6-13 into sfGFP. TCG or TAG codon readthrough in the absence (−) or presence of hydroxy acids was measured with a sfGFP-3-TCG or sfGFP-3-TAG reporter in Syn61Δ3(ev5) cells. The data shows the fluorescence from the resulting sfGFP expression. All MmPylRS variants paired with MmtRNAPylCGA to decode TCG codons, and all 1R26PyIRS variants paired with A/vRNAΔNPyl(8)CUA to decode TAG codons. MmPylRS(AzK) is MmPylRS-L309A-C348VY384F17, 1R26PyIRS(AzbK) is 1R26PyIRS-M129A-Y206F, MmPylRS(NorK) is MmPylRSY306G-Y384F-1405R22, 1R26PyIRS(CbzK) is 1R26PyIRS-Y126G-M129L21, MmPylRS(AcK) is MmPylRS-L301M-Y306L-L309A-C348F-L367M23.

e, Production of sfGFP-His6 incorporating hydroxy acids 6-13 confirmed by ESI-MS. Expressions were performed in Syn61Δ3(ev5) cells supplemented with a hydroxy acid and its cognate PyIRS/tRNA pair. Expected mass 6 (hydrolysed): 27826.26 Da, actual mass: 27826.40 Da. The smaller peak of −100 Da peak results from loss of tert-butoxycarbonyl from BocKOH. Expected mass 7 (hydrolysed): 27810.23 Da, actual mass: 27809.80 Da. Expected mass 8 (hydrolysed): 27808.20 Da, actual mass: 27809.60 Da. Expected mass 9 (hydrolysed): 27822.26 Da, actual mass: 27822.50 Da. Expected mass 10 (hydrolysed): 27836.29 Da, actual mass: 27836.00 Da. Expected mass 11 (hydrolysed): 27876.32 Da, actual mass: 27876.60 Da. Expected mass 12 (hydrolysed): 27860.28 Da, actual mass: 27860.20 Da. Expected mass 13 (hydrolysed): 27768.18 Da, actual mass: 27767.80 Da.

FIG. 7. Competition assay to measure the selectivity of pyrrolysyl tRNA synthetases for hydroxy acids over amino acids.

a-e, The M. mazei (Mm) and 1R26 orthogonal pyrrolysyl-tRNA synthetase (PyIRS)/tRNA pairs are selective for hydroxy acids over amino acids. In DH10B cells containing the MmPylRS/MmtRNAPylCUA pair, sfGFP-3-TAG was expressed in the presence of 2 mM (7.5 mM in the case of AlkynK, 10 mM in the case of AcK) amino acid and 2 mM (10 mM in the case of AcK-OH) of its corresponding hydroxy acid analogue. When supplemented with BocK and BocK-OH (panel a), AllocK and AllocK-OH (panel b), CbzK and CbzK-OH (panel c), AlkynK and AlkynK-OH (panel d), or AcK and AcK-OH (panel e) electrospray ionization mass spectrometry (ESI-MS) analysis of Ni2+-NTA purified sfGFP-3-TAG yielded a mass corresponding to the selective incorporation of the hydroxy acid over the amino acid. The −100 Da peak corresponds to loss of the tert-butoxycarbonyl in BocK. Analogously in cells containing the 1R26PyIRS(CbzK)/A/vRNAΔNPyl(8)CUA pair, ESI-MS analysis demonstrated the selectivity for CbzK-OH over CbzK (panel c).

FIG. 8. Directed evolution of PyIRS variants with specificity for hydroxy acids with aromatic side chains.

a, Structures of hydroxy acids with aromatic side chains attached to the beta carbon, 14-16, used in this study.

b, Strategy for generating PyIRS variants with specificity towards hydroxy acids with aromatic side chains. Directed evolution of MmPylRS (light grey) yielded variants MmPylRS(PheOH_1) and MmPylRS(PheOH_6), which selectively incorporate 14 (phenyllactic acid). Further directed evolution of MmPylRS(PheOH_6), using libraries that mutate amino acids in the enzyme involved in recognizing the side chain of the substrate, led to the discovery of ArOHRS. This variant enables the incorporation of hydroxy acids 15 and 16 that bear substituents on the phenyl ring. For sequences of evolved MmPylRS variants, see FIG. 9.

c, Activity of MmPylRS(PheOH_1), MmPylRS(PheOH_6) and MmPylRS(ArOH) with hydroxy acids bearing aromatic sidechains (14-16). The production of sfGFP was measured in Syn61Δ3(ev5) cells containing the indicated aaRS and cognate tRNA, an sfGFP-3-TCG gene and the indicated hydroxy acid.

d, sfGFP-His6 was produced in the presence of 14, 15, and 16, in DH10B cells transformed with sfGFP-3-TAG, the indicated aaRS and cognate tRNA. The identity of the monomer incorporated was verified via electrospray ionization mass spectrometry (ESI-MS). In all three cases, only the mass corresponding to hydroxy acid incorporation followed by hydrolysis of the ester bond at position 3 was detected, confirming the selectivity of each aaRS for the indicated hydroxy acid.

FIG. 9. Analysis of aromatic hydroxy acids intracellular concentrations and compatibility with orthogonal tyrosyl synthetase (TyrRS)/tRNATyr pairs.

a-b, TyrRS variants incorporate amino acid analogues in DH10B wt cells supplemented with hydroxy acids. Purified sfGFP-3-TAG samples expressed with the AfTyrRS(plF)/AtRNATyrCUA pair or the MjTyrRS(Nap)/MjtARNATyrCUA pair in the presence of the hydroxy acids 15 (plF-OH) or 16 (NapA-OH) were analysed by LC-MS, revealing the intact mass corresponding to the incorporation of the cognate amino acid. AfTyrRS(plF) is AfTyrRS-Y361-L69M-H74L-Q116E-D165T-1166G1, and MjTyrRS(Nap) is MjTyrRSY32L/D158P/1159A/L1162Q/A167V2.

c, Intracellular interconversion of 15 (plF-OH) determined by liquid chromatography mass spectrometry (LC-MS). Hydroxy and amino acids were detected by an LC-MS assay with selected ion monitoring (SIM) mode and compared to 5 μM standards of each compound. E. coli DH10B strains (wt in gray, ΔaspC/ΔtyrB metabolic knockout in light blue) were grown in the presence of 1 mM plF-OH (hydroxy acid, red traces) or 1 mM plF (amino acid, grey traces) for 18 h, from which cell lysates were extracted and injected into the LC-MS.

d, Intracellular interconversion of 16 (Nap-OH) determined by liquid chromatography mass spectrometry (LC-MS). Experiments were performed essentially as described in panel a.

e-f, TyrRS variants incorporate amino acid analogues in DH10B ΔaspC/ΔtyrB metabolic knockout cells supplemented with hydroxy acids. Purified sfGFP-3-TAG samples expressed with the AfTyrRS(plF)/AftRNATyrCUA pair or the MjTyrRS(Nap)/MjtARNATyrCUA pair in the presence of the hydroxy acids 15 (plF-OH) or 16 (NapA-OH) were analysed by LC-MS, revealing the intact mass corresponding to the incorporation of the corresponding amino acids.

FIG. 10. Defining the ncAA and hydroxy acid specificity of orthogonal aaRSs derived from mutually orthogonal pairs defines pairs of aaRS/tRNA pairs for incorporating distinct monomers in response to distinct codons in Syn61Δ3(ev5) cells.

a, Seven orthogonal aaRS variants and their cognate tRNAs, spanning three mutually orthogonal aaRS classes were tested for their ability to incorporate 16 monomers (5 ncAAs and 11 alpha hydroxy acids with aliphatic (ali.) and aromatic (aro.) sidechains) into sfGFP. The fluorescent protein was expressed from sfGFP-3-XXX (where XXX is TCG for Class +N pyrrolysyl-derived pairs, and TAG for ΔN Class A pyrrolysyl-derived pairs and tyrosyl-derived pairs) in Syn61Δ3(ev5) cells. Fluorescence levels within a row are normalized to the maximum activity of the active site variant assayed in that row; raw data is provided in FIG. 12b-h. Hashed squares indicate incorporations of amino acids in cells supplemented with the corresponding hydroxy acids. 1R26PyIRS(CbzK) is 1R26PyIRS-Y126G-M129L21, AfTyrRS(plF) is AfTyrRS-Y361-L69M-H74L-Q116E-D165T-1166G30, and AfTyrRS(pAzF) is AfTyrRS-Y36T-H74L-Q116E-D165T-1166G-N190K30.

b-f, Further characterizing five pairs of mutually orthogonal active sites derived from different synthetase types (Class+N pyrrolysyl, ΔN Class A pyrrolysyl, and tyrosyl), shown in a. In the presence of both aaRS/tRNA pairs and their cognate substrates, selective incorporation of each monomer (underlined) by each pair was confirmed by electrospray ionization mass spectrometry (ESI-MS) of sfGFP expressed from sfGFP-3-TCG or sfGFP-3-TAG reporters.

FIG. 11. Directed evolution of pyrrolysyl-tRNA synthetases that specifically incorporate hydroxy acids with aromatic side chains.

a, Specificity and activity of PheOH-RS variants. DH10B cells transformed with sfGFP-3-TAG and the respective PheOH-RS variant were grown in 2xYT media supplemented with 4 mM 14 phenyllactic acid (PheOH) or phenylalanine (Phe).

b, Specificity and activity of ArOH-RS. DH10B cells transformed with a sfGFP-3-TAG reporter and the respective PheOH-RS/MmtRNAPylCUA were grown in the presence of 2 mM 15 (plF-OH) and 16 (NapA-OH) or 2 mM of their respective amino acid analogues, showing strong specificity for the hydroxy analogues.

c, Sequences of PheOH-RS and ArOH-RS variants. Based upon crystal structures of MmPylRS, positions 300, 301, 302, 344, and 346 may be proximal to the hydroxy group and phenyl ring recognition; these positions were targeted during the first round of evolution—this yielded PheOH-RS1-6. As part of the next evolution stage, we targeted positions 348, 401, and 417.

FIG. 12. Identification of mutually orthogonal aaRS/tRNA pairs for encoding different combinations of two distinct non-canonical monomers.

a, We identified 77 mutually orthogonal aaRS/tRNA pairs which encode two distinct substrates, in which each pair recognizes a distinct substrate monomer. The absolute GFP florescence values (RFU/OD600) for indicated aaRS/tRNA monomer combinations are shown, combinations with both cognate activities >10000 RFU/OD600 and both non-cognate activities <1000 RFU/OD600 were considered a mutually orthogonal pair. From this analysis, we identified 77 pairs of mutually orthogonal aaRS/tRNA combinations which can encode 49 unique pairs of monomers; see also Table 1.

b-h, Raw data (RFU/OD600) bar graphs organized by aaRS active site variant. The raw data was used to generate the 2×2 orthogonality matrices in panel a and to generate the orthogonality matrix, following normalization, in FIG. 10.

FIG. 13. Annotated spectra of cyclic depsipeptides and their hydrolysis products.

a-d, The chemical structures and the annotated LC-MS spectra for the depsipeptides listed in FIG. 4b. Monomer A is coloured in blue and monomer B in green, the encoded ester bond is highlighted in red. Cyclic depsipeptides isolated as a fraction of hydrolysed non-cyclized sequence, the hydrolysed products are shown in FIG. 15. For the SanA2AB sequence, the depsipeptide spectrum (r.s. 5, red) was overlaid with its corresponding amino analogue (r.s. 2, black).

e-j, Annotated spectrum of all products observed as intact macrocyclic depsipeptide.

k-l, Depsipeptides observed only as hydrolysed linear products after hydrolytic cleavage of the encoded ester bond.

m, Detected traces of the hydrolysed cyclic peptide SanA2BA r.s. 6 from FIG. 14c.

FIG. 14. Genetically programmed cell-based synthesis of non-natural depsipeptide macrocycles.

a, Reassignment schemes 4-8 (r.s. 4-8) used for macrocyclic depsipeptide synthesis. The reassignment schemes define the identity of monomers A and B; monomer A (blue) is incorporated in response to the TCG codon and monomer B (green) is incorporated in response to the TAG codon.

b, Chemical structure of all cyclic depsipeptides, indicating the positions of non-canonical monomers A and B. For each reassignment scheme (r.s. 4-8), ‘yes’ indicates that we detected the exact mass of the depsipeptide following its purification, and ‘no’ indicates that we could not detect the peptide, an asterisk* indicates hydrolysis of the ester bond after successful cyclisation. The raw mass spectra as well as spectra are provided in FIG. 13. c, Mass spectrum and chemical structure of the depsipeptide SanA2BA r.s. 6; known fragment peaks are annotated relative to the [M+H]+ peak, trace hydrolysis was observed (FIG. 13m).

d-e, Expected linear sequences for the fragmentation of the cyclic peptide SanA2BA r.s. 6, all detected a-, b- and y-series fragments are annotated in the MS/MS spectra (panel e).

FIG. 15. Annotated mass spectra of isolated cyclic depsipeptides.

a-c, Structure and LC-MS spectra of the hydrolysed linear products observed as by-product in the cyclisation attempts of the depsipeptides depicted in FIG. 10. Hydrolysed products corresponding to the cyclic peptides in FIG. 14a-d.

d, Hydrolysed linear peptide of peptide SanA2AB r.s. 8; we did not observe the corresponding the cyclic peptide.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

The present invention requires at least two components: a suitably modified prokaryotic cells in which at least two codons can be reassigned to non-canonical, non-alpha-amino acids; and a heterologous nucleic acid which can encode a polymer having both peptide and non-peptide bonds.

### Prokaryotic Cell

As used herein, the term “prokaryotic cell”, in the context of the invention, refers to a prokaryotic cell which has been suitably modified in comparison to an unmodified, parental cell. Typically a prokaryotic cell will be produced by genetic modification of a pre-existing (i.e. “parental” or “parent”) cell. Thus, a prokaryotic cell may be derived from a parent cell, i.e. be identical to a parent cell, except for comprising one or more genetic modifications. The skilled person will be able to readily identify the parent cell on which a prokaryotic cell is based and the genetic modifications carried out. As used herein, a “parent cell” may be any naturally-occurring, commercially-available, deposited, catalogued or otherwise well-known cell, or derivative thereof.

A prokaryote is a unicellular organism that lacks a membrane-bound nucleus, mitochondria, or any other membrane-bound organelle. Prokaryotes are divided into two domains, Archaea and Bacteria. The genome of prokaryotic organisms generally is a circular, double-stranded piece of DNA, multiple copies of which may exist at any time.

Preferably, the prokaryotic cell of the present invention is a bacterial cell. Preferably the prokaryotic cell is suitable for heterologous protein production, in particular the production of polypeptides and non-peptide polymers comprising one or more canonical or non-canonical amino acids (for instance those described by Ferrer-Miralles, N. and Villaverde, A., 2013. Microbial Cell Factories, 12:113) and one or more non-alpha-amino acids. Suitable bacterial cells include: Escherichia (e.g. Escherichia coli), caulobacteria (e.g. Caulobacter crescentus), phototrophic bacteria (e.g. Rodhobacter sphaeroides), cold adapted bacteria (e.g. Pseudoalteromonas haloplanktis, Shewanella sp. strain Ac10), pseudomonads (e.g. Pseudomonas fluorescens, Pseudomonas putida, Pseudomonas aeruginosa), halophilic bacteria (e.g. Halomonas elongate, Chromohalobacter salexigens), streptomycetes (e.g. Streptomyces lividans, Streptomyces griseus), nocardia (e.g. Nocardia lactamdurans), mycobacteria (e.g. Mycobacterium smegmatis), coryneform bacteria (e.g. Corynebacterium glutamicum, Corynebacterium ammoniagenes, Brevibacterium lactofermentum), bacilli (e.g. Bacillus subtilis, Bacillus brevis, Bacillus megaterium, Bacillus licheniformis, Bacillus amyloliquefaciens), and lactic acid bacteria (e.g. Lactococcus lactis, Lactobacillus plantarum, Lactobacillus casei, Lactobacillus reuteri, Lactobacillus gasseri) cells. In some embodiments the prokaryotic cell is a gram-negative bacterial cell.

Preferably, the prokaryotic cell of the present invention is an Escherichia coli, Salmonella enterica, or Shigella dysenteriae cell. These are phylogenetically related species as disclosed by Lukjancenko, O., et al., 2010. Microbial ecology, 60(4), pp. 708-720; and Karberg, K. A., et al., 2011. PNAS, 108(50), pp. 20154-20159.

More preferably, the prokaryotic cell of the present invention is an E. coli cell. The parent cell may be any suitable E. coli, including K-12, MG1655, BL21, BL21(DE3), AD494, Origami, HMS174, BLR(DE3), HMS174(DE3), Tuner(DE3), Origami2(DE3), Rosetta2(DE3), Lemo21(DE3), NiCo21(DE3), T7 Express, SHuffle Express, C41(DE3), C43(DE3), and m15 pREP4 or derivatives thereof (Rosano, G. L. and Ceccarelli, E. A., 2014. Frontiers in microbiology, 5, p.172). Most preferably, the parent cell is MDS42, MG1655, or BL21 or a derivative thereof. MG1655 is considered as the wild type strain of E coli. The GenBank ID of genomic sequence of this strain is U00096. BL21 is widely available commercially. For example, it can be purchased from New England BioLabs with catalog number C2530H (https://www.neb.com/products/c2530-bl21-competent-e-coli).

Preferably one or more tRNA or release factors may be deleted from the prokaryotic cell and the cell may remain viable. For example, a tRNA which decodes only the one or more sense codons that have been replaced (or deleted) may be dispensable. Similarly, a tRNA which decodes the one or more sense codons that have been replaced (or deleted) may be dispensable if the remaining sense codons that it decodes may also be decoded by an alternative tRNA. For example, serT, encoding tRNA^{Ser}_{UGA}, is the only tRNA that decodes TCA codons in E. coli, and is therefore normally essential. However, if the genome of the prokaryotic cell does not contain TCA codons then serT may be dispensable.

Methods for modifying bacterial cells for the production of polymers comprising non-canonical amino acids are set forth in WO2020229592. Such methods are useful for producing prokaryotic cells useful for the production of polymers comprising non-alpha-amino acids.

When the genome of a cell has been modified to produce the synthetic prokaryotic genome of the present invention, the prokaryotic cell preferably does not display a substantially decreased growth rate. Thus, preferably the prokaryotic cell does not have a substantially decreased growth rate relative to the host cell comprising the parent genome. In some embodiments the prokaryotic cell has a doubling time less than 4 times, 3 times, 2 times, or about 1.6 times, slower than the parent cell. The doubling time can be determined by any method known to those of skill in the art. In some embodiments the doubling time is determined at 37° C., 25° C. or 42° C., in LB media.

When the genome of a cell has been modified to produce the prokaryotic cell of the present invention, the cell advantageously does not have any substantial phenotypical changes. Thus, preferably the prokaryotic cell does not have any substantial phenotypical changes relative to the host cell. In some embodiments the host cell comprising the synthetic prokaryotic genome has a mean cell length less than 100%, 50%, or about 20% greater than the host cell comprising the parent genome. For example, the cell length may be about 1.5 to 3 microns. The cell length can be determined by any method known to those of skill in the art.

### Codon Replacement

As used herein, a “sense codon” is a nucleotide triplet that codes for an amino acid. Thus, sense codons may be identified in the genome of a cell by gene prediction, i.e. by identifying regions of the genome that code for proteins (i.e. genes) and the corresponding open reading frames (ORFs). Typically, genomes naturally comprise 61 sense codons: GCT, GCC, GCA, GCG, CGT, CGC, CGA, CGG, AGA, AGG, AAT, AAC, GAT, GAC, TGT, TGC, CAA, CAG, GAA, GAG, GGT, GGC, GGA, GGG, CAT, CAC, ATT, ATC, ATA, TTA, TTG, CTT, CTC, CTA, CTG, AAA, AAG, ATG, TTT, TTC, CCT, CCC, CCA, CCG, TCT, TCC, TCA, TCG, AGT, AGC, ACT, ACC, ACA, ACG, TGG, TAT, TAC, GTT, GTC, GTA, and GTG (read from 5′ to 3′ on the coding strand of DNA). The standard genetic code encodes the 20 canonical amino acids using the 61 triplet codons. 18 of the 20 amino acids are encoded by more than one synonymous codon (see FIG. 17). The one or more sense codons may be one or more native sense codons, i.e. sense codons which are present in the parent genome.

The 61 sense codons in DNA are transcribed into corresponding mRNA and subsequently decoded by one or more tRNAs. tRNAs carry an amino acid to a ribosome as directed by the sense codons in the mRNA. The tRNAs can recognise one or more sense codons via a complementary anticodon. A sequence of sense codons is subsequently translated into a polypeptide (i.e. a sequence of amino acids).

Preferably, the genome-wide removal of the one or more sense codons, but not other sense codons, enables all the cognate tRNA corresponding to said one or more sense codons to be deleted without removing the ability to decode the one or more sense codons remaining in the genome. Thus, the one or more sense codons may be selected from: TCG, TCA, AGT, AGC, GCG, GCA, GTG, GTA, CTG, CTA, TTG, TTA, ACG, ACA, CCG, CCA, CGG, CGA, CGT, CGC, AGG, AGA, GGG, GGA, GGT, GGC, ATT, and ATC.

Aminoacyl-tRNA synthetases for serine, leucine and alanine do not recognize the anticodons of their cognate tRNAs. This may facilitate the assignment of codons within these boxes to new amino acids through the introduction of tRNAs bearing cognate anticodons that do not direct mis-aminoacylation by endogenous synthetases. Thus, the one or more sense codons may be selected from: TCG, TCA, TCT, TCC, AGT, AGC, GCG, GCA, GCT, GCC, CTG, CTA, CTT, CTC, TTG, and TTA.

Preferably, the one or more sense codons fulfill both these criteria, thus the one or more sense codons may be selected from: TCG, TCA, AGT, AGC, GCG, GCA, CTG, CTA, TTG, and TTA. More preferably, the one of more sense codons are selected from TCG, TCA, AGT, AGC, TTG, TTA, GCG and GCA. Most preferably, the one of more sense codons are TCG and/or TCA.

Preferably, one or more sense codons are removed such that the genome of the prokaryotic cell is compatible with codon reassignment to non-canonical amino acids and/or non-alpha-amino acids. Thus, the one or more sense codons may comprise one or more of TCA, CTA, or TTA. Alternatively, two or more sense codons are removed, wherein the two or more sense codons comprise one or more of the sense codon pairs, selected from the group consisting of: GCG and GCA; GCT and GCC; TCG and TCA; AGT and AGC; TCT and TCC; CTG and CTA; TTG and TTA; and CTT and CTC. Preferably, two or more sense codons are removed, wherein the two or more sense codons comprise one or more of the sense codon pairs, selected from the group consisting of: GCG and GCA; TCG and TCA; AGT and AGC; CTG and CTA; and TTG and TTA. More preferably, the two or more sense codons comprise TCG and TCA.

To achieve removal of sense codons they may be replaced with synonymous sense codons. This is preferable to ensure that the encoded protein sequence of the cell genome is not changed. The person skilled in the art is able to deduce suitable synonymous sense codon replacements. For example, in E. coli, typically TCG, TCA, TCT, TCC, AGT and AGC all encode serine; typically GCG, GCA, GCT and GCC all encode alanine; typically CTG, CTA, CTT, CTC, TTG and TTA all encode leucine.

In some embodiments, the replacement is a defined replacement, i.e. one sense codon is replaced with a single synonymous sense codon.

For example, the defined replacement may be: GCG replaced with either GCT or GCC; GCA replaced with either GCT or GCC; TCG replaced with any one of TCT, TCC, AGT, or AGC; TCA replaced with any one of TCT, TCC, AGT, or AGC; AGT replaced with any one of TCG, TCA, TCT, or TCC; AGC replaced with any one of TCG, TCA, TCT, or TCC; CTG replaced with any one of CTT, CTC, TTG or TTA; CTA replaced with any one of CTT, CTC, TTG or TTA; TTG replaced with any one of CTG, CTA, CTT or CTC; or TTA replaced with any one of CTG, CTA, CTT or CTC. Preferably the one or more defined sense codon replacements are selected from one or more of: GCG to either GCT or GCC; GCA to either GCT or GCC; TCG to either AGT or AGC; TCA to either AGT or AGC; AGT to either TCA or TCT; AGC to either TCG or TCC or TCA; TTG to CTT; and TTA to CTC. More preferably, TCG and/or TCA are replaced with AGC and/or AGT. Most preferably, TCG is replaced with AGC and/or TCA is replaced with AGT.

Preferably, the defined replacement is such that the genome is compatible with codon reassignment to non-alpha-amino acids. For example: (i) GCG may be replaced with either GCT or GCC, and GCA may be replaced with either GCT or GCC; (ii) TCG may be replaced with any of TCT, TCC, AGT, or AGC, and TCA may be replaced with any of TCT, TCC, AGT, or AGC; (iii) AGT may be replaced with any of TCG, TCA, TCT, or TCC, and AGC may be replaced with any of TCG, TCA, TCT, or TCC; (iv) CTG may be replaced with any of CTT, CTC, TTG or TTA, and CTA may be replaced with any of CTT, CTC, TTG or TTA; or (v) TTG may be replaced with any of CTG, CTA, CTT or CTC, and TTA may be replaced with any of CTG, CTA, CTT or CTC.

Preferably, the defined replacement scheme is one or more of those listed in the table below:

| | |
|---|---|
| Codon 1 | Codon 2 |

| From | To | From | To |
|---|---|---|---|
| GCG | GCT | GCA | GCT |
| GCG | GCT | GCA | GCC |
| GCG | GCC | GCA | GCT |
| GCG | GCC | GCA | GCC |
| TCG | TCT | TCA | TCT |
| TCG | TCT | TCA | TCC |
| TCG | TCT | TCA | AGT |
| TCG | TCT | TCA | AGC |
| TCG | TCC | TCA | TCT |
| TCG | TCC | TCA | TCC |
| TCG | TCC | TCA | AGT |
| TCG | TCC | TCA | AGC |
| TCG | AGT | TCA | TCT |
| TCG | AGT | TCA | TCC |
| TCG | AGT | TCA | AGT |
| TCG | AGT | TCA | AGC |
| TCG | AGC | TCA | TCT |
| TCG | AGC | TCA | TCC |
| TCG | AGC | TCA | AGT |
| TCG | AGC | TCA | AGC |
| AGT | TCG | AGC | TCG |
| AGT | TCG | AGC | TCA |
| AGT | TCG | AGC | TCT |
| AGT | TCG | AGC | TCC |
| AGT | TCA | AGC | TCG |
| AGT | TCA | AGC | TCA |
| AGT | TCA | AGC | TCT |
| AGT | TCA | AGC | TCC |
| AGT | TCT | AGC | TCG |
| AGT | TCT | AGC | TCA |
| AGT | TCT | AGC | TCT |
| AGT | TCT | AGC | TCC |
| AGT | TCC | AGC | TCG |
| AGT | TCC | AGC | TCA |
| AGT | TCC | AGC | TCT |
| AGT | TCC | AGC | TCC |
| CTG | CTT | CTA | CTT |
| CTG | CTT | CTA | CTC |
| CTG | CTT | CTA | TTG |
| CTG | CTT | CTA | TTA |
| CTG | CTC | CTA | CTT |
| CTG | CTC | CTA | CTC |
| CTG | CTC | CTA | TTG |
| CTG | CTC | CTA | TTA |
| CTG | TTG | CTA | CTT |
| CTG | TTG | CTA | CTC |
| CTG | TTG | CTA | TTG |
| CTG | TTG | CTA | TTA |
| CTG | TTA | CTA | CTT |
| CTG | TTA | CTA | CTC |
| CTG | TTA | CTA | TTG |
| CTG | TTA | CTA | TTA |
| TTG | CTG | TTA | CTG |
| TTG | CTG | TTA | CTA |
| TTG | CTG | TTA | CTT |
| TTG | CTG | TTA | CTC |
| TTG | CTA | TTA | CTG |
| TTG | CTA | TTA | CTA |
| TTG | CTA | TTA | CTT |
| TTG | CTA | TTA | CTC |
| TTG | CTT | TTA | CTG |
| TTG | CTT | TTA | CTA |
| TTG | CTT | TTA | CTT |
| TTG | CTT | TTA | CTC |
| TTG | CTC | TTA | CTG |
| TTG | CTC | TTA | CTA |
| TTG | CTC | TTA | CTT |
| TTG | CTC | TTA | CTC |
| GCT | GCG | GCC | GCG |
| GCT | GCG | GCC | GCA |
| GCT | GCA | GCC | GCG |
| GCT | GCA | GCC | GCA |
| TCA | TCG | | |
| TCA | TCT | | |
| TCA | TCC | | |
| TCA | AGT | | |
| TCA | AGC | | |
| TCT | TCG | TCC | TCG |
| TCT | TCG | TCC | TCA |
| TCT | TCG | TCC | AGT |
| TCT | TCG | TCC | AGC |
| TCT | TCA | TCC | TCG |
| TCT | TCA | TCC | TCA |
| TCT | TCA | TCC | AGT |
| TCT | TCA | TCC | AGC |
| TCT | AGT | TCC | TCG |
| TCT | AGT | TCC | TCA |
| TCT | AGT | TCC | AGT |
| TCT | AGT | TCC | AGC |
| TCT | AGC | TCC | TCG |
| TCT | AGC | TCC | TCA |
| TCT | AGC | TCC | AGT |
| TCT | AGC | TCC | AGC |
| CTA | CTG | | |
| CTA | CTT | | |
| CTA | CTC | | |
| CTA | TTG | | |
| CTA | TTA | | |
| TTA | CTG | | |
| TTA | CTA | | |
| TTA | CTT | | |
| TTA | CTC | | |
| TTA | TTG | | |
| CTT | CTG | CTC | CTG |
| CTT | CTG | CTC | CTA |
| CTT | CTG | CTC | TTG |
| CTT | CTG | CTC | TTA |
| CTT | CTA | CTC | CTG |
| CTT | CTA | CTC | CTA |
| CTT | CTA | CTC | TTG |
| CTT | CTA | CTC | TTA |
| CTT | TTG | CTC | CTG |
| CTT | TTG | CTC | CTA |
| CTT | TTG | CTC | TTG |
| CTT | TTG | CTC | TTA |
| CTT | TTA | CTC | CTG |
| CTT | TTA | CTC | CTA |
| CTT | TTA | CTC | TTG |
| CTT | TTA | CTC | TTA |

Preferably, none of these codon replacements affect ribosomal binding sites (AGGAGG), which are highly conserved regulatory sequences in E. coli. The selected codon replacements may be tested on a small test region (e.g. a 20 kb region of the genome rich in both essential target genes and target codons) to assess viability. If the codon replacements are not viable on the small test region they may be disregarded.

When replacement of one or more sense codons in the parent cell genome with defined replacement synonymous sense codons does not result in a viable genome, alternative replacement synonymous sense codons may be used. For instance, 99.9% of the occurrences of one or more sense codons in the parent cell genome may be replaced with a defined (i.e. single) synonymous sense codon, and the remaining 0.1% with alternative synonymous sense codons. For example, 99.9% of the occurrences of TCG may be replaced with AGC and 0.1% replaced with TCT, TCC, AGT or AGC; and/or 99.9% of the occurrences of TCA may be replaced with AGT and 0.1% replaced with TCT, TCC, AGT or AGC.

Preferably 90% or more, 95% or more, 98% or more, 99% or more, 99.5% or more, 99.6% or more, 99.7% or more, 99.8% or more, 99.9% or more, or 100% of the occurrences of the one or more sense codons in the unmodified prokaryotic cell are replaced with synonymous sense codons. In some embodiments 90% or more, 95% or more, 98% or more, 99% or more, 99.5% or more, 99.6% or more, 99.7% or more, 99.8% or more, 99.9% or more, or 100% of the occurrences of TCG and/or TCA in the unmodified prokaryotic cell are replaced with AGC and/or AGT, most preferably 90% or more, 95% or more, 98% or more, 99% or more, 99.5% or more, 99.6% or more, 99.7% or more, 99.8% or more, 99.9% or more, or 100% of the occurrences of TCG in the unmodified parent prokaryotic cell are replaced with AGC and/or 90%, 95%, 90% or more, 95% or more, 98% or more, 99% or more, 99.5% or more, 99.6% or more, 99.7% or more, 99.8% or more, 99.9% or more, or 100% of the occurrences of TCA in the unmodified parent prokaryotic cell are replaced with AGT.

As used herein, a “stop codon” or “nonsense codon” is a nucleotide triplet that codes for termination of translation into proteins. Typically, genomes naturally comprise 3 stop codons: TAA (“ochre”), TGA (“opal” or “umber”) and TAG (“amber”).

In some embodiments the prokaryotic cell genome further comprises 10 or fewer, 5 or fewer, or no occurrences of one or two stop codons, preferably 10 or fewer, 5 or fewer, or no occurrences of the amber stop codon (TAG). Preferably wherein 90% or more, 95% or more, 98% or more, 99% or more, or all of the occurrences of TAG in the parent prokaryotic genome are replaced with TAA (the ochre stop codon). In preferred embodiments the synthetic prokaryotic genome comprises no occurrences of the amber stop codon (TAG), optionally wherein all of the occurrences of TAG in the parent prokaryotic genome are replaced with TAA (the ochre stop codon).

Accordingly, in preferred embodiments the prokaryotic cell genome of the present invention comprises no occurrences of one or more, or two or more sense codons and no occurrences of one stop codon, preferably the amber stop codon (TAG). In more preferred embodiments the prokaryotic cell genome of the present invention comprises no occurrences of two sense codons, preferably TCG and TCA, and no occurrences of the amber stop codon (TAG), optionally wherein TCG, TCA and TAG in the parent prokaryotic genome are replaced with synonymous codons, for example 99.9% or more of the occurrences of TCG in the parent prokaryotic cell genome are replaced with AGC, 99.9% or more of the occurrences of TCA in the parent prokaryotic cell genome are replaced with AGT and all of the occurrences of TAG in the parent prokaryotic cell genome are replaced with TAA.

### Codon Reassignment and Orthogonal tRNA Synthetases

In some embodiments the one or more sense codons (i.e. those removed from the parent genome) are reassigned to encode alternative canonical amino acids. For example, if TCG and TCA have been removed, one or both may be reassigned to encode a monomer other than serine, in the case of the invention a non-alpha-amino acid.

For instance, the genome of the prokaryotic cell of the present invention substantially or completely lacks one or more sense codons. Therefore, one or more tRNA or release factors may be deleted from the synthetic genome. For instance, a tRNA which decodes the one or more sense codons that have been replaced (or deleted) may be deleted from the synthetic prokaryotic genome. A tRNA which decodes one or more sense codons that have been replaced (or deleted) may be deleted and the prokaryotic cell will remain viable if the tRNA decodes only the one or more sense codons that have been replaced (or deleted); or alternatively if the tRNA decodes one or more sense codons that have been replaced (or deleted) and one or more sense codons that have not been replaced (or deleted), if the tRNA is dispensable for the one or more sense codons that have not been replaced (or deleted) (i.e. the one or remaining sense codons which the tRNA decodes are decoded by one or more alternative tRNAs). For example, if the prokaryotic cell genome lacks TCA sense codons, serT, encoding tRNA^{Ser}_{UGA}, may be deleted and/or if the prokaryotic cell genome lacks TCG sense codons, serU, encoding tRNA^{Ser}_{UGA}, may be deleted. The deletion of one or more tRNAs may be used, for instance, in combination with an orthogonal aminoacyl-tRNA synthetase/tRNA pair to reassign the one or more sense codons to a non-alpha-amino acid.

For example, if TCG and TCA have been removed from the synthetic prokaryotic genome, serT, encoding tRNA^{Ser}_{UGA}, and serU, encoding tRNA^{Ser}_{UGA}, may be deleted from the synthetic prokaryotic genome, and either the tRNA_{CGA} can be reassigned (e.g. to tRNA^{Ala}_{CGA}) an orthogonal aminoacyl-tRNA synthetase/tRNA_{CGA} pair may be introduced to the host cell (e.g. by a heterologous nucleic acid or by incorporation into the synthetic prokaryotic genome) to reassign TCG to a non-alpha-amino acid. Thus, the prokaryotic cell of the present invention further comprises one or more reassigned tRNAs and/or one or more heterologous nucleotides (e.g. plasmids) encoding one orthogonal aminoacyl-tRNA synthetase (aaRS)-tRNA pair. In some embodiments the host cell of the present invention further comprises a plasmid encoding an orthogonal aminoacyl-tRNA synthetase (aaRS)-tRNA pair. Alternatively, the orthogonal aminoacyl-tRNA synthetase (aaRS)-tRNA pair may be introduced into the host cell by incorporation into the genome of the prokaryotic cell. Thus, in some embodiments the prokaryotic cell genome encodes an orthogonal aminoacyl-tRNA synthetase (aaRS)-tRNA pair, preferably wherein the gene encoding the native tRNA has been deleted from the prokaryotic cell genome. In preferred embodiments the prokaryotic cell of the present invention further comprises one or more reassigned tRNAs. Methods for reassigning tRNAs will be well known to those of skill in the art.

Thus, the present invention provides for use of a prokaryotic cell according to the present invention for producing polymers comprising one or more non-alpha-amino acids, preferably two or more non-alpha-amino acids, most preferably three or more non-alpha-amino acids.

As used herein, “noncanonical amino acids” are amino acids that are not naturally encoded or found in the genetic code. Despite the use of only 22 amino acids by the translational machinery to assemble proteins (the proteinogenic amino acids—20 in the standard genetic code and an additional 2 that can be incorporated by special translation mechanisms), over 140 amino acids are known to occur naturally in proteins and thousands more may occur in nature or be synthesized in the laboratory. Thus, non-canonical amino acids may comprise any amino acid excluding L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan and L-tyrosine. Unnatural amino acids additionally exclude L-pyrrolysine and L-selenocysteine.

In some embodiments, the non-canonical amino acids are unnatural amino acids (UAAs).

Suitable non-canonical amino acid and UAAs will be well known to those of skill in the art, for example those disclosed in Neumann, H., 2012. FEBS letters, 586(15), pp. 2057-2064; and Liu, C. C. and Schultz, P. G., 2010. Annual review of biochemistry, 79, pp. 413-444. In some embodiments the non-proteinogenic amino acid and/or UAAs are selected from one or more of: p-Acetylphenylalanine, m-Acetylphenylalanine, O-allyltyrosine, Phenylselenocysteine, p-Propargyloxyphenylalanine, p-Azidophenylalanine, p-Boronophenylalanine, O-methyltyrosine, p-Aminophenylalanine, p-Cyanophenylalanine, m-Cyanophenylalanine, p-Fluorophenylalanine, p-lodophenylalanine, p-Bromophenylalanine, p-Nitrophenylalanine, L-DOPA, 3-Aminotyrosine, 3-lodotyrosine, p-lsopropylphenylalanine, 3-(2-Naphthyl)alanine, Biphenylalanine, Homoglutamine, D-tyrosine, p-Hydroxyphenyllactic acid, 2-Aminocaprylic acid, Bipyridylalanine, HQ-alanine, p-Benzoylphenylalanine, o-Nitrobenzylcysteine, o-Nitrobenzylserine, 4,5-Dimethoxy-2-nitrobenzylserine, o-Nitrobenzyllysine, o-Nitrobenzyltyrosine, 2-Nitrophenylalanine, Dansylalanine, p-Carboxymethylphenylalanine, 3-Nitrotyrosine, Sulfotyrosine, Acetyllysine, Methylhistidine, 2-Aminononanoic acid, 2-Aminodecanoic acid, Pyrrolysine, Cbz-lysine, Boc-lysine and Allyloxycarbonyllysine.

Non-alpha-amino acids are monomers that lack the RCH(NH₂)COOH structure of an amino acid, and in particular lack the alpha (NH₂) group. Alpha hydroxy acids are exemplary non-alpha-amino acids. Some alpha hydroxy acids are hydroxy variants of canonical amino acids. Alternatively, they may be non-canonical acids. Exemplary alpha-hydroxy acids include hydroxy acids with aromatic side-chain, optionally selected from F-OH, plF-OH and NapA-OH; and alpha-hydroxy acids with an aliphatic side-chain, optionally selected from BocK-OH, PenK-OH, AllocK-OH, NorK-OH, AlkynK-OH, CbzK-OH, ButK-OH and AcK-OH. Hydroxy-acid analogues of O4BBy, O2beY, pCaaF, pVsaf, pAaF are also contemplated. See (lannuzzelli and Fasan, Chem. Sci., 2020, 11,6202).

Genetic code expansion uses an orthogonal aminoacyl-tRNA synthetase (aaRS)-tRNA pair to direct the incorporation of non-proteinogenic amino acids into proteins, in response to an unassigned codon (e.g. the amber stop codon, UAG) introduced at the desired site in a gene of interest. The orthogonal synthetase does not recognize endogenous tRNAs, and specifically aminoacylates an orthogonal cognate tRNA (which is not an efficient substrate for endogenous synthetases) with the monomer provided to (or synthesized by) the cell (Chin, J. W., 2017. Nature, 550(7674), 53-60). The person skilled in the art would be able to identify and/or generate suitable orthogonal aminoacyl-tRNA synthetase (aaRS)-tRNA pairs (e.g. Elliott, T. S. et al., 2014. Nat Biotechnol 32, 465-472; Elliott, T. S., et al., 2016. Cell Chem Biol 23, 805-815; and Krogager, T. P. et al., 2018. Nat Biotechnol 36, 156-159). Thus, in some embodiments, the prokaryotic cell of the present invention further comprises one or more heterologous nucleotides (e.g. plasmids) encoding one orthogonal aminoacyl-tRNA synthetase (aaRS)-tRNA pair. In preferred embodiments the prokaryotic cell of the present invention further comprises a plasmid encoding an orthogonal aminoacyl-tRNA synthetase (aaRS)-tRNA pair. Alternatively, the orthogonal aminoacyl-tRNA synthetase (aaRS)-tRNA pair may be introduced into the prokaryotic cell by incorporation into the prokaryotic cell genome. Thus, in some embodiments the prokaryotic genome encodes an orthogonal aminoacyl-tRNA synthetase (aaRS)-tRNA pair, preferably wherein the gene encoding the native tRNA has been deleted from the parent prokaryotic genome.

Thus, in some embodiments the prokaryotic cell of the present invention further comprises one or more heterologous nucleotides (e.g. plasmids) which comprise one or more genes comprising said sense codons. In preferred embodiments the host cell further comprises a plasmid comprising a gene comprising said sense codons. The one or more sense codons may be present in a desired site in the gene, preferably wherein the desired site results in incorporation of one or more non-alpha-amino amino acids into polymers, which may also comprise canonical and non-canonical amino acids.

In other embodiments said sense codons may be present in one or more genes in the prokaryotic cell genome (for example, the heterologous nucleotide may be incorporated into the prokaryotic cell genome). The one or more sense codons may be present in a desired site in the gene, preferably wherein the desired site directs incorporation of one or more non-alpha-amino acids into polymers.

For example, if TCG and TCA have been removed from the synthetic prokaryotic genome, serT, encoding tRNA^{Ser}_{UGA}, and serU, encoding tRNA^{Ser}_{UGA}, may be deleted from the prokaryotic cell genome, and an orthogonal tRNA synthetase/tRNA_{CGA} pair may be used in combination with (heterologous) genes comprising the TCG codon, to encode polypeptides comprising one or more non-alpha-amino acid. Thus, the host cell of the present invention may, for instance, further comprise: (i) a plasmid encoding an orthogonal aminoacyl-tRNA synthetase/tRNA_{CGA} pair; and (ii) a plasmid comprising a gene comprising one or more TCG codons. Similarly, if AGT and AGC are removed, serV, encoding tRNA^{Ser}_{UGA} may be deleted from the synthetic prokaryotic genome, and an orthogonal aminoacyl-tRNA synthetase/tRNA_{ACU} pair and/or an orthogonal aminoacyl-tRNA synthetase/tRNA_{GCU} pair may be used. Similarly, if CTG and CTA are removed, leuP,Q,T,V encoding tRNA^{Leu}_{CAG}, and leuW, encoding tRNA^{Leu}_{CAG}, may be deleted from the prokaryotic cell genome, and an orthogonal aminoacyl-tRNA synthetase/tRNA_{CAG} pair may be used. Similarly, if TTG and TTA are removed, leuX, encoding tRNA^{Leu}_{CAG}, and leuZ, encoding tRNA^{Leu}_{CAG}, may be deleted from the prokaryotic cell genome, and an orthogonal aminoacyl-tRNA synthetase/tRNA_{CAA} pair and/or an orthogonal aminoacyl-tRNA synthetase/tRNA_{UAA} pair may be used may be used. Similarly, if GCG and GCA are removed, alaT,U,V, encoding tRNA^{Ala}_{UGC} may be deleted from the prokaryotic cell genome, and an orthogonal aminoacyl-tRNA synthetase/tRNA_{CGC} pair may be used.

In some embodiments the prokaryotic cell genome lacks genes encoding release factors (e.g. RF1) and/or the host cell lacks release factors (e.g. RF1) to increase the efficiency of incorporation of non-proteinogenic amino acids.

In order to incorporate two non-alpha-amino acids, it is necessary to use two different tRNA synthetase/tRNA pairs, which must be mutually orthogonal so as to avoid cross-incorporation of different monomers. Thus, a first orthogonal tRNA synthetase—tRNA pair and a second orthogonal tRNA synthetase—tRNA pair may be introduced into a prokaryotic cell of the invention. The first tRNA may decode one of the sense codons removed from the genome, and the second tRNA may decode another sense codon removed from the genome, and/or a nonsense codon removed from the genome. The orthogonal tRNA synthetases specifically charge their orthogonal cognate tRNA with a non-alpha-amino acid, which may be an alpha-hydroxy acid. Hence, the prokaryotic cell may contain a system wherein two or more sense codons have been repurposed to code for non-alpha-amino acids.

Orthogonal tRNA synthetase enzymes and paired tRNAs may be obtained from any suitable source. Organisms from which aaRS may be derived include Methanosarcina mazei, Archeoglobus fulgidus, Methanomethylophilus sp, Methanocaldococcus jannaschii and Methanosarcina barkeri. For example, orthogonal aaRS/tRNA pairs include Methanosarcina mazei (Mm)PylRS/MmtRNA^{Pyl}_{CGA}, Archeoglobus fulgidus (Af)TyrRS(plF)/AftRNA^{Tyr(A01)}_{CUA}, Methanomethylophilus sp. 1 R26 (1 R26)PyIRS(CbzK)/AlvtRNA^{ΔNPyl(8)}_{CUA} and Methanocaldococcus jannaschii (Mj)TyrRS(Nap)/MjtRNA_{TyrCUA}. Methanosarcina barkeri PyIRS has also been used to incorporate hydroxy acids.

Preferably, the aaRS mutants used in the present invention do not incorporate significant amounts of amino acids, for example do not incorporate phenylalanine.

Preferably, a suitable aaRS is selected for its ability to interact with alpha-hydroxy acids; for example, its specificity for the amino group of amino acids may be less stringent. The aaRS may then be mutagenized to improve its specificity for a desired alpha-hydroxy acid.

Advantageously, the aaRS is mutagenized in order to select variants which are able to incorporate different monomers. For example, a library of aaRS can be constructed by randomising positions M300, L301, A302, M344 and N346 in MmPylRS with degenerate codons. Further mutagenesis can be used to select mutants better able to incorporate hydroxy acids having aromatic side chains; preferably, mutations are selected for at positions C348, V401 and W417, which delimit the pocket to the enzyme which binds to the substrate sidechain. Other mutations can be introduced, based on the principle of variation of the enzyme in the region which interacts with the sidechain of the monomer.

### Reassignment Schemes

Once a codon has been selected for reassignment, the aaRS and cognate tRNA can be selected in order to determine which monomer is incorporated at the defined codon position. Thus, although the nucleic acid encoding the polymer will determine the sequence of the monomers in the polymer, the aaRS and cognate tRNA which incorporate the monomer will determine the identity of the incorporated monomer.

It has been shown that certain aaRS are more selective for certain monomers, and in addition certain aaRS/tRNA pairs are mutually orthogonal in their acylation activity. Thus reassignment schemes can be devised which promote incorporation of different monomers at desired locations in the polymer without cross-reacting to incorporate undesired monomers.

Advantageously, therefore, the first and second reassigned codons are recognised by orthogonal tRNAs, which in turn are acylated by aaRS enzymes which are mutually orthogonal in their monomer specificity.

### Alpha-Hydroxy Acids

In general, alpha-hydroxy acids can be derived from canonical or noncanonical amino acids. For example, alpha-hydroxy acids include, but are not limited to, p-hydroxy-L-phenyllactic acid (the alpha-hydroxy analogue of tyrosine), leucic acid (the alpha-hydroxy analogue of leucine), lactic acid (the alpha-hydroxy analogue of alanine), 2-hydroxy-3-methylbutyric acid (the alpha-hydroxy analogue of valine), 2-hydroxy-3-phenylpropionic acid, the hydroxy derivative of phenylalanine (F-OH), and alpha-hydroxy analogues of other natural and unnatural amino acids. Derivatives of noncanonical amino acids include the hydroxy derivatives of NE-Alloc-L-lysine (AllocK-OH), p-lodo-phenylalanine (plF-OH), Nε-((Prop-2-yn-1-yloxy)carbonyl)-L-lysine (AlkynK-OH), p-Azido-Phenylalanine (pAzF-OH), L-3-(2-Naphthyl)alanine (NapA-OH), Ne-tert-butoxycarbonyl-L-lysine (BocK-OH), N6-Carbobenzyloxy-L-lysine (CbzK-OH) and other lysine derivatives including PenK-OH, NorK-OH, ButK-OH and AcK-OH.

### Depsipeptides

A depsipeptide is a polypeptide in which one or more peptide (C(O)NHR) bonds have been replaced with an ester (C(O)OR) bond. Depsipeptides are found in nature, and are typically produced by nonribosomal synthesis by nonribosomal peptide synthetases (NRPSs) or by post-translational modification of canonical amino acids. Many naturally occurring depsipeptides are cyclic, such as anticancer agent romidepsin, antibiotic agent etamycin, and anti-HIV agents papuamides A and B.

Other depsipeptides include Sansalvamide A, N-methylsansalvamide and neo-N-methylsansalvamide, Alternaramide, Zygosporamide, Enniatins and Beauvericin, Beauvenniatins, Hirsutellide A, Kutznerides, Monamycins, Himastatin, Paecilodepsipeptide A and Conoideocrellide A, Pullularins A-E, Hirsutatins A and B, HUN-7293, Bassianolide, Verticlide and Emodepside, Emodepside, BZR-cotoxins I-IV, Aureobasidins and Clavariopsin A and B. See Sivanathan and Scherkenbeck, Molecules (2014) 19, 12368-12420.

### Cyclic Polymers

The invention provides for the production of cyclic polymers, including macrocycles and cyclic depsipeptides. Methods of cyclisation of peptides are known in the art, and can be employed in the present invention. In embodiments, cyclic polymers may be cyclised post-translationally in a separate step, by at least partly isolating the linear polymer form the prokaryotic cell and inducing cyclisation. In other embodiments, cyclisation may take place in the cell such that a cyclic polymer is produced without further intervention.

In one embodiment, the polymer is expressed in fusion with an intein or parts thereof, which can excise itself and join the remaining sections of the polymer to form a cyclic polymer. For example, polymers of the present invention can be expressed in fusion with SUMO and GyrA reporters, as described below.

Systems for cyclisation based on inteins are further described in Tavassoli, A. & Benkovic, S. J. Nature Protocols 2, 1126-1133, doi:10.1038/nprot.2007.152 (2007).

Systems may also be based on subtiligase, as described in Weeks, A. & Wells, J., Chem. Rev. 2020,120,3127-3160.

### Nucleic Acids

The invention provides tools and methods (the claimed prokaryotic cells and methods described herein) which are intended for the production of polymers which can comprise non-alpha-amino acids, as well as both canonical and non-canonical amino acids. The use of codon reassignment allows the selection of the location of non-canonical monomers in the encoded sequence, by modification of the translation machinery of the cell as opposed to modification of the coding sequence of a nucleic acid encoding the desired polymer.

Thus, prokaryotic cells according to the invention advantageously express nucleic acids which encode a polymer comprising at least two non-alpha-amino acids. Codon reassignment in the host prokaryotic cell can determine the position and nature of the non-alpha-amino acid monomers in the encoded polymer.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

### EXAMPLES

### Methods

### Gene Recoding

For experiments in Syn61Δ3 derived cells, for all genes and plasmids it was necessary to compress the genetic code according to the recoding rules of Syn61¹⁸. For all protein CDSs, we recoded TCG to AGC, TCA to AGT, and TAG to TAA using a custom Python script¹⁸. For all plasmids used in this study, please see Table 2 for sequence details.

### Construction of aaRS/tRNA Plasmids for Decoding TCG or TAG Codons in Syn61Δ3

To incorporate a single ncAA, we used a pMB1-based plasmid encoding the orthogonal aaRS/tRNA pair that directs ncAA incorporation in response to TCG or TAG codons. For MmtRNAPylCGA constructs which incorporate aliphatic monomers in response to the TCG codon, we designed pylT tRNA genes which included pylTopt mutations³⁸ and which replaced the entire anticodon stem loop (ASL—the anticodon plus 6 nucleotides on either side) with the ASL sequence of the serU (in the case of CGA) E. coli gene3. We constructed pMB1-based aaRS/tRNA plasmids by HiFi Assembly of multiple fragments, including i) a recoded aaRS under control of the constitutive glnS promoter, synthesized as a gBlock (IDT), and ii) a recoded pMB1 plasmid backbone generated via PCR from previous recoded aaRS/tRNA plasmids3; this backbone included a recoded kanR gene, a pMB1 vector origin, and an orthogonal tRNA gene with an Ipp promoter and an rrnC terminator. With this assembly approach we generated all novel aaRS/tRNA plasmids from this study; see Table 2 for sequence details. For double distinct ncAA incorporations, we designed pMB1-based plasmids which polycistronically encoded two aaRS/tRNA pairs^{52, 16}. Briefly, we added two aaRS CDSs downstream of a g/nS promoter, for which we optimized the intergenic region between the aaRS genes with an RBS calculator program (https://salislab.net/software/). For the polycistronic tRNA operon, we placed both tRNA genes downstream of an Ipp promoter; the intergenic region between tRNA genes was based upon the intergenic region between the alaX and alaW genes in the E. coli genome.

### Construction of Vectors for Expressing His6-SUMO-ncPeptide-GyrA

All cloning was conducted in E. coli DH10B. We generated plasmids for expressing SUMOpeptide-GyrA reporters based upon a recoded pBAD expression system. ncPeptide sequences were introduced by QuikChange® PCR or by the ligation of phosphorylated primers with a PCR-amplified and Aral-digested SUMO-GyrA plasmid backbone. The final plasmid contained i) a recoded SUMO-ncPeptide-GyrA gene, and ii) a recoded pBAD plasmid backbone containing apraR, araC, and a p15a vector origin region. sfGFP reporter plasmids were generated as described previously⁵². See Table 2 for sequence details.

### Generating DaspCΔtyrB Cells

We used a two-step lambda-red recombineering approach for the scarless deletion of the aspC and tyrB genes in E. coli DH10B cells. Briefly, the first recombination introduced the pheS*-HygR double selection cassette to delete the target gene, and the second recombination introduced a repair template to replace the double selection cassette with the native genomic environment of the targeted locus. We PCR amplified a pheS*-HygR double selection cassette with primers containing 50 bp flanking homology arms to the genomic landing site of interest. We then integrated the pheS*-HygR cassette at the designated genomic loci in DH10B cells harbouring the pKW20_CDFtet_pAraRedCas9_tracrRNA plasmid expressing the lambda-red alpha/beta/gamma genes. To initiate recombination, we prepared electrocompetent cells and electroporated 3 μg of the purified PCR product into 100 μL of DH10B cells pre-induced to express the lambda-red recombination machinery, as described previously⁵. We induced the arabinose promoter-controlled recombination machinery with L-arabinose added at 0.5% for 1 hour starting at OD600=0.2; pre-induced cells were then electroporated and recovered for 1 hour at 37° C. in 4 mL of super optimal broth (SOB) medium. Cells were then diluted into 50 mL of LB medium with 10 μg/mL tetracycline and grown for 4 hours at 37° C., 200 rpm. The cells were subsequently spun down, resuspended in 4 mL of H₂O, serially diluted, plated and incubated overnight at 37° C. on LB agar plates containing 10 μg/mL tetracycline and 200 μg/mL hygromycin. We constructed the aspC and tyrB repair templates through overlap extension PCR; this was introduced during the second recombination with the same method as described above, except that the LB agar plates contained 2.5 mM p-Cl-Phe in place of hygromycin. We verified the scarless deletions of aspC and tyrB by genotyping with primers flanking the locus of interest; sequential deletion of aspC and tyrB through this two-step lambda-red process yielded the DH10B ΔaspC/ΔtyrB cells. Primers used to generate DH10B ΔaspC/ΔtyrB cells are provided in Table 2.

### Measuring the Intracellular Levels of Amino Acids or Hydroxy Acids

We measured the intracellular concentration of non-canonical amino and hydroxy acids with an LC-MS method previously described⁵³. Briefly, we supplemented amino or hydroxy acids (1 mM final concentration) to a 5 mL solution of LB inoculated with diluted DH10B cells; a negative control sample was prepared in parallel without amino or hydroxy acid addition. We grew the samples at 37° C. while shaking (220 rpm) for 12 h. After growth, we measured the OD600 of each culture, and we then harvested the cells from each culture. We washed, centrifuged, and resuspended the cell pellets for three cycles with 1 mL of ice-cold LB media. For each washed cell pellet, we then resuspended them in a methanol:water solution (60:40) and added Zirconium beads (0.1 mm) to each suspension. We then lysed the suspensions by vortexing for 12 min, and we obtained clarified lysate following centrifugation at 21000×g for 30 min at 4° C. We then pipetted the clarified lysate supernatant carefully into a fresh 1.5 mL microcentrifuge tube, and we subjected the sample to another round of centrifugation at 21000×g for 2 h at 4° C. For LC-MS analysis, we transferred a 100 μL aliquot of the supernatant into 250 μL glass inserts (Agilent) and injected 5 μL from the resulting sample into an Agilent 1260 Infinity LC equipped with a Zorbax C18 (4.6×150 mm) column. After running the column run with a gradient of 0.5% to 95% acetonitrile in water, the sample was then injected into an Agilent 6130 Quadrupole LC-MS unit with the mass spectrometer set to selected ion monitoring (SIM) mode in order to detect the m/z value for the relevant amino or hydroxy acid.

### sfGFP Expression Measurements

We expressed sfGFP genes bearing single TCG or TAG codons at position 3 in Syn61Δ3(ev5) cells harbouring plasmids encoding aaRS/tRNA genes, as described previously3. Briefly, we co-electroporated 50 μL of Syn61Δ3(ev5) cells (all expressions utilized Syn61Δ3(ev5) cells) with a pMB1-based aaRS/tRNA plasmid (100 ng) and recovered them for 1.5 h in 1 mL of SOB while shaking at 1000 rpm at 37° C. Subsequently, we inoculated the cells (1 mL) into 6 mL of 2xYT media supplemented with 50 μg/mL kanamycin and incubated the cells overnight at 37° C. while shaking at 220 rpm. Following recovery, we then prepared the cells as electrocompetent for the subsequent electroporation of a pBAD_sfGFP reporter plasmid (100 ng) and recovered the cells in deep well 96-well plates in the presence of 50 μg/mL kanamycin and 50 μg/mL apramycin. Similarly, cells were prepared as chemically competent prior to transformation (described below). After 36 h of recovery, we setup expressions in 96-well microtiter plate format, inoculating overnight cultures 1:100 into 0.5 mL of 2xYT containing kanamycin (50 μg/mL), apramycin (50 μg/mL), L-arabinose (0.2%), and the presence or absence of ncAA. All ncAAs were supplemented to a final concentration of 2 mM, except AlkynK (2) amino acid was supplemented at 7.5 mM, AcK-OH (13) which was supplemented at 10 mM, and F-OH (15) which was supplemented at 4 mM. We incubated the 96-well plates for 16 hours at 37° C. while shaking at 750 rpm in a Thermo-Shaker (Grant-bio). We centrifuged the plates for 10 min at 3200 g and subsequently resuspended the cell pellets with 150 μL of PBS. To measure sfGFP expression normalized by cell density, we transferred 100 μL of resuspended cells to a Costar clear 96-well flat-bottom plate, and recorded OD600 and GFP fluorescence (λex: 485 nm; λem: 520 nm) measurements on a PHERAstar FS plate reader (BMG LABTECH) (gain setting of 0, focal adjustment of 00 mm).

### Purification of His6x-Tagged sfGFP Proteins from E. coli

E. coli cells (Syn61Δ3(ev5), DH10B, or DH10B ΔaspC/ΔtyrB) harbouring a pMB1-based aaRS/tRNA plasmid and a pBAD_sfGFP p15a-based plasmid were grown for 16 h while shaking (220 rpm) at 37° C. in the same media conditions described as for the sfGFP expression measurements (see above). To prepare overnight cultures, we used 25 mL volumes for sfGFP-3-TCG or sfGFP-3-TAG. After expression, we centrifuged cultures in lysis buffer (1×PBS, 1×Bugbuster® Protein Extraction Reagent (Novagen®) with a volume of 1/20th of the original culture supplemented with 50 μg/mL DNase 1, 20 mM imidazole, and 100 μg/mL lysozyme. We incubated the cell resuspensions at 4° C. for 30 min and then clarified the lysates by centrifugation for 30 min at 4° C. and 16000×g. We subsequently transferred the clarified lysate into 1.5 mL microcentrifuge tubes containing 50 μL of Ni2+-NTA slurry (Qiagen) and incubated the mixture for 1 h while tumbling at 4° C. After incubation, we collected the Ni2+-NTA beads by gravity filtration on a fritted column and resuspended them three times in 500 μL of wash buffer (PBS, 40 mM imidazole, pH 8). For polyhistidine-tagged protein elution, we added 100 μL of elution buffer (PBS, 300 mM imidazole, pH 8) to the beads and centrifuged the mixture (1000×g, 4° C., 1 min) in order to collect the eluate through the fritted column into a fresh 1.5 mL microcentrifuge tube. We repeated this elution three times and stored purified sfGFP protein at −20° C. prior to downstream analyses.

### Preparation of Chemically Competent Syn61Δ3(Ev5) Cells and Heat Shock Transformation

100 mL of LB medium were inoculated with 3 mL of Syn61Δ3(ev5) overnight culture and grown while agitated (200 rpm, 37° C.) to an OD600 of 0.3-0.4 (˜3 h). The cells were chilled on ice for 10 min and harvested by centrifugation (4000 rpm, 5 min, 4° C.), and the pellet was resuspended in 0.1 M CaCl2 and incubated on ice for at least 30 min. The cells were collected as before, resuspended in a total volume of 4 mL 0.1 M CaCl2 supplemented with 10% Glycerol and incubated on ice for another 5 min. The cells were aliquoted in 100 μL portions on ice and flash frozen until further use. For heat shock transformation the frozen cells were thawed on ice and 40 μL of cells were mixed with about 100 ng of plasmid DNA. The cells were heat shocked in a water bath (42° C.) for 45 s and then chilled for 5 min on ice. 500 μL of SOC medium was added and the cells were recovered for 1.5 h at 37° C. while shaking at 1000 rpm. The culture was then diluted into 3 mL of selective LB medium with the relevant antibiotic and grown for 36 h while shaking (200 rpm) at 37° C.

### Site-Saturation Mutagenesis of aaRS Libraries

Degenerate codons for site saturation mutagenesis were introduced into the MmPylRS gene on a pMB1 plasmid backbone via enzymatic inverse PCR (eiPCR)7, using primers containing a Bsal restriction site (see Supplemental Table 2 for primer details). Following restriction digest and re-ligation, libraries were transformed into electrocompetent DH10B cells ensuring a transformation of efficiency of at least 109 clones. Quality control to ensure library diversity was performed through Sanger sequencing of individual clones.

### aaRS Selections and Screening

Libraries were transformed into freshly prepared electrocompetent DH10B cells containing a CAT-D111-TAG—sfGFP-N150-TAG p15A dual selection plasmid ensuring a minimal transformation efficiency of 109. Overnight cultures were diluted in 2xYT media containing the corresponding hydroxy acid. After growing to OD600=1.0, cells were plated on varying concentrations of chloramphenicol (ranging from 100 to 400 μg/ml) in the presence of the corresponding hydroxy acid. Surviving colonies were picked and diluted into media containing 0.2% L-arabinose in the presence and absence of hydroxy acid, and sfGFP production was monitored as previously stated. Samples showing expression dependent on the presence of hydroxy acid were pooled, and plasmid DNA was purified. After selectively digesting the CAT-D111-TAG—sfGFP-N150-TAG p15A dual selection plasmid, the aaRS library was retransformed into DH10B cells harbouring sfGFP-3-TAG and plated on 0.2% L-arabinose in the presence of hydroxy acid. Greenly fluorescent clones were picked, and the individual plasmid DNA was miniprepped (Qiagen). Selectivity and activity for the corresponding hydroxy acid was monitored for each cloned by retransformation with sfGFP-3-TAG and monitoring sfGFP production as described above.

### Expression of the Target Peptide as His6-SUMO-Peptide-GyrA-CBD Fusion Protein

Chemically competent cells of Syn61Δ3(ev5) were transformed in two steps: i) The pMB1-based aaRS/tRNA plasmid (Kanamycin, 50 μg/mL) was transformed and new chemically competent cells were prepared for each aaRS/tRNA used; ii) each aaRS/tRNA was then combined with each pBAD-based expression plasmid (Apramycin, 50 μg/mL) used in the respective experiment. Following the overnight recovery of 1.5 mL of cells harbouring both the aaRS/tRNA and expression plasmids, 50 mL of selective TB medium was added and the cells were grown to OD600=2.5-3.0 (12 h) at 37° C. while being agitated (200 rpm). Expression was induced by addition of 0.4% L-arabinose and 2 mM each of the non-canonical amino acid or hydroxy acid (8 mM for F-OH (15)). The expression was harvested by centrifugation (10 min, 3200 g) after another 12 h. The pellets were washed with PBS and stored at −20° C. See Table 2 for plasmid details.

### His6-SUMO-Peptide-GyrA-CBD Purification, Cyclization and Extraction of Macrocyclic (Depsi-)Peptides and LC-MS Analysis

The cell pellets were thawed and resuspended in 30 mL MOPS lysis buffer (20 mM MOPS pH 6.9, 150 mM NaCl, 0.01 mg/mL DNAse and 0.1 mg/mL Lysozyme). The resuspended pellet was lysed by sonication in an ice bath (30×5 s pulses every 5 s, 50% Amplitude) and the cell lysate was cleared from debris by centrifugation (25000 rpm, 25 min, 4° C.). To the supernatant 100 μL Ni2+-NTA slurry (50% beads in 20% ethanol) was added and the suspension was incubated for 1 h at 4° C. under gentle agitation. The Ni2+-NTA beads were collected by gravity filtration through a fritted Poly-Prep column (BioRad) and washed with 20 mL MOPS wash buffer (20 mM MOPS pH 6.9, 300 mM NaCl, 40 mM imidazole). The protein was then eluted in 100 μL MOPS buffer (20 mM MOPS pH 6.9, 150 mM NaCl) supplemented with 200 mM imidazole. The protein concentration was determined after exchanging the buffer for 20 mM MOPS pH 6.9, 150 mM NaCl by measuring absorption at 280 nm and corrected for the predicted extinction coefficient (ProtParam, Expasy). Before and after the cyclisation reaction 2% of each sample was mixed with NuPAGE™ LDS Sample Buffer (20 μL total, final concentration 1×) and a total of 1% elution (10 μL) was applied per lane onto a 4-12% Bis-Tris SDS-Gel (NuPAGE) and run at 220 V for 28 min. The gel was stained with InstanBlue (Expedeon) and imaged after destaining with water using standard setting for Coomassie blue on a ChemiDoc (BioRad).

### Sample Preparation for LC-MS Analyses of Ulp1 Reaction Products

For cyclisation, all purified His6-SUMO-Peptide-GyrA-CBD from a single reaction was diluted to 500 μL with MOPS buffer (20 mM MOPS pH 6.9, 150 mM NaCl) and the cyclisation reaction initiated by addition of 0.005 mg/mL Ulp1 and 100 mM 1,4-dithiothreitol (DTT). The reaction was incubated at 37° C. for 18 h. The resulting cyclic peptides or depsipeptides were extracted through addition of 10% acetic acid (10 μL) and 200 μL of 3:1 chloroform/isopropanol per 500 μL reaction. The sample was mixed thoroughly and the emulsion mixture separated by centrifugation (1 min, 20000 rpm). The bottom organic phase was carefully transferred to a fresh 2 mL Eppendorf tube, the solvent was then evaporated under a strong airflow (within 1 h). The dried film was stored at −20° C. or directly resuspended in 20 μL 0.2% acetic acid to prepare for LC-MS analyses.

### Fluorescence Labelling and Quantification of Peptide Reaction Products (YMAB r.s. 1, SanA1AB r.s. 1 and r.s. 2)

The peptides YMAB r.s. 1, SanA1AB r.s. 1 and r.s. 2 were expressed on the 500 mL scale and purified as described as above. The elution buffer was exchanged to 20 mM MOPS pH 6.9, 150 mM NaCl, and the protein concentration was determined by measuring absorption at 280 nm and corrected for the predicted extinction coefficient (ProtParam, Expasy). A control reaction was prepared from commercial V5 peptide (Sigma), where 22.5 μg was diluted in 1 mL reaction buffer (20 mM MOPS pH 6.9, 150 mM NaCl). Ulp1/2-Mercaptoethanesulfonic acid sodium salt (MESNA) excision reaction was initiated by addition of 50 μL MESNA (200 mM stock) and 5 μL Ulp1 (1 mg/mL stock) to all samples and the samples were incubated for 18 h at 37° C. 20 μL (2%) of the total reaction was collected before and after the incubation with Ulp1/MESNA and mixed with NuPAGE™ LDS Sample Buffer (30 μL total, final concentration 1×). A total of 1% of the SDS-sample (15 μL) was applied per lane onto a 4-12% Bis-Tris SDS-Gel (NuPAGE) and a current of 220 V was applied for 28 min. The gel was stained with InstanBlue (Expedeon) and imaged after destaining with water using standard settings for Coomassie blue on a ChemiDoc imager (BioRad). The Ulp1/MESNA reaction was split into two 500 μL samples which were extracted separately with n-butanol8 or chloroform/isopropanol (3:1). Each sample was acidified with 25 μL 10% acetic acid and extracted 3 times with 200 μL of either organic solvent. The organic phase of each extraction was collected and washed once with 5 M NaCl. The water phase was discarded and the organic phase evaporated under vacuum using a Concentrator Plus (Eppendorf, n-butanol: 45° C. and setting for water 3-4 h, chloroform/isopropanol: RT and highly volatile setting, 15-30 min). The dried film was resuspended in 20 μL labelling buffer (50 mM Tris pH 8, 5 mM 35 EDTA) by vortexing followed by incubation at 37° C. for 30 min while vigorously shaking (1000 rpm). The sample was collected by centrifugation (5 min, 20000 rpm) and evenly divided into two amber Eppendorf tubes. To each tube, 1 μL of a cysteine reactive fluorophore was added (100 mM monobromobimane, MedChemExpress, or 10 mM Alexa Fluor™ 488 C5 Maleimide, Cy2 similar, Invitrogen™); the labelling reaction was left for one hour at RT. The Cy2 reaction was quenched for 1 h at RT by addition of 10 mM MESNA, mBBr does not require quenching and was kept at RT for another hour. 5 μL NuPAGE™ LDS Sample Buffer (16 μL total, final concentration 1.3×) was added to each sample and the full samples was separated on a Novex™ 16% Tricine SDS-Gel (Invitrogen™) in parallel with the extracted peptide. A standard curve of labelled V5 peptide was prepared (0-5 nmol, 1:2 dilution) and loaded alongside the sample. A voltage of 120 V was applied for 85 min, the gels were briefly rinsed with water and immediately imaged after the run completed. Cy2 fluorescence was imaged using the standard settings for Cy2 on a Typhoon™ imager (Amersham), while mBBr was imaged using the standard settings for ethidium bromide on a ChemiDoc imager (Bio-Rad). Note that the gels were not stored submerged in water and instead kept wet in a small amount of water. The gel bands were analysed using Fiji (Image J, version 2.1.0/1.53c) built-in gel tools.

### Peptide Quantification by Gel Fluorescence Intensity

A standard curve for both fluorophores was prepared from labelled V5 peptide. A 1 mM V5 stock solution (Sigma, 1 nmol per μL) was prepared in 20 mM Tris, 10 mM EDTA, and used as internal standard in all experiments. The standard curves for monobromobimane (mBBr, MedChemExpress) and Alexa Fluor™ 488 C5 Maleimide (Cy2, Invitrogen™) were prepared in triplicates and ranged from 0-5 nmol or 0-0.25 nmol, respectively. A 30 μL reaction was prepared in amber reaction tubes and contained 0-15 (mBBr) or 0-0.75 nmol V5 peptide (Cy2) and 10 mM mBBr (100 mM in acetonitrile) or 1 mM Cy2 (10 mM in DMSO) in 1×labelling buffer (50 mM Tris pH 8, 5 mM EDTA, 10× stock). The reaction was run for 1 h after which the Cy2 reaction was quenched by addition of 10 mM MESNA (200 mM stock), both reactions were incubated for another hour. NuPAGE™ LDS Sample Buffer (1×final) was added to all samples. The 13.3 μL of each sample was loaded onto a Novex™ 16% Tricine SDS-Gel (Invitrogen™) 120 V were applied for 85 min. The gels were briefly rinsed with water before imaging. Cy2 fluorescence was imaged using the standard settings for Cy2 on a Typhoon™ imager (Amersham), while mBBrwas imaged using the standard settings for ethidium bromide on a ChemiDoc imager (Bio-Rad). The bands were analysed using Fiji (Image J, version 2.1.0/1.53c) built-in gel tool. Each individual lane was defined using the built-in tool and the intensity profile of each lane plotted. The line tool was used to define the background and band borders, all continuous intensity of the target band above the baseline was integrated using the wand tool. The standard curve was derived from a linear fit (Prism7, Graphpad) of the measured average band intensity (three replicates) measured for each concentration (0-5 nmol). The derived formula was used to determine the concentration of the detected peptides; the calculated values were corrected for variation using an internal V5 standard with known concentration. A calculated concentration was only given if the Cy2 control showed a visible signal, otherwise a value of 0 μg/L was given.

### Intact Protein and Cyclic Peptide Mass Spectrometry

ESI-MS analysis of proteins and macrocyclic (depsi-) peptides was performed using a Waters Xevo G2 mass spectrometer equipped with a modified nanoAcquity LC system. The samples were injected and separated on a BEH C4 UPLC column (1.7 μm; 1.0×100 mm; Waters) with a flowrate of 50 μL/min and a water/acetonitrile gradient from 2% vol/vol to 80% vol/vol (0.1% vol/vol formic acid) over 20 minutes or alternatively, desalted and injected directly manually. The eluted sample was directly interfaced via a Zspray electrospray ionization source with a hybrid quadrupole time-of-flight mass spectrometer (Waters). While using a cone voltage of 30 V, data were acquired in positive ion mode with a range from 300-2000 m/z. For peptides the raw spectra are shown, while protein spectra were deconvoluted using the MaxEnt1 function within MassLynx software (Waters)9, 10. We used GPMAW (Lighthouse Data) software11 to calculate the theoretical wild-type protein molecular weights and edited them manually to accommodate the molecular weights of ncAAs. For fragmentation of SanA2BA r.s. 6 the same experimental setup was used. A gradient of water/acetonitrile (A/B) was used to eluted the peptides (1% B for 10 min, to 50% B in 10 min, to 95% B in 2 min, 1 min 95%, 1 min 99% B, to 1% 6 min, flowrate of 50 μL/min), directly interfaced via a Zspray electrospray ionization source with a hybrid quadrupole time-of-flight mass spectrometer (Waters). The target ion (689.3 Da) was selected and fragmented (Collision energy: 6 eV), MS/MS spectra were collected over a m/z range of 50-2.000 m/z. The raw fragmentation spectrum was compared and assigned manually to the theoretical a, b and y series ions of the 5 possible linear sequences predicted by MS-Product (Baker, P. R. and Clauser, K. R. http://prospector.ucsf.edu.).

### Automated Identification of Orthogonal aaRS Active Site Pairs Against a Panel of Noncanonical Monomers

For automated detection of orthogonal pairs from raw data a computational script was employed. Briefly, for aaRS1-monomer1 to be considered a pair with aaRS2-monomer2, aaRS1 must be active with monomer1 and inactive with monomer2, while aaRS2 must be active with monomer2 but inactive with monomer1. After obtaining single codon sfGFP-3-TCG or sfGFP-3-TAG expression data in the presence of amino acids or hydroxy acids, the thresholds used for activity and inactivity were above 10000 RFU/OD600 and below RFU/OD600, respectively. aaRSs are grouped in the following way, so that pairs cannot be found with both aaRSs in the same group, group 1 (pyrrolysyl+N class): MmPylRS, MmPylRS(PheOH_6), MmPylRS(ArOH); group 2 (pyrrolysyl ΔN class A): 1R26PylRS, 1R26PyIRS(CbzK); group 3 (tyrosyl class): AfTyrRS(plF), AfTyrRS(pAzF). Data for F-OH, plF-OH, and NapA-OH with AfTyrRS(plF) and AfTyrRS(pAzF) were excluded from the analysis, since we demonstrated that these hydroxy acid substrates in the presence of these tyrosyl aaRSs led only to the incorporation of the amino acid and not the hydroxy acid. A custom script was written in Python that takes a matrix of raw data values and identifies tRNAsynthetase/substrate pairs. The script iterates over raw data points for each aaRS/substrate experiment and searches for another aaRS/substrate that abides by the activity and inactivity thresholds defined above; this script is available at https://github.com/JWChin-Lab. Using these criteria, 77 aaRS-substrate pairs were identified, consisting of 49 unique substrate pairings (FIG. 11, Table 1).

### Chemical Syntheses—General Methods

All chemicals and solvents were purchased from Merck, Alfa Aesar or Fisher Scientific and used without further purification. Qualitative analysis by thin layer chromatography (TLC) was performed on aluminium sheets coated with silica (Merck TLC 60F-254). The spots were visualized under short wavelength ultra-violet lamp (254 nm) or stained with basic, aqueous potassium permanganate, ethanolic ninhydrin or vanillin. Flash column chromatography was performed with specified solvent systems on silica gel 60 (mesh 230-400). MPLC was performed on a Grace Reveleris X2, equipped with a C18, 120 g, 40 μm prepacked column. LC-MS analysis was performed on Agilent 1260 machine. The solvents used consisted of 0.2% formic acid in water (buffer A) and 0.2% formic acid in acetonitrile (buffer B). LC was performed using Agilent EC-C18 Poroshell 120 (50×3 mm, 2.7 μm) and monitored using variable wavelengths.

Mass spectrometry analysis following LC was carried out in multimode ESI and APCI on a 6130 Quadrupole spectrometer and recorded in both positive and negative ion modes. NMR analysis was carried out on a Bruker 400 MHz instrument. All reported chemical shifts (δ) relative to TMS were referenced to the residual protons in deuterated solvents used: d1—chloroform (1H δ=7.26 ppm, 13C δ=77.16 ppm), d6—dimethylsulfoxide (1H δ=2.49 ppm, 13C δ=39.52 ppm), D2O (1H δ=4.70). APT or two-dimensional experiments (COSY, HSQC) were always performed to provide additional information used for analysis where needed. Coupling constants are given in Hz and described as: singlet—s, doublet—d, triplet—t, quartet—q, broad singlet—br, multiplet—m, doublet of doublets—dd, etc. and combinations thereof.

### Synthesis of Hydroxy Acids

Hydroxy acids BocK-OH, AllocK-OH and CbzK-OH were all synthesised in accordance with published literature protocols⁵⁴

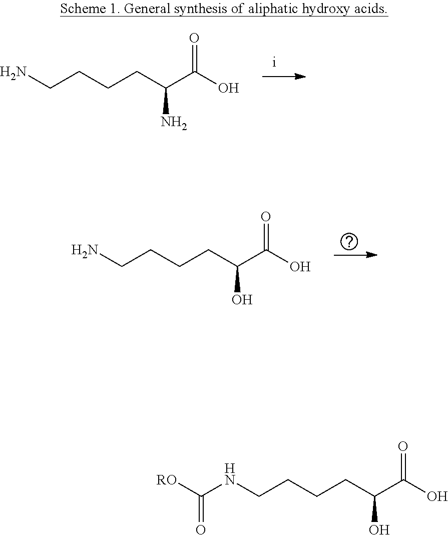

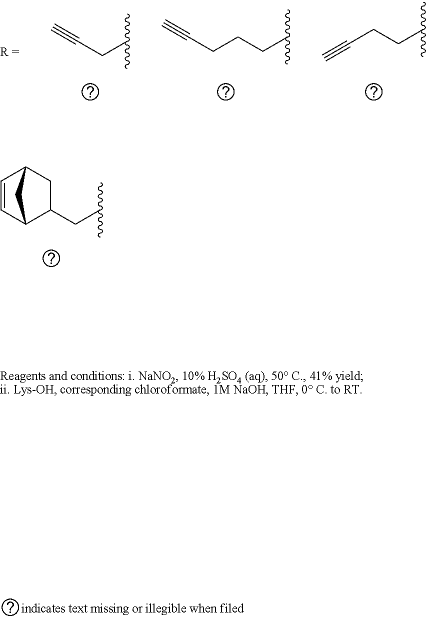

i. (S)-6-Amino-2-hydroxyhexanoic acid

A solution of (L)-lysine (10 g, 54.8 mmol, 1 eq) in 10% H2SO4 (110 mL) was heated to 50° C. and stirred. NaNO2 (12.8 g, 186.2 mmol, 3.4 eq) was dissolved in H2O (40 mL), and then added to the lysine solution dropwise over 1 hr. The reaction was stirred at 50° C. for 3 hrs. The reaction was quenched by the addition of urea (21 g in 60 mL H2O). The resultant solution was neutralised to pH 7 with the addition 1M NaOH, then acidified to pH 3 with formic acid. H2O and excess formic acid were removed under reduced pressure to concentrate the crude mixture to approximately 50 mL. Amberlite® IR120 hydrogen form strongly acidic resin (100 g, Fluka) was charged to a glass column and first washed with H2O to constant pH˜7, then with 1M NH4OH (200 mL), then with H2O to constant pH˜7, then with 1M HCl (200 mL) and finally with H2O to constant pH˜5. The crude hydroxy acid solution (˜50 mL) was loaded to the column and allowed to equilibrate at RT for 5 min. The flow through was collected and the resin washed with H2O to constant pH 5. The product was then eluted with 1M NH4OH (200 mL) and concentrated to give a yellow oil. This oil was dissolved in hot MeOH and allowed to cool to RT, which resulted in a precipitate that was collected by filtration and dried under reduced pressure. This gave the desired product (S)-6-amino-2-hydroxyhexanoic acid as a colourless solid (3.3 g, 41% yield). 1H NMR analysis δH (400 MHz, D2O) 3.98 (dd, J=6.8, 4.5 Hz, 1H), 2.94 (t, J=7.5 Hz, 2H), 1.83-1.52 (m, 4H), 1.50-1.19 (m, 2H). LRMS m/z (ES+) 148 [M+H]+.

ii. General Protocol for the Synthesis of AlynK-OH, ButK-OH, PenK-OH and NorK-OH

Typically (S)-6-amino-2-hydroxyhexanoic acid (250 mg, 1.7 mmol, 1.2 eq) was dissolved in 1M NaOHaq (2 mL) and THE (1 mL) and the solution stirred and cooled to 0° C. The corresponding chloroformate (1 eq) was added dropwise over 5 min and the reaction allow to warm to RT and stir for an additional 1 hr. After this time the reaction was diluted with 1M HCl (20 mL) and extracted with ethyl acetate (3×20 mL). The combined organic layers were dried over Na2SO4, filtered and concentrated. The crude hydroxy acids were typically purified by silica gel column chromatography eluting with ethyl acetate, hexane and acetic acid (50:48:2). This gave pure hydroxy acids as colourless oils. AlynK-OH (285 mg, 89% yield), LRMS m/z (ES−) 228 [M−H]−; ButK-OH (307 mg, 90% yield), LRMS m/z (ES−) 242 [M−H]−; PenK-OH (325 mg, 90% yield), LRMS m/z (ES−) 256 [M−H]− and NorK-OH (367 mg, 88% yield) LRMS m/z (ES−) 296 [M−H]−. For ButK-OH, PenK-OH and NorK-OH the chloroformates were pre-made by the addition of the corresponding alcohols (1.4 mmol, 1 eq; 3-Butyn-1-ol Merck—130850, 4-Pentyn-1-ol Merck—302481 and 5-Norbornene-2-methanol Merck—248533 mixture of endo and exo) in THF (1.5 mL) to a solution of phosgene (737 μL, 1.4 mmol, 1 eq, 20% in toluene) at 0° C. via syringe pump over 1 hr. The resulting solution was used directly without further purification.

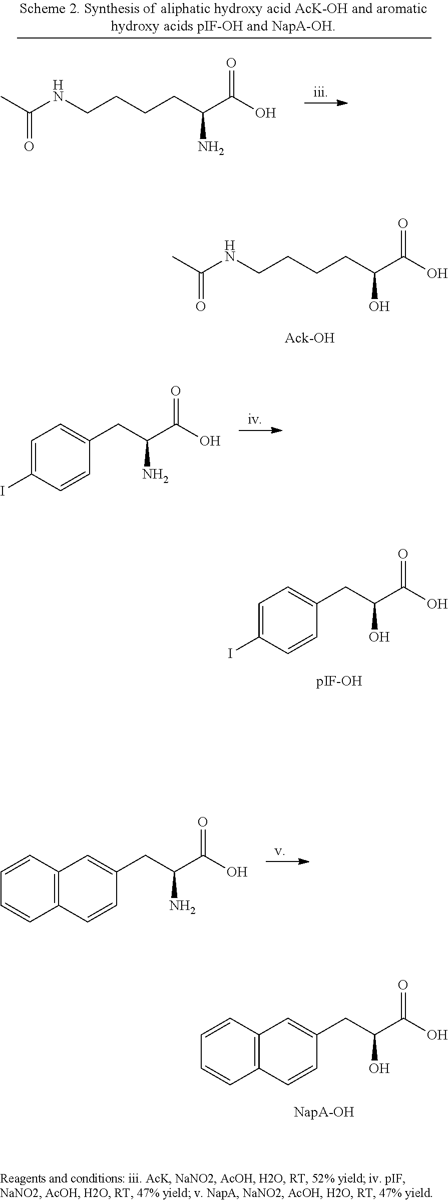 [SMILES: CC(=O)NCCCC[C@H](N)C(=O)O ; CC(=O)NCCCC[C@H](O)C(=O)O ; N[C@@H](Cc1ccc(I)cc1)C(=O)O ; O=C(O)[C@@H](O)Cc1ccc(I)cc1 ; N[C@@H](Cc1ccc2ccccc2c1)C(=O)O ; O=C(O)[C@@H](O)Cc1ccc2ccccc2c1]

iii. (S)-6-Acetamido-2-hydroxyhexanoic acid (Ack-OH)

A solution of NE-Acetyl-L-lysine (5.0 g, 26.6 mmol, 1 eq) in AcOH (10 mL) and H2O (40 mL) was stirred at RT. NaNO2 (9.2 g, 132.8 mmol, 5 eq) in H2O (25 mL) was then added to the amino acid solution dropwise over 30 min. The reaction was stirred at RT overnight. 1M HCl (50 mL) was added and mixture was extracted with ethyl acetate (3×50 mL). The combined organic layers were dried over Na2SO4, filtered and concentrated. The crude hydroxy acid was purified by C18 reverse phase MPLC eluting with H2O/acetonitrile (95/5 to 5/95 over 60 min). The combined product fractions were concentrated to dryness to give pure AcK-OH (2.6 g, 52% yield) as a colourless oil, 1H NMR analysis δH (400 MHz, D2O) 4.22 (dd, J=7.6, 4.5 Hz, 1H), 3.11 (t, J=6.7 Hz, 2H), 1.90 (s, 3H), 1.83-1.58 (m, 2H), 1.56-1.23 (m, 4H). LRMS m/z (ES−) 188 [M−H]−.

iv. (S)-2-hydroxy-3-(4-iodophenyl)propanoic acid (plF-OH)

A solution of 4-iodo-L-phenylalanine (2.0 g, 6.9 mmol, 1 eq) in AcOH (3 mL) and H2O (12 mL) was stirred at RT. NaNO2 (2.4 g, 34.4 mmol, 5 eq) in H2O (7 mL) was then added to the amino acid solution dropwise over 10 min. The reaction was stirred at RT overnight. 1M HCl (20 mL) was added and mixture was extracted with ethyl acetate (3×20 mL). The combined organic layers were dried over Na2SO4, filtered and concentrated. The crude hydroxy acid was purified by purified by silica gel column chromatography eluting with ethyl acetate, hexane and acetic acid (50:48:2). The combined product fractions were concentrated to dryness to give pure plF-OH (957 mg, 47% yield) as a colourless solid, LRMS m/z (ES−) 291 [M−H]−. v. (S)-2-hydroxy-3-(naphthalen-2-yl)propanoic acid (NapA-OH) A solution of 3-(2-naphthyl)-L-alanine (215 mg, 1.0 mmol, 1 eq) in AcOH (1.8 mL) and H2O (3.4 mL) was stirred at RT. NaNO2 (578 mg, 8.4 mmol, 8.4 eq) in H2O (2 mL) was then added to the amino acid solution dropwise over 5 min. The reaction was stirred at RT overnight. 1M HCl (20 mL) was added and mixture was extracted with ethyl acetate (3×20 mL). The combined organic layers were dried over Na2SO4, filtered and concentrated. The crude hydroxy acid was purified by purified by silica gel column chromatography eluting with ethyl acetate, hexane and acetic acid (50:48:2). The combined product fractions were concentrated to dryness to give pure NapA-OH (102 mg, 47% yield) as a colourless solid, 1H NMR analysis δH (400 MHz, CD3CN) 8.12-7.67 (m, 3H), 7.64-7.35 (m, 2H), 4.49 (dd, J=7.9, 4.2 Hz, 1H), 3.29 (dd, J=14.0, 4.3 Hz, 1H), 3.08 (dd, J=14.0, 7.8 Hz, 1H). LRMS m/z (ES−) 215 [M−H]⁻

TABLE 1
Mutually orthogonal aaRS/tRNA pairs for encoding different combinations
of two distinct non-canonical monomers. Some monomer combinations
may be encoded by several aaRS/tRNA combinations with varying
efficiency; all efficient combinations are listed.

| Monomers | aaRSs |
|---|---|
| AcK-OH - CbzK | MmPylRS - 1R26PylRS(CbzK) |
| AcK-OH - pAzF | MmPylRS - AfTyrRS(pAzF) |
| AcK-OH - pIF | MmPylRS - AfTyrRS(pIF) |
| AlkynK - CbzK | MmPylRS - 1R26PylRS(CbzK) |
| AlkynK - pAzF | MmPylRS -AfTyrRS(pAzF) |
| | 1R26PylRS-AfTyrRS(pAzF) |
| AlkynK - pIF | MmPylRS - AfTyrRS(pIF) |
| | 1R26PylRS - AfTyrRS(pIF) |
| AlkynK-OH - pAzF | MmPylRS - AfTyrRS(pAzF) |
| | 1R26PylRS - AfTyrRS(pAzF) |
| AlkynK-OH - pIF | MmPylRS - AfTyrRS(pIF) |
| | 1R26PylRS - AfTyrRS(pIF) |
| AllocK - CbzK | MmPylRS - 1R26PylRS(CbzK) |
| AllocK - pAzF | MmPylRS - AfTyrRS(pAzF) |
| | 1R26PylRS - AfTyrRS(pAzF) |
| AllocK - pIF | MmPylRS - AfTyrRS(pIF) |
| | 1R26PylRS - AfTyrRS(pIF) |
| AllocK-OH - pAzF | MmPylRS - AfTyrRS(pAzF) |
| | 1R26PylRS - AfTyrRS(pAzF) |
| AllocK-OH - pIF | MmPylRS - AfTyrRS(pIF) |
| | 1R26PylRS - AfTyrRS(pIF) |
| BocK-OH - pAzF | MmPylRS - AfTyrRS(pAzF) |
| BocK-OH - pIF | MmPylRS - AfTyrRS(pIF) |
| ButK-OH - pAzF | MmPylRS - AfTyrRS(pAzF) |
| | 1R26PylRS - AfTyrRS(pAzF) |
| ButK-OH - pIF | MmPylRS - AfTyrRS(pIF) |
| | 1R26PylRS - AfTyrRS(pIF) |
| CbzK - pAzF | 1R26PylRS(CbzK) - AfTyrRS(pAzF) |
| CbzK - pIF | 1R26PylRS(CbzK) - AfTyrRS(pIF) |
| CbzK-OH - pAzF | MmPylRS - AfTyrRS(pAzF) |
| CbzK-OH - pIF | MmPylRS - AfTyrRS(pIF) |
| F-OH - AlkynK | MmPylRS(PheOH_6) - 1R26PylRS |
| | MmPylRS(ArOH) - 1R26PylRS |
| F-OH - AlkynK-OH | MmPylRS(PheOH_6) - 1R26PylRS |
| | MmPylRS(ArOH) - 1R26PylRS |
| F-OH - AllocK | MmPylRS(PheOH_6) - 1R26PylRS |
| | MmPylRS(ArOH) - 1R26PylRS |
| F-OH - AllocK-OH | MmPylRS(PheOH_6) - 1R26PylRS |
| | MmPylRS(ArOH) - 1R26PylRS |
| F-OH - ButK-OH | MmPylRS(PheOH_6) - 1R26PylRS |
| | MmPylRS(ArOH) - 1R26PylRS |
| F-OH - CbzK | MmPylRS(PheOH_6) - 1R26PylRS(CbzK) |
| | MmPylRS(ArOH) -1R26PylRS(CbzK)F-OH - NorK-OH |
| | MmPylRS(PheOH_6) - 1R26PylRS(CbzK) |
| | MmPylRS(ArOH) - 1R26PylRS(CbzK) |
| F-OH - PenK-OH | MmPylRS(PheOH_6) - 1R26PylRS, |
| | MmPylRS(PheOH_6) - 1R26PylRS(CbzK) |
| | MmPylRS(ArOH) - 1R26PylRS(CbzK) |
| | MmPylRS(ArOH) - 1R26PylRS |
| NapA-OH - AlkynK | MmPylRS(ArOH) - 1R26PylRS |
| NapA-OH - AlkynK-OH | MmPylRS(ArOH) - 1R26PylRS |
| NapA-OH - AllocK | MmPylRS(ArOH) - 1R26PylRS |
| NapA-OH - AllocK-OH | MmPylRS(ArOH) - 1R26PylRS |
| NapA-OH - ButK-OH | MmPylRS(ArOH) -1R26PylRS |
| NapA-OH - CbzK | MmPylRS(ArOH) - 1R26PylRS(CbzK) |
| NapA-OH - NorK-OH | MmPylRS(ArOH) - 1R26PylRS(CbzK) |
| NapA-OH - PenK-OH | MmPylRS(ArOH) -1R26PylRS |
| | MmPylRS(ArOH) - 1R26PylRS(CbzK) |
| NorK-OH - pAzF | MmPylRS - AfTyrRS(pAzF) |
| | 1R26PylRS (CbzK) - AfTyrRS(pAzF) |
| NorK-OH - pIF | MmPylRS - AfTyrRS(pIF) |
| | 1R26PylRS(CbzK) - AfTyrRS(pIF) |
| PenK-OH - pAzF | MmPylRS - AfTyrRS(pAzF) |
| | 1R26PylRS - AfTyrRS(pAzF) |
| | 1R26PylRS(CbzK) - AfTyrRS(pAzF) |
| PenK-OH - pIF | MmPylRS - AfTyrRS(pIF) |
| | 1R26PylRS - AfTyrRS(pIF) |
| | 1R26PylRS(CbzK) - AfTyrRS(pIF) |
| pIF-OH - AlkynK | MmPylRS(ArOH) - 1R26PylRS |
| pIF-OH - AlkynK-OH | MmPylRS(ArOH) - 1R26PylRS |
| pIF-OH - AllocK | MmPylRS(ArOH) - 1R26PylRS |
| pIF-OH - AllocK-OH | MmPylRS(ArOH) - 1R26PylRS |
| pIF-OH - ButK-OH | MmPylRS(ArOH) - 1R26PylRS |
| pIF-OH - CbzK | MmPylRS(ArOH) - 1R26PylRS(CbzK) |
| pIF-OH - NorK-OH | MmPylRS(ArOH) - 1R26PylRS(CbzK) |
| pIF-OH - PenK-OH | MmPylRS(ArOH) - 1R26PylRS, |
| | MmPylRS(ArOH) - 1R26PylRS(CbzK) |

TABLE 2

| | |
|---|---|
| Plasmids | |
| pMB1_MmPylRS_MmtRNA-PylTOpt(CUA) | Single aaRS/tRNA plasmid |
| | with MmPylRS wt and |
| | MmtRNA-opt to incorporate |
| | pyrrolysine derivatives into |
| | the TAG codon |
| pMB1_MmPylRS(PheOH_1)_MmtRNA-PylTOpt(CUA) | Single aaRS/tRNA plasmid |
| | with MmPylRS(M300S- |
| | A302H-N346G) to |
| | incorporate L-phenyllactic |
| | acid into the TAG codon |
| pMB1_MmPylRS(PheOH_3)_MmtRNA-PylTOpt(CUA) | Single aaRS/tRNA plasmid |
| | with MmPylRS(M300S- |
| | L301P-A302H-M344V- |
| | N346A) to incorporate L- |
| | phenyllactic acid into the |
| | TAG codon |
| pMB1_MmPylRS(PheOH_6)_MmtRNA-PylTOpt(CUA) | Single aaRS/tRNA plasmid |
| | with MmPylRS(M300S- |
| | A302H-M344L-N346A) to |
| | incorporate L-phenyllactic |
| | acid into the TAG codon |
| pMB1_MmPylRS(ArOH)_MmtRNA-PylTOpt(CUA) | Single aaRS/tRNA plasmid |
| | with MmPylRS(M300S- |
| | A302H-M344L-N346A- |
| | C348S-V401L-W417T) to |
| | incorporate aromatic |
| | hydroxy acids into the TAG |
| | codon |
| pMB1_MmPylRS_MmtRNA-Pyl(CGA) | Recoded single aaRS/tRNA |
| | plasmid with MmPylRS wt |
| | and MmtRNA-Pyl-opt with |
| | the ASL from the serU gene |
| | (CGA anticodon) to |
| | incorporate pyrrolysine |
| | derivatives into the TCG |
| | codon |
| pMB1_1R26PylRS_AlvtRNA-ΔNPyl(8)(CUA) | Recoded single aaRS/tRNA |
| | plasmid with 1R26PylRS wt |
| | and AlvtRNA- |
| | ΔNPyl(8)(CUA) to |
| | incorporate pyrroysine |
| | derivatives into the TAG |
| | codon |
| pMB1_1R26PylRS(CbzK)_AlvtRNA-ΔNPyl(8)(CUA) | Recoded single aaRS/tRNA |
| | plasmid with |
| | 1R26PylRS(Y126G-M129L) |
| | (Ref 18) and AlvtRNA- |
| | ΔNPyl(8)(CUA) to |
| | incorporate CbzK or CbzK- |
| | OH into the TAG codon |
| pMB1_AfTyrRS(pIF)_AftRNA-Tyr(A01)(CUA) | Recoded single aaRS/tRNA |
| | plasmid with AfTyrRS(Y36I- |
| | L69M-H74L-Q116E-D165T- |
| | I166G) and AftRNA- |
| | Tyr(A01)(CUA) to |
| | incorporate p-I-Phe into |
| | the TAG codon |
| pMB1_AfTyrRS(pAzF)_AftRNA-Tyr(A01)(CUA) | Recoded single aaRS/tRNA |
| | plasmid with |
| | AfTyrRS(Y36T-H74L- |
| | Q116E-D165T-I166G- |
| | N190K) and AftRNA- |
| | Tyr(A01)(CUA) to |
| | incorporate p-Az-Phe into |
| | the TAG codon |
| pMB1_MmPylRS(PheOH_6)_tRNA-Pyl(CGA) | Recoded single aaRS/tRNA |
| | plasmid with |
| | MmPylRS(M300S-A302H- |
| | M344L-N346A) to |
| | incorporate L-phenyllactic |
| | acid into the TCG codon |
| pMB1_MmPylRS(ArOH)_tRNA-Pyl(CGA) | Recoded single aaRS/tRNA |
| | plasmid with |
| | MmPylRS(M300S-A302H- |
| | M344L-N346A-C348S- |
| | V401L-W417T) to |
| | incorporate aromatic |
| | hydroxy acids into the TAG |
| | codon |
| pMB1_MmPylRS(AzK)_MmtRNA-Pyl(CGA) | Recoded single aaRS/tRNA |
| | plasmid with MmPylRS- |
| | L309A-C348V-Y384F (Ref |
| | 16) and MmtRNA-Pyl-opt |
| | with the ASL from the serU |
| | gene (CGA anticodon) to |
| | incorporate pyrrolysine |
| | derivatives into the TCG |
| | codon |
| pMB1_MmPylRS(NorK)_MmtRNA-Pyl(CGA) | Recoded single aaRS/tRNA |
| | plasmid with MmPylRS- |
| | Y306G-Y384F-I405R (Ref |
| | 35) and MmtRNA-Pyl-opt |
| | with the ASL from the serU |
| | gene (CGA anticodon) to |
| | incorporate pyrrolysine |
| | derivatives into the TCG |
| | codon |
| pMB1_MmPylRS(AcK)_MmtRNA-Pyl(CGA) | Recoded single aaRS/tRNA |
| | plasmid with MmPylRS- |
| | L301M-Y306L-L309A- |
| | C348F-L367M (Ref 36) and |
| | MmtRNA-Pyl-opt with the |
| | ASL from the serU gene |
| | (CGA anticodon) to |
| | incorporate pyrrolysine |
| | derivatives into the TCG |
| | codon |
| pMB1_1R26PylRS(AzbK)_AlvtRNA-ΔNPyl(8)(CUA) | Recoded single aaRS/tRNA |
| | plasmid with 1R26PylRS- |
| | M129A-Y206F and |
| | AlvtRNA-ΔNPyl(8)(CUA) to |
| | incorporate pyrrolysyine |
| | derivatives into the TAG |
| | codon |
| pMB1_MmPylRS_AfTyrRS(pIF)_MmtRNA-Pyl(CGA)- | Recoded double |
| AftRNA-Tyr(A01)(CUA) | aaRS/tRNA plasmid with |
| | MmPylRS and |
| | AfTyrRS(Y36I-L69M-H74L- |
| | Q116E-D165T-I166G) with |
| | MmtRNA-Pyl-opt(CGA) and |
| | AftRNA-Tyr(A01)(CUA) |
| | tRNAs to incorporate |
| | pyrrolysine derivatives into |
| | the TCG codon and p-I-Phe |
| | into the TAG codon; used |
| | for reassignment scheme 1 |
| | and 4. |
| pMB1_MmPylRS_1R26PylRS(CbzK)_MmtRNA- | Recoded double |
| Pyl(CGA)_AlvtRNA-ΔNPyl(8)(CUA) | aaRS/tRNA plasmid with |
| | MmPylRS and |
| | 1R26PylRS(Y126G-M128L) |
| | with MmtRNA-Pyl-opt(CGA) |
| | and AlvtRNA- |
| | ΔNPyl(8)(CUA) tRNAs to |
| | incorporate AllocK into the |
| | TCG codon and CbzK into |
| | the TAG codon; used for |
| | reassignment scheme 2 and |
| | 5. |
| pMB1_MmPylRS_AfTyrRS(pAzF)_MmtRNA-Pyl(CGA)- | Recoded double |
| AftRNA-Tyr(A01)(CUA) | aaRS/tRNA plasmid with |
| | MmPylRS and |
| | AfTyrRS(Y36T-H74L- |
| | Q116E-D165T-I166G- |
| | N190K) with MmtRNA-Pyl- |
| | opt(CGA) and AftRNA- |
| | Tyr(A01)(CUA) tRNAs to |
| | incorporate BocK into the |
| | TCG codon and p-Azido- |
| | Phe into the TAG codon; |
| | used for reassignment |
| | scheme 3 and 6. |
| pMB1_MmPylRS(PheOH_6)_1R26PylRS(CbzK)_MmtRNA- | Recoded double |
| Pyl(CGA)_AlvtRNA-ΔNPyl(8)(CUA) | aaRS/tRNA plasmid with |
| | MmPylRS(M300S-A302H- |
| | M344L-N346A) and |
| | 1R26PylRS(Y126G-M128L) |
| | with MmtRNA-Pyl-opt(CGA) |
| | and AlvtRNA- |
| | ΔNPyl(8)(CUA) tRNAs to |
| | incorporate L-phenyllactic |
| | acid into the TCG codon |
| | and CbzK into the TAG |
| | codon; used for |
| | reassignment scheme 7 |
| pMB1_MmPylRS(ArOH)_1R26PylRS_MmtRNA- | Recoded double |
| Pyl(CGA)_AlvtRNA-ΔNPyl(8)(CUA) | aaRS/tRNA plasmid with |
| | MmPylRS(M300S-A302H- |
| | M344L-N346A-C348S- |
| | V401L-W417T) and |
| | 1R26PylRS with MmtRNA- |
| | Pyl-opt(CGA) and AlvtRNA- |
| | ΔNPyl(8)(CUA) tRNAs to |
| | incorporate aromatic |
| | hydroxy acids into the TCG |
| | codon and AllocK into the |
| | TAG codon; used for |
| | reassignment scheme 8 |
| Reporters (examples below) | |
| pBAD_SUMO-SanA1AB-GyrA-CBD | Recoded yeast SUMO |
| | followed by the SanA1AB |
| | sequence and a GyrA-CBD |
| | fusion protein. |
| pBAD_SUMO-SanA2AB-GyrA-CBD | Recoded yeast SUMO |
| | followed by the SanA2AB |
| | sequence and a GyrA-CBD |
| | fusion protein. |
| pBAD_SUMO-SanA3AB-GyrA-CBD | Recoded yeast SUMO |
| | followed by the SanA3AB |
| | sequence and a GyrA-CBD |
| | fusion protein. |
| pBAD_SUMO-SanA4AB-GyrA-CBD | Recoded yeast SUMO |
| | followed by the SanA4AB |
| | sequence and a GyrA-CBD |
| | fusion protein. |
| pBAD_SUMO-YMAB-GyrA-CBD | Recoded yeast SUMO |
| | followed by the YMAB |
| | sequence and a GyrA-CBD |
| | fusion protein. |
| pBAD_SUMO-SanA1BA-GyrA-CBD | Recoded yeast SUMO |
| | followed by the SanA1BA |
| | sequence and a GyrA-CBD |
| | fusion protein. |
| pBAD_SUMO-SanA2BA-GyrA-CBD | Recoded yeast SUMO |
| | followed by the SanA2BA |
| | sequence and a GyrA-CBD |
| | fusion protein. |
| pBAD_SUMO-SanA3BA-GyrA-CBD | Recoded yeast SUMO |
| | followed by the SanA3BA |
| | sequence and a GyrA-CBD |
| | fusion protein. |
| pBAD_SUMO-SanA4BA-GyrA-CBD | Recoded yeast SUMO |
| | followed by the SanA4BA |
| | sequence and a GyrA-CBD |
| | fusion protein. |
| pBAD_SUMO-YMBA-GyrA-CBD | Recoded yeast SUMO |
| | followed by the YMBA |
| | sequence and a GyrA-CBD |
| | fusion protein. |
| Helper | Contains lambda-red |
| | recombination components, |
| | Cas9, and tracrRNA |

| | | | SEQ | | SEQ |
|---|---|---|---|---|---|
| Target | | | ID | | ID |
| gene | Purpose | Oligo F (5′-3′) | NO: | Oligo R (5′ - 3′) | NO: |
| aspC | Integrate pheS*- | tcaggcaatgttgcgtttgt | 1 | agtaatagttggaattttgt | 2 |
| | HygR | catcagtctcagcccgcttt | | aaatctcccgttaccctgat | |
| | | tcagcgggcttcattgtttt | | agcggacttcccttctgtaa | |
| | | taatgcTTATTCCTTTGCCC | | ccatagcacgtgttgacaat | |
| | | TCGGACGAGTGCTGG | | taatcatcggcatag | |
| aspC | 5′ region | tattgcaggggacgcgtagg | 3 | cccttctgtaaccatgcatt | 4 |
| | amplification | cctgata | | aaaaacaatgaagcccgctg | |
| | | | | aaaagcg | |
| aspC | 3′ region | gcttcattgtttttaatgca | 5 | ctcacgtottgcaaaaacag | 6 |
| | amplification | tggttacagaagggaagtcc | | cctgcgt | |
| | | gctatcagg | | | |
| aspC | Repair template | tattgcaggggacgcgtagg | 7 | ctcacgtcttgcaaaaacag | 8 |
| | for clean aspC | cctgata | | cctgcgt | |
| | KO | | | | |
| aspC | Genotype aspC | cctggatttctggcaaagtg | 9 | atcgtttaccagttctaata | 10 |
| | genomic region | cgctt | | gcacacctctttgt | |
| tyrB | Integrate pheS*- | tgaacatccactcgatcttc | 11 | gagtgaaagcgacgacagtt | 12 |
| | HygR | gccttcttccggtttattgt | | taatttcactgcaggctggg | |
| | | gttttaaccacctgcccgta | | tagctccagcctgctttcct | |
| | | aacctgTTATTCCTTTGCCC | | gcaagcacgtgttgacaatt | |
| | | TCGGACGAGTGCTGG | | aatcatcggcatagt | |
| tyrB | 5′ region | gcctctggcggatgtacgtt | 13 | gcctgctttcctgcacaggt | 14 |
| | amplification | tgtcatgag | | ttacgggcaggtggttaaaa | |
| | | | | cac | |
| tyrB | 3′ region | gcccgtaaacctgtgcagga | 15 | tccagataattgggataagc | 16 |
| | amplification | aagcaggctggagctaccca | | ttaacaataaaaga | |
| tyrB | Repair template | gcctctggcggatgtacgtt | 17 | tccagataattgggataagc | 18 |
| | for clean tyrB | tgtcatgag | | ttaacaataaaaga | |
| | KC | | | | |
| tyrB | Genotype tyrB | cggatgcggcgtgaatgcct | 19 | ggcaaagcatattcttgagg | 20 |
| | genomic region | tatcc | | atatagaaatattc | |
| SUMO-gyrA | Qickchange | GTGGTTGCCTGtagCTGTCG | 21 | tgcaCGACAGctaCAGGCAA | 22 |
| | SanA1AB | tgcatcacgggagatgcact | | CCACCAATCTGTTCTCTGT | |
| | | ag | | | |
| SUMO-gyrA | Qickchange | GTGGTTGCTAGCTGgttTCG | 23 | tgcaCGAaacCAGCTAGCAA | 24 |
| | SanABA | tgcatcacgggagatgcact | | CCACCAATCTGTTCTCTGT | |
| | | ag | | | |
| SUMO-gyrA | SUMO/GyrA | ATGCACACCTGCAAGgcatc | 25 | ATGCACACCTGCTTACCACC | 26 |
| | Aral backbone | acgggagatgcact | | AATCTGTTCTCTGTGAGC | |
| SUMO-gyrA | SanA1BA | GGTGGTTGCCTATCGCTGTA | 27 | gatgcaCTACAGCGATAGGC | 28 |
| | annealed oligos | Gtg | | AAC | |
| SUMO-gyrA | SanA3AB | GGTGGTTGCCTATCGCTGTA | 29 | gatgcaAAGCTACAGCGATA | 30 |
| | annealed oligos | GCTTtg | | GGCAAC | |
| SUMO-gyrA | SanA3BA | GGTGGTTGCCTATAGCTGTC | 31 | gatgcaAAGCGACAGCTATA | 32 |
| | annealed oligos | GCTTtg | | GGCAAC | |
| SUMO-gyrA | SanA4AB | GGTGGTTGCCTATCGCTGTA | 33 | gatgcaAACCTACAGCGATA | 34 |
| | annealed oligos | GGTTtg | | GGCAAC | |
| SUMO-gyrA | SanA4BA | GGTGGTTGCCTATAGCTGTC | 35 | gatgcaAACCGACAGCTATA | 36 |
| | annealed oligos | GGTTtg | | GGCAAC | |
| SUMO-gyrA | YMBA annealed | GGTGGTTGCTAGacgACATC | 37 | gatgcaagccgcCGATGTcg | 38 |
| | oligos | Ggcggcttg | | tCTAGCAAC | |
| SUMO-gyrA | YMAB annealed | GGTGGTTGCTCGacgACATA | 39 | gatgcaagccgcCTATGTcg | 40 |
| | oligos | Ggcggcttg | | tCGAGCAAC | |
| MmPyIRS | PheOH Library | gatcGGTCTCtgcgtccgSV | 41 | GATCggtctcGACGCAGGCA | 42 |
| wt | creation | Tnnknnkccgaacctgtaca | | GAAGTTTTTGTCAACACGG | |
| | (position 1) Set 1 | actacctgcgtaaactgg | | | |
| | (30%) | | | | |
| MmPyIRS | PheOH Library | gatcGGTCTCtgcgtccgSV | 43 | GATCggtctcGACGCAGGCA | 44 |
| wt | creation | Tnnknnkccgaacctgtaca | | GAAGTTTTTGTCAACACGG | |
| | (position 1) Set 2 | actacctgcgtaaactgg | | | |
| | (30%) | | | | |
| MmPyIRS | PheOH Library | gatcGGTCTCtgcgtccgVW | 45 | GATCggtctcGACGCAGGCA | 46 |
| wt | creation | Gnnknnkccgaacctgtaca | | GAAGTTTTTGTCAACACGG | |
| | (position 1) Set 3 | actacctgcgtaaactgg | | | |
| | (30%) | | | | |
| MmPyIRS | PheOH Library | gatcGGTCTCtgcgtccgTG | 47 | GATCggtctcGACGCAGGCA | 48 |
| wt | creation | Snnknnkccgaacctgtaca | | GAAGTTTTTGTCAACACGG | |
| | (position 1) Set 4 | actacctgcgtaaactgg | | | |
| | (10%) | | | | |
| MmPyIRS | PheOH Library | GATCggtctcCTGCCAGATG | 49 | GATCggtctcTGGCAGAAAB | 50 |
| wt | creation | GGTTCTGGTTGCACCCGTGA | | ScagABSggtgaattcttcc | |
| | (position 2) Set 1 | AAACC | | aggtgttctttaCC | |
| | (9%) | | | | |
| MmPyIRS | PheOH Library | GATCggtctcCTGCCAGATG | 51 | GATCggtctcTGGCAGAAAB | 52 |
| wt | creation | GGTTCTGGTTGCACCCGTGA | | ScagGDWggtgaattcttcc | |
| | (position 2) Set 2 | AAACC | | aggtgttctttacc | |
| | (9%) | | | | |
| MmPyIRS | PheOH Library | GATCggtctcCTGCCAGATG | 53 | GATCggtctcTGGCAGAAAB | 54 |
| wt | creation | GGTTCTGGTTGCACCCGTGA | | ScagCWBggtgaattcttcc | |
| | (position 2) Set 3 | AAACC | | aggtgttctttacc | |
| | (9%) | | | | |
| MmPyIRS | PheOH Library | GATCggtctcCTGCCAGATG | 55 | GATCggtctcTGGCAGAAAB | 56 |
| wt | creation | GGTTCTGGTTGCACCCGTGA | | ScagSCAggtgaattcttcc | |
| | (position 2) Set 4 | AAACC | | aggtgttctttacc | |
| | (3%) | | | | |
| MmPyIRS | PheOH Library | GATCggtctcCTGCCAGATG | 57 | GATCggtctcTGGCAGAAGD | 58 |
| wt | creation | GGTTCTGGTTGCACCCGTGA | | WcagABSggtgaattcttcc | |
| | (position 2) Set 5 | AAACC | | aggtgttctttacc | |
| | (9%) | | | | |
| MmPyIRS | PheOH Library | GATCggtctcCTGCCAGATG | 59 | GATCggtctcTGGCAGAAGD | 60 |
| wt | creation | GGTTCTGGTTGCACCCGTGA | | WcagGDWggtgaattcttcc | |
| | (position 2) Set 6 | AAACC | | aggtgttctttacc | |
| | (9%) | | | | |
| MmPyIRS | PheOH Library | GATCggtctcCTGCCAGATG | 61 | GATCggtctcTGGCAGAAGD | 62 |
| wt | creation | GGTTCTGGTTGCACCCGTGA | | WcagCWBggtgaattcttcc | |
| | (position 2) Set 7 | AAACC | | aggtgttctttacc | |
| | (9%) | | | | |
| MmPyIRS | PheOH Library | GATCggtctcCTGCCAGATG | 63 | GATCggtctcTGGCAGAAGD | 64 |
| wt | creation | GGTTCTGGTTGCACCCGTGA | | WcagSCAggtgaattcttcc | |
| | (position 2) Set 8 | AAACC | | aggtgttctttacc | |
| | (3%) | | | | |
| MmPyIRS | PheOH Library | GATCggtctcCTGCCAGATG | 65 | GATCggtctcTGGCAGAACW | 66 |
| wt | creation | GGTTCTGGTTGCACCCGTGA | | BcagABSggtgaattcttcc | |
| | (position 2) Set 9 | AAACC | | aggtgttctttacc | |
| | (9%) | | | | |
| MmPyIRS | PheOH Library | GATCggtctcCTGCCAGATG | 67 | GATCggtctcTGGCAGAACW | 68 |
| wt | creation | GGTTCTGGTTGCACCCGTGA | | BcagGDWggtgaattcttcc | |
| | (position 2) Set | AAACC | | aggtgttctttacc | |
| | 10 (9%) | | | | |
| MmPyIRS | PheOH Library | GATCggtctcCTGCCAGATG | 69 | GATCggtctcTGGCAGAACW | 70 |
| wt | creation | GGTTCTGGTTGCACCCGTGA | | BcagCWBggtgaattcttcc | |
| | (position 2) Set | AAACC | | aggtgttctttacc | |
| | 11 (9%) | | | | |
| MmPyIRS | PheOH Library | GATCggtctcCTGCCAGATG | 71 | GATCggtctcTGGCAGAACW | 72 |
| wt | creation | GGTTCTGGTTGCACCCGTGA | | BcagSCAggtgaattcttcc | |
| | (position 2) Set | AAACC | | aggtgttctttacc | |
| | 12 (3%) | | | | |
| MmPyIRS | PheOH Library | GATCggtctcCTGCCAGATG | 73 | GATCggtctcTGGCAGAASC | 74 |
| wt | creation | GGTTCTGGTTGCACCCGTGA | | AcagABSggtgaattcttcc | |
| | (position 2) Set | AAACC | | aggtgttctttaCC | |
| | 13 (3%) | | | | |
| MmPyIRS | PheOH Library | GATCggtctcCTGCCAGATG | 75 | GATCggtctcTGGCAGAASC | 76 |
| wt | creation | GGTTCTGGTTGCACCCGTGA | | AcagGDWggtgaattcttcc | |
| | (position 2) Set | AAACC | | aggtgttctttacc | |
| | 14 (3%) | | | | |
| MmPyIRS | PheOH Library | GATCggtctcCTGCCAGATG | 77 | GATCggtctcTGGCAGAASC | 78 |
| wt | creation | GGTTCTGGTTGCACCCGTGA | | AcagCWBggtgaattcttcc | |
| | (position 2) Set | AAACC | | aggtgttctttacc | |
| | 15 (3%) | | | | |
| MmPyIRS | PheOH Library | GATCggtctcCTGCCAGATG | 79 | GATCggtctcTGGCAGAASC | 80 |
| wt | creation | GGTTCTGGTTGCACCCGTGA | | AcagSCAggtgaattcttcc | |
| | (position 2) Set | AAACC | | aggtgttctttacc | |
| | 16 (1%) | | | | |
| MmPyIRS | NapAOH Library | GATGGGTTCTGGTTGCACCC | 81 | GGGTGCAACCAGAACCCATC | 82 |
| wt | creation | GTGAAAACCTGGAATCTATC | | TGMNNGAAaccCAGcatGGT | |
| | (position 1) on | ATCACCGAC | | GAATTCTTCC | |
| | PheOH-RS1 | | | | |
| MmPyIRS | NapAOH Library | GATGGGTTCTGGTTGCACCC | 83 | GGGTGCAACCAGAACCCATC | 84 |
| wt | creation | GTGAAAACCTGGAATCTATC | | TGMNNGAAaccCAGcacGGT | |
| | (position 1) on | ATCACCGAC | | GAATTCTTCC | |
| | PheOH-RS3 | | | | |
| MmPyIRS | NapAOH Library | GATGGGTTCTGGTTGCACCC | 85 | GGGTGCAACCAGAACCCATC | 86 |
| wt | creation | GTGAAAACCTGGAATCTATC | | TGMNNGAAagcCAGcagGGT | |
| | (position 1) on | ATCACCGAC | | GAATTCTTCC | |
| | PheOH-RS6 | | | | |
| MmPyIRS | NapAOH Library | CCGCTGGACCGTGAATGGGG | 87 | TACCCCATTCACGGTCCAGC | 88 |
| wt | creation | TATCGACAAACCGNNKATCG | | GGGATCGGACCAACMNNAGC | |
| | (position 2) on | GTGCTGGTTTCGGTCTGG | | AGAAGACAGTTCCAGGTCAC | |
| | PheOH-RS1, 3, | | | CG | |
| | and 6 | | | | |

### Example 1

### Combinatorially Encoding Non-Canonical Amino Acids in Macrocyclic Peptides

We designed ten genes encoding sequences inspired by the cyclic depsipeptides Sansalvamide A⁵⁵ and YM-254890⁵⁶ (FIG. 1a, FIG. 2). We placed TCG and TAG codons within the macrocycle-encoding genes to direct the incorporation of non-canonical monomers.

We encoded the genes as fusions to an N-terminal SUMO (small ubiquitin-like modifier) and a C-terminal GyrA intein-CBD domain, to enable excision and cyclization to generate the desired macrocycles⁵².

We encoded ncAAs (1-5) in response to the TCG and TAG codons in the macrocycle-encoding genes according to reassignment schemes (r.s.) 1-3. In each reassignment scheme monomer A is encoded in response to TCG and monomer B is encoded in response to TAG (FIG. 1a, b). The ncAAs introduced within each reassignment scheme were directed into the peptide using engineered mutually orthogonal aaRS/tRNA pairs; each aaRS/tRNA pair recognizes one ncAA and decodes either the TCG or TAG codon. In r.s. 1, monomer A is N-Alloc-L-lysine (1, AllocK) and is incorporated using the Methanosarcina mazei (Mm)PyIRS/MmtRNAPylCGA pair, and monomer B is p-lodo-phenylalanine (3, plF) and is incorporated using the Archeoglobus fulgidus (AI)TyrRS(plF)/AftRNATyr(A01)CUA pair. In r.s. 2, monomer A is AllocK, and is incorporated using the MmPylRS/MmtRNAPylCGA pair, and monomer B is N6-Carbobenzyloxy-L-lysine (4, CbzK), and is incorporated using the Methanomethylophilus sp. 1R26 (1R26)PyIRS(CbzK)/AlvtRNAΔNPyl(8)CUA pair. In r.s. 3, monomer A is Nε-((Prop-2-yn-1-yloxy)carbonyl)-L-lysine (2, AlkynK), and is incorporated using the MmPylRS/MmtRNAPylCGA pair, and monomer B is p-Azido-Phenylalanine (5, pAzF) and is incorporated using the AfTyrRS(pAzF)/AtRNATyr(A01)CUA pair.

We aimed to program the synthesis of 30 diverse non-natural macrocycles, by combining the ten macrocycle-encoding fusion genes and the 3 ncAA reassignment schemes. For each combination tested we aimed to express and purify the resulting fusion proteins from Syn61Δ3(ev5), and then to excise and cyclize the peptide, with Ulp1 (a C-terminal SUMO protease) in the presence of a suitable reductant, in vitro (FIG. 3). The resulting peptides were extracted, after a 16 h incubation, by chloroform/isopropanol extraction. For 25 of the combinations tested we produced and extracted the desired cyclic peptide, as judged by electrospray ionization mass spectrometry (ESI-MS) (FIG. 1c, d, e, FIG. 4).

For all cyclic peptides characterized by ESI-MS the precursor proteins were purified in good yield (1.4-7.3 mg per L of culture (FIG. 3)) and Ulp1-mediated cleavage led to disappearance of SUMO-peptide-GyrA-CBD and the appearance of GyrA-CBD on SDSPAGE (FIG. 3), consistent with peptide excision. The efficiency with which the excised peptides were extracted from the cleavage reaction for mass spectrometry was dependent on both their sequence and the solvent used for extraction (FIG. 5).

The variation in the intensity of the MS signal for extracted peptides (FIG. 1c, d, e, FIG. 4) is likely to result from differences in extracted yields or from differences in the ionization efficiencies of the extracted peptides in mass spectrometry.

Our results demonstrate that we can programme the combinatorial cell-based synthesis of diverse non-canonical amino acid containing macrocycles; within each macrocycle, the position of each ncAA is defined by the genetic sequence, and the identity of the ncAA at each position is defined by the reassignment scheme.

### Example 2

### Genetically Encoding Aliphatic Hydroxy Acids with Orthogonal PyIRS/tRNA Pairs

Next, we explored reassigning codons to hydroxy acids with a view towards the encoded cellular synthesis of depsipeptide macrocycles. The Mm and Methanosarcina barkeri (Mb) pyrrolysyl-tRNA synthetases and their cognate tRNAs have been used to incorporate four lysine hydroxy acid derivatives—with limited efficiency at single sites in proteins17-19. We set out to i) reassign the TCG sense codon in Syn61Δ3(ev5) to hydroxy acids and to ii) increase the chemical diversity of distinct hydroxy acids (FIG. 6a) that can be incorporated—via cellular translation—into protein and peptide sequences.

Using a liquid chromatography-mass spectrometry (LC-MS) assay, we demonstrated that BocK-OH (6), a representative aliphatic hydroxy acid, is taken up by E. coli when added to the growth media (FIG. 6b); we observed substantial conversion of BocK-OH to the corresponding amino acid. We expressed sfGFP-3-TCG (a superfolder GFP gene containing a TCG codon at position 3 and no other TCG codons within the gene) in Syn61Δ3(ev5) cells containing MmPylRS/MmtRNAPylCGA supplemented with 2 mM BocK-OH; ESI-MS, under buffer conditions which induce ester bond cleavage, demonstrated selective BocK-OH incorporation (FIG. 6c). Thus, despite substantial intracellular conversion of BocK-OH to the corresponding amino acid, we observe the selective incorporation of BocK-OH with the MmPylRS/MmtRNAPylCGA pair. Notably, we see very little, if any, intracellular conversion of BocK to BocK-OH (FIG. 6b). We conclude that when BocK-OH is added to cells there is substantial metabolic conversion to BocK, and the MmPylRS/MmtRNAPyl pair selectively incorporates BocK-OH into proteins in preference to BocK. However, when BocK is added to cells it is not substantively converted to BocK-OH and the MmPylRS/MmtRNAPyl pair incorporates BocK. Both these results are consistent with prior observations demonstrating that the MmPylRS/MmtRNAPylCUA pair can incorporate amino acids or hydroxy acids when these compounds are provided as substrates17, 18. However, our results demonstrate that these prior observations are the result of: i) asymmetry in the cell's metabolism—the hydroxy acid is converted to the amino acid, but the amino acid is not substantively converted to the hydroxy acid—and ii) the selectivity of MmPylRS for the hydroxy acid.

Next, we investigated the incorporation of seven additional hydroxy acids (7-13) with aliphatic side chains (FIG. 6a); 6, 7, and 12 have previously been incorporated with PyIRS variants17, 19, 20. We investigated the incorporation of each hydroxy acid (6-13) with the MmPylRS/MmtRNAPylCGA pair, the 1R26PylRS/A/vRNAΔNPyl(8)CUA pair2l, and several active site variants of each pair. We identified aaRS/tRNA pairs that led to robust—hydroxy acid dependent—increases in sfGFP-3-TCG or sfGFP-3-TAG expression (FIG. 6d). To confirm the selective incorporation of each hydroxy acid into sfGFP with a cognate aaRS/tRNA pair, we purified the protein and measured its mass by ESI-MS under buffer conditions which induce ester bond cleavage. We observed selective hydroxy acid incorporation in all cases (FIG. 6e).

Notably, when cells containing PyIRS/tRNACUA pairs and sfGFP-3-TAG were supplemented with both amino acid and hydroxy acid (for 6, 7, 8, 12 and 13 and their respective amino acid analogues), we observed only hydroxy acid incorporation into sfGFP (FIG. 7).

These results are consistent with PyIRS systems being selective for hydroxy acids, with aliphatic side chains, over the corresponding amino acids.

### Example 3

### Genetically Encoding Hydroxy Acids with Aromatic Sidechains

Next, we aimed to incorporate hydroxy acids with aromatic sidechains (FIG. 8a). We tested the AfTyrRS(plF)/AftRNATyr(A01)CUA pair, which can incorporate p-lodo-L-phenylalanine, for its ability to incorporate the corresponding hydroxy analogue (15, plF-OH), and the Methanocaldococcus jannaschii (Mj)TyrRS(Nap)/MjtARNATyrCUA pair, which can incorporate L-3-(2-Naphthyl)alanine)24, for its ability to incorporate the corresponding hydroxy analogue (16, NapA-OH). ESI-MS analysis of sfGFP purified from cells containing each aaRS/tRNA pair, the corresponding hydroxy acid, and sfGFP-3-TAG revealed incorporation of the corresponding amino acid into sfGFP (FIG. 9). We demonstrated that these aromatic hydroxy acids are converted to the corresponding amino acids in cells (FIG. 9). Previous work reported that deletion of two transaminases aspC and tyrB in E. coli enables the biosynthetic accumulation of p-hydroxy-L-phenyllactic acid, at sufficient levels to direct its incorporation using an engineered MjTyrRS/MjtRNATyrCUA pair⁵⁷.

Disrupting these two transaminases slightly increased the intracellular levels of added 15 and 16, but did not stop their conversion to the corresponding amino acids. Moreover, sfGFP purified from ΔaspC/ΔtyrB double knockout cells—containing the AfTyrRS(plF)/AftRNATyr(A01)CUA or the MjTyrRS(Nap)/MjtRNATyrCUA pair, 15 or 16, and sfGFP-3-TAG—exclusively incorporated the corresponding amino acid analogues (FIG. 9).

We hypothesized that TyrRS derivatives are selective for amino acids with aromatic sidechains over the corresponding hydroxy acids; this hypothesis is consistent with structures of AfTyrRS in complex with tyrosine, which shows that the amino group of tyrosine is specifically coordinated by Y158, Q162, and Q180 residues in the enzyme⁵⁸. Because the MmPylRS/MmtRNAPyl pair is selective for aliphatic hydroxy acids over the corresponding amino acids, and aromatic amino acids have been incorporated using engineered mutants of this pair⁵⁹, we hypothesized that it might be possible to engineer the MmPylRS/MmtRNAPyl pair for the selective cellular incorporation of hydroxy acids with aromatic sidechains, despite the metabolic interconversion of the hydroxy acids to amino acids (FIG. 8b). We note that previously reported mutants of PyIRS were permissive to incorporating aromatic hydroxy acids but also incorporated phenylalanine⁴³.

We constructed a MmPylRS library by randomizing the codons at five positions in the active site (M300, L301, A302, M344, and N346) with degenerate codons. These residues are proximal to the substrate's α-amino group, and are involved in preventing the binding of phenylalanine to MmPylRS28. We subjected the library to one round of positive selection in the presence of 14, the hydroxy acid analogue of phenylalanine; the selection was based on the ability to read through an amber stop codon in a chloramphenicol acetyltransferase gene (CATD111-TAG) and confer resistance to chloramphenicol29. We screened the resulting MmPylRS/MmtRNAPylCUA derivatives to identify clones that generated GFP fluorescence from sfGFP-3-TAG in the presence of 14, but not in the absence of 14; this yielded six mutants, MmPylRS(PheOH_1) to MmPylRS(PheOH_6), which directed the selective incorporation of 14 (FIG. 8c, FIG. 11).

Next, to encode further alpha hydroxy acids with aromatic sidechains (15 and 16), we created saturation mutagenesis libraries based on MmPylRS(PheOH_1), MmPylRS(PheOH_3), and MmPylRS(PheOH_6). These libraries targeted three residues (C348, V401, W417) which delimit the pocket in the enzyme that binds to the side chain of its substrates. We subjected the pooled libraries to positive selection in the presence of 15 or 16 using the CAT-D111-TAG selection marker. We further analysed surviving clones from the selection in the presence of 15, using an sfGFP-3-TAG reporter, for their activity in the presence and absence of 15 and 16; this allowed us to identify one variant MmPylRS(ArOH), derived from MmPylRS(PheOH_6), with the desired properties. The MmPylRS(ArOH)/MmtRNAPylCUA pair selectively incorporated 15 or 16 to produce sfGFP from sfGFP-3-TAG in E. coli cells and exhibited little activity towards phenylalanine or the amino acid analogues of 15 and 16 (FIG. 8c, d, FIG. 10a, FIG. 11). We demonstrated that the active site of ArOH-RS (as well as PheOH-RS1 and PheOH-RS6) effectively discriminated against hydroxy acids with aliphatic sidechains (compounds 6-13) (FIG. 10a).

### Example 4

### Discovering Mutually Orthogonal Pairs for Encoding Non-Canonical Amino Acids and Hydroxy Acids

Class +N pyrrolysyl pairs (including MmPylRS/MmtRNAPyl), ΔN Class A pyrrolysyl pairs (including 1R26PyIRS/A/vRNAΔNPyl(8)), and the AfTyrRS/AftRNATyr or MjTyrRS/MjtARNATyr are mutually orthogonal in their aminoacylation specificity¹⁶³⁰. In order to use combinations of these pairs to encode distinct non-canonical monomers they must be engineered to specifically decode distinct codons and specifically recognize distinct non-canonical monomers. The pyrrolysyl tRNAs can be engineered to recognize many different codons⁶⁰ and the tyrosyl tRNAs can be directed to the amber codon⁶¹, and therefore all combinations of distinct pairs can be directed to decode TAG and TCG codons. Thus, the outstanding challenge was to define mutually orthogonal non-canonical monomer specificity for Class+N pyrrolysyl pairs, ΔN Class A pyrrolysyl pairs and the tyrosyl pairs.

We expressed each aaRS/tRNA pair, and several active site variants, with 5 ncAAs and 11 hydroxy acids, and we measured production of sfGFP from sfGFP-3-XXX (where XXX is TAG for ΔN Class A pyrrolysyl-derived pairs and tyrosyl-derived pairs, and TCG for Class+N pyrrolysyl-derived pairs) in Syn61Δ3(ev5) (FIG. 10a). From the matrix of 116 combinations of aaRS/tRNA pairs and monomers we directly identified 77 pairs of mutually orthogonal active sites (and pairs of cognate monomers) in synthetases that are mutually orthogonal in their tRNA specificity (FIG. 10b-e, FIG. 12). We also identified 49 unique reassignment schemes (Table 1). We focussed on further characterizing a subset of these aaRS/tRNA pair combinations as the basis of additional reassignment schemes for encoding a ncAA and a hydroxy acid or for encoding two hydroxy acids (FIG. 10b-f). Within most of the pairs of aaRS/tRNA pairs we characterized in detail, each aaRS directs the incorporation of its cognate monomer to synthesize full length sfGFP, but minimally directs the incorporation of the monomer that is efficiently incorporated by the other aaRS/tRNA pair 16 (FIG. 10b-e). In addition, when cells containing both aaRS/tRNA pairs were provided with both non-canonical monomers we observed selective incorporation of the cognate monomer into sfGFP by each aaRS/tRNA pair, as judged by mass spectrometry (FIG. 10b-e). These observations indicated that these Class+N pyrrolysyl pairs, ΔN, Class A pyrrolysyl pairs and tyrosyl pairs have active sites with mutually orthogonal specificity for specific combinations of ncAAs and hydroxy acids, as required to incorporate a ncAA and a hydroxy acid into a single genetically encoded polymer within the cell.

We noted that the MmPylRS/MmtRNAPylCGA pair efficiently incorporated 1 (AllocK) but not 4 (CbzK), and that the same pair incorporated both 7 (AllocK-OH) and 12 (CbzK-OH). Similarly, we observed that the 1R26PyIRS(CbzK)/AvtRNAΔNPyl(8)CUA pair efficiently incorporated 4 (CbzK) but not 1 (AllocK), and that the same pair incorporated both 12 (CbzKOH) and 7 (AllocK-OH). We hypothesized that the intrinsic preference of MmPylRS for the AllocK side chain in 1 over the CbzK sidechain in 4 might be preserved in the recognition of the corresponding hydroxy acids. Similarly, we hypothesized that the intrinsic preference of 1R26PyIRS(CbzK) for the CbzK side chain in 4 over the AllocK side chain in 1 might be preserved in the recognition of the corresponding hydroxy acids. Indeed, when cells containing both the MmPylRS/MmtRNAPylCGA pair and the 1R26PyIRS(CbzK)/A/vRNAΔNPyl(8)CUA pair were provided with both 7 and 12 we observed selective incorporation of 7 in response to the TCG codon in sfGFP-3-TCG and selective incorporation of 12 in response to the TAG codon in sfGFP-3-TAG (FIG. 4f). These observations indicated that these Class+N pyrrolysyl pairs, and ΔN, Class A pyrrolysyl pairs have active sites with mutually orthogonal hydroxy acid specificity, as required to incorporate two distinct hydroxy acids into a single genetically encoded polymer within the cell.

### Example 5

### Combinatorially Encoding Non-Canonical Hydroxy Acids in Macrocyclic Depsipeptides

To synthesize macrocyclic depsipeptides we combined the macrocycle-encoding gene fusions (FIG. 1) with reassignment schemes 4-8 (FIG. 12); these reassignment schemes were defined based on the aaRS/tRNA specificity we have defined (FIG. 10). In r.s. 4, monomer A is 7 (AllocK-OH) and is incorporated using the MmPylRS/MmtRNAPylCGA pair, and monomer B is 3 (plF) and is incorporated using the AfTyrRS(plF)/AftRNATyr(A01)CUA pair. In r.s. 5, monomer A is 7 (AllocK-OH), and is incorporated using the MmPylRS/MmtRNAPylCGA pair, and monomer B is 12 (CbzK-OH), and is incorporated using the 1R26PyIRS(CbzK)/A/vRNAΔNPyl(8)CUA pair. In r.s. 6, monomer A is 8 (AlkynK-OH), and is incorporated using the MmPylRS/MmtRNAPylCGA pair and monomer B is 5 (pAzF) and is incorporated using the AfTyrRS(pAzF)/AftRNATyr(A01)CUA pair. In r.s. 7, monomer A is 14 (FOH), and is incorporated using the MmPylRS(PheOH_6)/MmtRNAPylCGA pair and monomer B is 4 (CbzK) and is incorporated using the 1R26PyIRS/A/vRNAΔNPyl(8)CUA pair. In r.s. 8, monomer A is 16 (NapA)-OH, and is incorporated using the MmPylRS(ArOH)/MmtRNAPylCGA pair and monomer B is 1 (AllocK) and is incorporated using the 1R26PyIRS/A/vRNAΔNPyl(8)CUA pair. These new reassignment schemes introduce non-natural side chains and also introduce one or two ester bonds into the backbone of the macrocycle (FIG. 12a, b).

We produced, isolated and confirmed the exact mass for 12 depsipeptides (FIG. 14b, FIG. 13) and ten of these isolated depsipeptides were macrocyclic; we note that for 3 of these 10 depsipeptides we observed that a fraction of the molecules was not cyclized (FIG. 15). Two depsipeptides were isolated as hydrolysed linear products in which the ester bond was cleaved (FIG. 12b), such hydrolysis is likely to be sequence dependent. For depsipeptides made with r.s. 7 and r.s. 8. the yields of full-length SUMO-depsipeptide-GyrA-CDB were low (0-0.4 mg per L of culture); this may explain why 83% of the depsipeptides we aimed to make using these recoding schemes could either not be identified by ESI MS (FIG. 12b) or had very low ESI-MS peaks (YMBA r.s.8, SanA1AB r.s. 8, FIG. 13). The yields of full-length SUMO-depsipeptide-GyrA-CDB for depsipeptides made with r.s. 4-6 were higher (0.8-6.5 mg per L of culture, FIG. 3), and the Ulp1-mediated cleavage led to disappearance of SUMO-peptide-GyrA-CBD and the appearance of GyrA-CBD on SDS-PAGE (FIG. 3), consistent with depsipeptide excision. The variation in the intensity of the MS signal (FIG. 13) (or absence of MS signal, (FIG. 14b)) for extracted depsipeptides made with these recoding schemes is likely to result from differences in the extracted yields of the depsipeptides, or from differences in the ionization efficiencies of the extracted depsipeptides in mass spectrometry.

We further analysed the depsipeptide produced from SanA2BA with r.s. 6 (FIG. 14c) by tandem mass spectrometric analysis (MS/MS). The cyclic molecule can theoretically fragment into 5 linear sequences (FIG. 14d), which can further fragment. We identified at least 3 fragments for each of the five linear sequences for this molecule, further confirming that the isolated depsipeptide is cyclic and has the expected sequence of monomers (FIG. 14d, e).

### REFERENCES

3. J. W. Chin, Expanding and reprogramming the genetic code. Nature 550, 53-60 (2017).
4. D. de la Torre, J. W. Chin, Reprogramming the genetic code. Nature Reviews Genetics 22, 169-184 (2020).
5. T. Passioura, H. Suga, Reprogramming the genetic code in vitro. Trends Biochem Sci 39, 400-408 (2014).
6. A. C. Forster et al., Programming peptidomimetic syntheses by translating genetic codes designed de novo. Proc Natl Acad Sci USA 100, 6353-6357 (2003).
10. D. D. Young, P. G. Schultz, Playing with the Molecules of Life. ACS Chemical Biology 13, 854-870 (2018).
11. C. C. Liu, P. G. Schultz, Adding New Chemistries to the Genetic Code. Annual Review of Biochemistry 79, 413-444 (2010).
12. Y. Zhang et al., A semi-synthetic organism that stores and retrieves increased genetic information. Nature 551, 644-647 (2017).
13. E. C. Fischer et al., New codons for efficient production of unnatural proteins in a semisynthetic organism. Nature Chemical Biology 16, 570-576 (2020).
14. H. Neumann, K. Wang, L. Davis, M. Garcia-Alai, J. W. Chin, Encoding multiple unnatural amino acids via evolution of a quadruplet-decoding ribosome. Nature 464, 441-444 (2010).
15. K. Wang et al., Optimized orthogonal translation of unnatural amino acids enables spontaneous protein double-labelling and FRET. Nature Chemistry 6, 393-403 (2014).
16. D. L. Dunkelmann, J. C. W. Willis, A. T. Beattie, J. W. Chin, Engineered triply orthogonal pyrrolysyl-tRNA synthetase/tRNA pairs enable the genetic encoding of three distinct non-canonical amino acids. Nature Chemistry 12, 535-544 (2020).
17. J. S. Italia et al., Mutually Orthogonal Nonsense-Suppression Systems and Conjugation Chemistries for Precise Protein Labeling at up to Three Distinct Sites. Journal of the American Chemical Society 141, 6204-6212 (2019).
18. J. Fredens et al., Total synthesis of Escherichia coli with a recoded genome. Nature 569, 514-518 (2019).
30. D. Cervettini et al., Rapid discovery and evolution of orthogonal aminoacyl-tRNA synthetase—tRNA pairs. Nature Biotechnology 38, 989-999 (2020).
42. Zambaldo, C., Luo, X., Mehta, A. P. & Schultz, P. G. Recombinant Macrocyclic Lanthipeptides Incorporating Non-Canonical Amino Acids. J Am Chem Soc 139, 11646-11649, doi:10.1021/jacs.7b04159 (2017).
43. Bindman, N. A., Bobeica, S. C., Liu, W. R. & van der Donk, W. A. Facile Removal of Leader Peptides from Lanthipeptides by Incorporation of a Hydroxy Acid. J Am Chem Soc 137, 6975-6978, doi:10.1021/jacs.5b04681 (2015).
44. Montalbán-Lopez, M. et al. New developments in RiPP discovery, enzymology and engineering. Natural Product Reports 38, 130-239, doi:10.1039/DONP00027B (2021). 23
45. Süssmuth, R. D. & Mainz, A. Nonribosomal Peptide Synthesis-Principles and Prospects. Angew Chem Int Ed Engl 56, 3770-3821, doi:10.1002/anie.201609079 (2017).
46. Bozhuyuk, K. A. J. et al. Modification and de novo design of non-ribosomal peptide synthetases using specific assembly points within condensation domains. Nature Chemistry 11, 653-661, doi:10.1038/s41557-019-0276-z (2019).
47. Niquille, D. L. et al. Nonribosomal biosynthesis of backbone-modified peptides. Nature Chemistry 10, 282-287, doi:10.1038/nchem.2891 (2018).
48. Maini, R., Umemoto, S. & Suga, H. Ribosome-mediated synthesis of natural product like peptides via cell-free translation. Curr Opin Chem Biol 34, 44-52, doi:10.1016/j.cbpa.2016.06.006 (2016).
49. Ohta, A., Murakami, H., Higashimura, E. & Suga, H. Synthesis of polyester by means of genetic code reprogramming. Chem Biol 14, 1315-1322, doi:10.1016/j.chembiol.2007.10.015 (2007).
50. Ohta, A., Murakami, H. & Suga, H. Polymerization of alpha-hydroxy acids by ribosomes. Chembiochem 9, 2773-2778, doi:10.1002/cbic.200800439 (2008).
51. Scott, C. P., Abel-Santos, E., Wall, M., Wahnon, D. C. & Benkovic, S. J. Production of cyclic peptides and proteins in vivo. Proc Natl Acad Sci USA 96, 13638-13643, doi:10.1073/pnas.96.24.13638 (1999).
52. Robertson, W. E. et al. Sense codon reassignment enables viral resistance and encoded polymer synthesis. Science 372, 1057-1062, doi:10.1126/science.abg3029 (2021).
53. Huguenin-Dezot, N. et al. Trapping biosynthetic acyl-enzyme intermediates with encoded 2,3-diaminopropionic acid. Nature 565, 112-117, doi:10.1038/s41586-018-0781-z (2019).
54. Li, Y.-M. et al. Ligation of Expressed Protein α-Hydrazides via Genetic Incorporation of an α-Hydroxy Acid. ACS Chemical Biology 7, 1015-1022, doi:10.1021/cb300020s (2012).
55. Belofsky, G. N., Jensen, P. R. & Fenical, W. Sansalvamide: A new cytotoxic cyclic depsipeptide produced by a marine fungus of the genus Fusarium. Tetrahedron Letters 40, 2913-2916, doi:https://doi.org/10.1016/S0040-4039(99)00393-7 (1999).
56. Peng, Q. & Shen, J. YM-254890 is a General Inhibitor of G Proteins. The FASEB Journal 33, 503.507-503.507, doi:https://doi.org/10.1096/fasebj.2019.33.1_supplement.503.7 (2019).
57. Guo, J., Wang, J., Anderson, J. C. & Schultz, P. G. Addition of an alpha-hydroxy acid to the genetic code of bacteria. Angew Chem Int Ed Engl 47, 722-725, doi:10.1002/anie.200704074 (2008).
58. Kuratani, M. et al. Crystal structures of tyrosyl-tRNA synthetases from Archaea. J Mol Biol 355, 395-408, doi:10.1016/j.jmb.2005.10.073 (2006).
59. Wang, Y. S., Fang, X., Wallace, A. L., Wu, B. & Liu, W. R. A rationally designed pyrrolysyl-tRNA synthetase mutant with a broad substrate spectrum. J Am Chem Soc 134, 2950-2953, doi:10.1021/ja211972x (2012).
60. Ambrogelly, A. et al. Pyrrolysine is not hardwired for cotranslational insertion at UAG codons. Proc Natl Acad Sci USA 104, 3141-3146, doi:10.1073/pnas.0611634104 (2007).
61. Wang, L., Magliery, T. J., Liu, D. R. & Schultz, P. G. A new functional suppressor tRNA/aminoacyl-tRNA synthetase pair for the in vivo incorporation of unnatural amino acids into proteins. J Am Chem Soc 122, 5010-5011, doi:DOI 10.1021/ja000595y (2000).

1. A prokaryotic cell, wherein:
a. at least one sense codon is reassigned to a first orthogonal tRNA synthase/tRNA pair;
b. at least one sense or nonsense codon is reassigned to a second orthogonal tRNA synthase/tRNA pair;
c. the first and second orthogonal tRNA synthase/tRNA pair are further mutually orthogonal; and
d. both the first and second orthogonal tRNA synthase/tRNA pairs preferentially recognise non-canonical, non-alpha-amino acids.

2. The prokaryotic cell according to claim 1, wherein a second sense codon is reassigned to a third orthogonal tRNA synthase/tRNA pair which preferentially recognises a non-alpha amino acid, said pair being further orthogonal to the first and second tRNA synthase/tRNA pairs.

3. The prokaryotic cell according to claim 1, wherein the non-alpha amino acids are:
a. alpha-hydroxy acids,
b. alpha-hydroxy acids with an aromatic side-chain; or
c. alpha-hydroxy acids with an aliphatic side-chain.

4. The prokaryotic cell according to claim 1, wherein the sense codon or codons are absent from essential genes of the prokaryotic cell, and the genome of the prokaryotic cell comprises 5, 4, 3, 2, 1 or no occurrences of any one of said sense codons.

5. The prokaryotic cell according to claim 1, wherein the endogenous tRNA(s) cognate for said sense codon(s) are not expressed by the prokaryotic cell.

6. The prokaryotic cell according to claim 1, wherein the sense codons are selected from the group consisting of TCA and TCG.

7. The prokaryotic cell according to claim 1, wherein the nonsense codon is TAG and the prokaryotic cell does not encode endogenous release factor 1 (RF-1).

8. A prokaryotic cell according claim 3, wherein the non-alpha-amino acid is:
a. an alpha-hydroxy acids aromatic side-chain, selected from F-OH, plF-OH and NapA-OH, or
b. an alpha-hydroxy acids with an aliphatic side-chain, selected from BocK-OH, PenK-OH, AllocK-OH, NorK-OH, AlkynK-OH, CbzK-OH, ButK-OH and AcK-OH.

9. The prokaryotic cell according to claim 1, which expresses a heterologous gene encoding a polymer which comprises two or more non-canonical non-aminoacids.

10. The prokaryotic cell according to claim 9, wherein the polymer also comprises one or more canonical or non-canonical amino acids.

11. The prokaryotic cell according to claim 9, wherein the non-amino acids are hydroxy acids.

12. The prokaryotic cell according to claim 10, wherein the polymer backbone comprises one or more ester bonds.

13. The prokaryotic cell according to claim 12, wherein the polymer backbone comprises two or more ester bonds.

14. The prokaryotic cell according to claim 9, wherein the polymer is cyclic.

15. The prokaryotic cell according to claim 9, wherein the polymer is a depsipeptide (macrocyclic depsipeptide).

16. A method for producing a modified prokaryotic cell, wherein the method comprises:
(i) modifying a prokaryotic cell to express a first orthogonal tRNA synthetase—tRNA pair suitable for decoding a first type of sense codon, and a second orthogonal tRNA synthetase—tRNA pair suitable for decoding a second type of sense codon wherein the prokaryotic cell comprises a genome wherein the first and second types of sense codon have been recoded such that a first and second endogenous tRNA are dispensable;
(ii) incubating the prokaryotic cell in the presence of first and second non-alpha-amino acids which are substrates for the first and second orthogonal tRNA synthetases; and
(iii) modifying the endogenous genes encoding the first and second endogenous tRNAs such that the first and second endogenous tRNAs are not expressed.

17. The method of claim 16, wherein the method further comprises:
(a) modifying the prokaryotic cell to express a third orthogonal aminoacyl-tRNA synthetase—tRNA pair suitable for decoding a third type of sense codon, wherein
the prokaryotic cell comprises a genome wherein the third type of sense codon has been recoded such that a third endogenous tRNA is dispensable;
(b) incubating the prokaryotic cell in the presence of a third non-alpha-amino acid which is a substrate for the third orthogonal aminoacyl-tRNA synthetase; and
(c) modifying the endogenous gene encoding the third endogenous tRNA such that the third endogenous tRNA is not expressed.

18. The method of claim 16, wherein the first type of sense codon is TCA and the first endogenous tRNA is tRNA^{Ser}_{UGA}.

19. The method of claim 18, wherein the TCA codon has been replaced with AGT.

20. The method of claim 16, wherein the first orthogonal tRNA synthetase is MmPylRS or a variant with altered selectivity to a non-alpha amino acid, and the first orthogonal tRNA is MmtRNA^{PylY}_{YYY} or MmtRNA^{Pyl}_{UGA}.

21. The method of claim 16, wherein the second type of sense codon is TCG and the second endogenous tRNA is tRNA^{Ser}_{CGA}.

22. The method of claim 21, wherein the TCG codon is replaced with AGC.

23. The method of claim 16, wherein the second orthogonal tRNA synthetase is methoaldococcus jannaschii TyrRS or a variant with altered selectivity to a non-canonical amino acid, and the second orthogonal tRNA is MjtRNA^{Tyr}_{CUA}.

24. The method of claim 16, further comprising modifying the prokaryotic cell to express an orthogonal tRNA synthetase—tRNA pair suitable for decoding a first type of stop codon, wherein a first type of stop codon has been recoded within the genome such that a first endogenous release factor is dispensable.

25. The method of claim 24, wherein the essential genes of the genome do not contain occurrences of the first type of stop codon or the genome comprises 5, 4, 3, 2, 1, or no occurrences of the first type of stop codon.

26. The method of claim 24, wherein the first type of stop codon is TAG, and wherein the cognate release factor for the first type of stop codon is RF-1.

27. The method of claim 26, wherein the TAG codon is replaced with TAA.

28. The method of claim 17, wherein:
a. the first and second aaRS/tRNA pairs are selected such that their monomer incorporation preferences are specific for different alpha-hydroxy acid monomers: or
b. the third orthogonal aminoacyl-tRNA synthetase—tRNA pair is AfTyrRS or a variant with altered selectivity to a non-alpha-amino acid, and Af-tRNA^{Tyr(A01)}_{YYY}.

29. The method of claim 16, wherein the prokaryotic cell is a bacterial cell or an Escherichia coli cell.

30. A nucleic acid encoding a macrocyclic depsipeptide, said nucleic acid encoding:
a. an N-terminal small ubiquitin-like modifier (SUMO) element followed by a cysteine;
b. the sequence of a depsipeptide macrocycle, said sequence comprising two or more codons which are recognised by two or more orthogonal RNA synthase/tRNA pairs, said pairs preferentially recognising an alpha-hydroxy acid, said codons being selected from TCA, TCG and TAG; and
c. A GyrA intein-CBD fusion element C-terminal to the sequence in (b).

31. A method for producing a depsipeptide macrocycle, comprising expressing a nucleic acid according to claim 30 in a prokaryotic cell according to claim 1 such that alpha-hydroxy monomers are incorporated into the polymer encoded by the nucleic acid.

32-33. (canceled)

34. A modified pyrrolysyl or tyrolysyl amino acid tRNA synthetase (aaRS) and paired tRNA which preferentially bind to alpha-hydroxy acids, optionally with an aromatic side-chain.

35. A method for selecting a pyrrolysyl/tyrolysyl aaRS which preferentially incorporates alpha-hydroxy acids over their amino acid equivalent, comprising the steps of: constructing a library of aaRS genes, using degenerate codons in the active site of the enzyme proximal to the location of the alpha-amino group of a bound amino acid; screening the library in the presence of the target alpha-hydroxy acid; identifying clones which incorporate alpha-hydroxy acids, and subjecting said clones to negative screening against alpha-amino acids to select against incorporation of amino acids.


SEQUENCE LISTING (106 sequences)

SEQ ID NO: 1 - DNA, 95 bp, synthetic construct
  source 1..95: mol_type = other DNA
tcaggcaatgttgcgtttgtcatcagtctcagcccgcttttcagcgggcttcattgtttttaatgcttattcctttgccctcggacgagtgctgg

SEQ ID NO: 2 - DNA, 95 bp, synthetic construct
  source 1..95: mol_type = other DNA
agtaatagttggaattttgtaaatctcccgttaccctgatagcggacttcccttctgtaaccatagcacgtgttgacaattaatcatcggcatag

SEQ ID NO: 3 - DNA, 27 bp, synthetic construct
  source 1..27: mol_type = other DNA
tattgcaggggacgcgtaggcctgata

SEQ ID NO: 4 - DNA, 47 bp, synthetic construct
  source 1..47: mol_type = other DNA
cccttctgtaaccatgcattaaaaacaatgaagcccgctgaaaagcg

SEQ ID NO: 5 - DNA, 49 bp, synthetic construct
  source 1..49: mol_type = other DNA
gcttcattgtttttaatgcatggttacagaagggaagtccgctatcagg

SEQ ID NO: 6 - DNA, 27 bp, synthetic construct
  source 1..27: mol_type = other DNA
ctcacgtcttgcaaaaacagcctgcgt

SEQ ID NO: 7 - DNA, 27 bp, synthetic construct
  source 1..27: mol_type = other DNA
tattgcaggggacgcgtaggcctgata

SEQ ID NO: 8 - DNA, 27 bp, synthetic construct
  source 1..27: mol_type = other DNA
ctcacgtcttgcaaaaacagcctgcgt

SEQ ID NO: 9 - DNA, 25 bp, synthetic construct
  source 1..25: mol_type = other DNA
cctggatttctggcaaagtgcgctt

SEQ ID NO: 10 - DNA, 34 bp, synthetic construct
  source 1..34: mol_type = other DNA
atcgtttaccagttctaatagcacacctctttgt

SEQ ID NO: 11 - DNA, 95 bp, synthetic construct
  source 1..95: mol_type = other DNA
tgaacatccactcgatcttcgccttcttccggtttattgtgttttaaccacctgcccgtaaacctgttattcctttgccctcggacgagtgctgg

SEQ ID NO: 12 - DNA, 95 bp, synthetic construct
  source 1..95: mol_type = other DNA
gagtgaaagcgacgacagtttaatttcactgcaggctgggtagctccagcctgctttcctgcaagcacgtgttgacaattaatcatcggcatagt

SEQ ID NO: 13 - DNA, 29 bp, synthetic construct
  source 1..29: mol_type = other DNA
gcctctggcggatgtacgtttgtcatgag

SEQ ID NO: 14 - DNA, 43 bp, synthetic construct
  source 1..43: mol_type = other DNA
gcctgctttcctgcacaggtttacgggcaggtggttaaaacac

SEQ ID NO: 15 - DNA, 40 bp, synthetic construct
  source 1..40: mol_type = other DNA
gcccgtaaacctgtgcaggaaagcaggctggagctaccca

SEQ ID NO: 16 - DNA, 34 bp, synthetic construct
  source 1..34: mol_type = other DNA
tccagataattgggataagcttaacaataaaaga

SEQ ID NO: 17 - DNA, 29 bp, synthetic construct
  source 1..29: mol_type = other DNA
gcctctggcggatgtacgtttgtcatgag

SEQ ID NO: 18 - DNA, 34 bp, synthetic construct; identical to SEQ ID NO: 16
  source 1..34: mol_type = other DNA

SEQ ID NO: 19 - DNA, 25 bp, synthetic construct
  source 1..25: mol_type = other DNA
cggatgcggcgtgaatgccttatcc

SEQ ID NO: 20 - DNA, 34 bp, synthetic construct
  source 1..34: mol_type = other DNA
ggcaaagcatattcttgaggatatagaaatattc

SEQ ID NO: 21 - DNA, 42 bp, synthetic construct
  source 1..42: mol_type = other DNA
gtggttgcctgtagctgtcgtgcatcacgggagatgcactag

SEQ ID NO: 22 - DNA, 39 bp, synthetic construct
  source 1..39: mol_type = other DNA
tgcacgacagctacaggcaaccaccaatctgttctctgt

SEQ ID NO: 23 - DNA, 42 bp, synthetic construct
  source 1..42: mol_type = other DNA
gtggttgctagctggtttcgtgcatcacgggagatgcactag

SEQ ID NO: 24 - DNA, 39 bp, synthetic construct
  source 1..39: mol_type = other DNA
tgcacgaaaccagctagcaaccaccaatctgttctctgt

SEQ ID NO: 25 - DNA, 34 bp, synthetic construct
  source 1..34: mol_type = other DNA
atgcacacctgcaaggcatcacgggagatgcact

SEQ ID NO: 26 - DNA, 38 bp, synthetic construct
  source 1..38: mol_type = other DNA
atgcacacctgcttaccaccaatctgttctctgtgagc

SEQ ID NO: 27 - DNA, 23 bp, synthetic construct
  source 1..23: mol_type = other DNA
ggtggttgcctatcgctgtagtg

SEQ ID NO: 28 - DNA, 23 bp, synthetic construct
  source 1..23: mol_type = other DNA
gatgcactacagcgataggcaac

SEQ ID NO: 29 - DNA, 26 bp, synthetic construct
  source 1..26: mol_type = other DNA
ggtggttgcctatcgctgtagctttg

SEQ ID NO: 30 - DNA, 26 bp, synthetic construct
  source 1..26: mol_type = other DNA
gatgcaaagctacagcgataggcaac

SEQ ID NO: 31 - DNA, 26 bp, synthetic construct
  source 1..26: mol_type = other DNA
ggtggttgcctatagctgtcgctttg

SEQ ID NO: 32 - DNA, 26 bp, synthetic construct
  source 1..26: mol_type = other DNA
gatgcaaagcgacagctataggcaac

SEQ ID NO: 33 - DNA, 26 bp, synthetic construct
  source 1..26: mol_type = other DNA
ggtggttgcctatcgctgtaggtttg

SEQ ID NO: 34 - DNA, 26 bp, synthetic construct
  source 1..26: mol_type = other DNA
gatgcaaacctacagcgataggcaac

SEQ ID NO: 35 - DNA, 26 bp, synthetic construct
  source 1..26: mol_type = other DNA
ggtggttgcctatagctgtcggtttg

SEQ ID NO: 36 - DNA, 26 bp, synthetic construct
  source 1..26: mol_type = other DNA
gatgcaaaccgacagctataggcaac

SEQ ID NO: 37 - DNA, 29 bp, synthetic construct
  source 1..29: mol_type = other DNA
ggtggttgctagacgacatcggcggcttg

SEQ ID NO: 38 - DNA, 29 bp, synthetic construct
  source 1..29: mol_type = other DNA
gatgcaagccgccgatgtcgtctagcaac

SEQ ID NO: 39 - DNA, 29 bp, synthetic construct
  source 1..29: mol_type = other DNA
ggtggttgctcgacgacataggcggcttg

SEQ ID NO: 40 - DNA, 29 bp, synthetic construct
  source 1..29: mol_type = other DNA
gatgcaagccgcctatgtcgtcgagcaac

SEQ ID NO: 41 - DNA, 58 bp, synthetic construct
  source 1..58: mol_type = other DNA
gatcggtctctgcgtccgsvtnnknnkccgaacctgtacaactacctgcgtaaactgg

SEQ ID NO: 42 - DNA, 39 bp, synthetic construct
  source 1..39: mol_type = other DNA
gatcggtctcgacgcaggcagaagtttttgtcaacacgg

SEQ ID NO: 43 - DNA, 58 bp, synthetic construct; identical to SEQ ID NO: 41
  source 1..58: mol_type = other DNA

SEQ ID NO: 44 - DNA, 39 bp, synthetic construct; identical to SEQ ID NO: 42
  source 1..39: mol_type = other DNA

SEQ ID NO: 45 - DNA, 58 bp, synthetic construct
  source 1..58: mol_type = other DNA
gatcggtctctgcgtccgvwgnnknnkccgaacctgtacaactacctgcgtaaactgg

SEQ ID NO: 46 - DNA, 39 bp, synthetic construct; identical to SEQ ID NO: 42
  source 1..39: mol_type = other DNA

SEQ ID NO: 47 - DNA, 58 bp, synthetic construct
  source 1..58: mol_type = other DNA
gatcggtctctgcgtccgtgsnnknnkccgaacctgtacaactacctgcgtaaactgg

SEQ ID NO: 48 - DNA, 39 bp, synthetic construct; identical to SEQ ID NO: 42
  source 1..39: mol_type = other DNA

SEQ ID NO: 49 - DNA, 45 bp, synthetic construct
  source 1..45: mol_type = other DNA
gatcggtctcctgccagatgggttctggttgcacccgtgaaaacc

SEQ ID NO: 50 - DNA, 54 bp, synthetic construct
  source 1..54: mol_type = other DNA
gatcggtctctggcagaaabscagabsggtgaattcttccaggtgttctttacc

SEQ ID NO: 51 - DNA, 45 bp, synthetic construct; identical to SEQ ID NO: 49
  source 1..45: mol_type = other DNA

SEQ ID NO: 52 - DNA, 54 bp, synthetic construct
  source 1..54: mol_type = other DNA
gatcggtctctggcagaaabscaggdwggtgaattcttccaggtgttctttacc

SEQ ID NO: 53 - DNA, 45 bp, synthetic construct; identical to SEQ ID NO: 49
  source 1..45: mol_type = other DNA

SEQ ID NO: 54 - DNA, 54 bp, synthetic construct
  source 1..54: mol_type = other DNA
gatcggtctctggcagaaabscagcwbggtgaattcttccaggtgttctttacc

SEQ ID NO: 55 - DNA, 45 bp, synthetic construct; identical to SEQ ID NO: 49
  source 1..45: mol_type = other DNA

SEQ ID NO: 56 - DNA, 54 bp, synthetic construct
  source 1..54: mol_type = other DNA
gatcggtctctggcagaaabscagscaggtgaattcttccaggtgttctttacc

SEQ ID NO: 57 - DNA, 45 bp, synthetic construct; identical to SEQ ID NO: 49
  source 1..45: mol_type = other DNA

SEQ ID NO: 58 - DNA, 54 bp, synthetic construct
  source 1..54: mol_type = other DNA
gatcggtctctggcagaagdwcagabsggtgaattcttccaggtgttctttacc

SEQ ID NO: 59 - DNA, 45 bp, synthetic construct; identical to SEQ ID NO: 49
  source 1..45: mol_type = other DNA

SEQ ID NO: 60 - DNA, 54 bp, synthetic construct
  source 1..54: mol_type = other DNA
gatcggtctctggcagaagdwcaggdwggtgaattcttccaggtgttctttacc

SEQ ID NO: 61 - DNA, 45 bp, synthetic construct; identical to SEQ ID NO: 49
  source 1..45: mol_type = other DNA

SEQ ID NO: 62 - DNA, 54 bp, synthetic construct
  source 1..54: mol_type = other DNA
gatcggtctctggcagaagdwcagcwbggtgaattcttccaggtgttctttacc

SEQ ID NO: 63 - DNA, 45 bp, synthetic construct; identical to SEQ ID NO: 49
  source 1..45: mol_type = other DNA

SEQ ID NO: 64 - DNA, 54 bp, synthetic construct
  source 1..54: mol_type = other DNA
gatcggtctctggcagaagdwcagscaggtgaattcttccaggtgttctttacc

SEQ ID NO: 65 - DNA, 45 bp, synthetic construct; identical to SEQ ID NO: 49
  source 1..45: mol_type = other DNA

SEQ ID NO: 66 - DNA, 54 bp, synthetic construct
  source 1..54: mol_type = other DNA
gatcggtctctggcagaacwbcagabsggtgaattcttccaggtgttctttacc

SEQ ID NO: 67 - DNA, 45 bp, synthetic construct; identical to SEQ ID NO: 49
  source 1..45: mol_type = other DNA

SEQ ID NO: 68 - DNA, 54 bp, synthetic construct
  source 1..54: mol_type = other DNA
gatcggtctctggcagaacwbcaggdwggtgaattcttccaggtgttctttacc

SEQ ID NO: 69 - DNA, 45 bp, synthetic construct; identical to SEQ ID NO: 49
  source 1..45: mol_type = other DNA

SEQ ID NO: 70 - DNA, 54 bp, synthetic construct
  source 1..54: mol_type = other DNA
gatcggtctctggcagaacwbcagcwbggtgaattcttccaggtgttctttacc

SEQ ID NO: 71 - DNA, 45 bp, synthetic construct; identical to SEQ ID NO: 49
  source 1..45: mol_type = other DNA

SEQ ID NO: 72 - DNA, 54 bp, synthetic construct
  source 1..54: mol_type = other DNA
gatcggtctctggcagaacwbcagscaggtgaattcttccaggtgttctttacc

SEQ ID NO: 73 - DNA, 45 bp, synthetic construct; identical to SEQ ID NO: 49
  source 1..45: mol_type = other DNA

SEQ ID NO: 74 - DNA, 54 bp, synthetic construct
  source 1..54: mol_type = other DNA
gatcggtctctggcagaascacagabsggtgaattcttccaggtgttctttacc

SEQ ID NO: 75 - DNA, 45 bp, synthetic construct; identical to SEQ ID NO: 49
  source 1..45: mol_type = other DNA

SEQ ID NO: 76 - DNA, 54 bp, synthetic construct
  source 1..54: mol_type = other DNA
gatcggtctctggcagaascacaggdwggtgaattcttccaggtgttctttacc

SEQ ID NO: 77 - DNA, 45 bp, synthetic construct; identical to SEQ ID NO: 49
  source 1..45: mol_type = other DNA

SEQ ID NO: 78 - DNA, 54 bp, synthetic construct
  source 1..54: mol_type = other DNA
gatcggtctctggcagaascacagcwbggtgaattcttccaggtgttctttacc

SEQ ID NO: 79 - DNA, 45 bp, synthetic construct; identical to SEQ ID NO: 49
  source 1..45: mol_type = other DNA

SEQ ID NO: 80 - DNA, 54 bp, synthetic construct
  source 1..54: mol_type = other DNA
gatcggtctctggcagaascacagscaggtgaattcttccaggtgttctttacc

SEQ ID NO: 81 - DNA, 49 bp, synthetic construct
  source 1..49: mol_type = other DNA
gatgggttctggttgcacccgtgaaaacctggaatctatcatcaccgac

SEQ ID NO: 82 - DNA, 50 bp, synthetic construct
  source 1..50: mol_type = other DNA
gggtgcaaccagaacccatctgmnngaaacccagcatggtgaattcttcc

SEQ ID NO: 83 - DNA, 49 bp, synthetic construct; identical to SEQ ID NO: 81
  source 1..49: mol_type = other DNA

SEQ ID NO: 84 - DNA, 50 bp, synthetic construct
  source 1..50: mol_type = other DNA
gggtgcaaccagaacccatctgmnngaaacccagcacggtgaattcttcc

SEQ ID NO: 85 - DNA, 49 bp, synthetic construct; identical to SEQ ID NO: 81
  source 1..49: mol_type = other DNA

SEQ ID NO: 86 - DNA, 50 bp, synthetic construct
  source 1..50: mol_type = other DNA
gggtgcaaccagaacccatctgmnngaaagccagcagggtgaattcttcc

SEQ ID NO: 87 - DNA, 58 bp, synthetic construct
  source 1..58: mol_type = other DNA
ccgctggaccgtgaatggggtatcgacaaaccgnnkatcggtgctggtttcggtctgg

SEQ ID NO: 88 - DNA, 62 bp, synthetic construct
  source 1..62: mol_type = other DNA
taccccattcacggtccagcgggatcggaccaacmnnagcagaagacagttccaggtcaccg

SEQ ID NO: 89 - RNA, 15 nt, synthetic construct
  source 1..15: mol_type = other RNA
  modified_base 1: mod_base = OTHER; note = A nucleotide sequence encoding a small ubiquitin-like modifier (SUMO) is fused to the 5' end of the nucleotide sequence
  modified_base 15: mod_base = OTHER; note = A nucleotide sequence encoding a GyrA intenin-chitin binding domain (intenin-CBD) fusion is fused to the 3' end of the nucleotide sequence
tgcctgtcgctgtag

SEQ ID NO: 90 - RNA, 15 nt, synthetic construct
  source 1..15: mol_type = other RNA
  modified_base 1: mod_base = OTHER; note = A nucleotide sequence encoding a small ubiquitin-like modifier (SUMO) is fused to the 5' end of the nucleotide sequence
  modified_base 15: mod_base = OTHER; note = A nucleotide sequence encoding an intenin-chitin binding domain (intenin-CBD) fusion is fused to the 3' end of the nucleotide sequence
tgctcgctggtttag

SEQ ID NO: 91 - RNA, 18 nt, synthetic construct
  source 1..18: mol_type = other RNA
  modified_base 1: mod_base = OTHER; note = A nucleotide sequence encoding a small ubiquitin-like modifier (SUMO) is fused to the 5' end of the nucleotide sequence
  modified_base 18: mod_base = OTHER; note = A nucleotide sequence encoding an intenin-chitin binding domain (intenin-CBD) fusion is fused to the 3' end of the nucleotide sequence
tgcctatcgctgtagctt

SEQ ID NO: 92 - RNA, 18 nt, synthetic construct
  source 1..18: mol_type = other RNA
  modified_base 1: mod_base = OTHER; note = A nucleotide sequence encoding a small ubiquitin-like modifier (SUMO) is fused to the 5' end of the nucleotide sequence
  modified_base 18: mod_base = OTHER; note = A nucleotide sequence encoding an intenin-chitin binding domain (intenin-CBD) fusion is fused to the 3' end of the nucleotide sequence
tgcctatcgctgtaggtg

SEQ ID NO: 93 - RNA, 21 nt, synthetic construct
  source 1..21: mol_type = other RNA
  modified_base 1: mod_base = OTHER; note = A nucleotide sequence encoding a small ubiquitin-like modifier (SUMO) is fused to the 5' end of the nucleotide sequence
  modified_base 21: mod_base = OTHER; note = A nucleotide sequence encoding an intenin-chitin binding domain (intenin-CBD) fusion is fused to the 3' end of the nucleotide sequence
tgctcgacgacataggcggct

SEQ ID NO: 94 - RNA, 15 nt, synthetic construct
  source 1..15: mol_type = other RNA
  modified_base 1: mod_base = OTHER; note = A nucleotide sequence encoding a small ubiquitin-like modifier (SUMO) is fused to the 5' end of the nucleotide sequence
  modified_base 15: mod_base = OTHER; note = A nucleotide sequence encoding an intenin-chitin binding domain (intenin-CBD) fusion is fused to the 3' end of the nucleotide sequence
tgcctgtagctgtcg

SEQ ID NO: 95 - RNA, 15 nt, synthetic construct
  source 1..15: mol_type = other RNA
  modified_base 1: mod_base = OTHER; note = A nucleotide sequence encoding a small ubiquitin-like modifier (SUMO) is fused to the 5' end of the nucleotide sequence
  modified_base 15: mod_base = OTHER; note = A nucleotide sequence encoding an intenin-chitin binding domain (intenin-CBD) fusion is fused to the 3' end of the nucleotide sequence
tgctagctggtttcg

SEQ ID NO: 96 - RNA, 18 nt, synthetic construct
  source 1..18: mol_type = other RNA
  modified_base 1: mod_base = OTHER; note = A nucleotide sequence encoding a small ubiquitin-like modifier (SUMO) is fused to the 5' end of the nucleotide sequence
  modified_base 18: mod_base = OTHER; note = A nucleotide sequence encoding an intenin-chitin binding domain (intenin-CBD) fusion is fused to the 3' end of the nucleotide sequence
tgcctatagctgtcgctt

SEQ ID NO: 97 - RNA, 18 nt, synthetic construct
  source 1..18: mol_type = other RNA
  modified_base 1: mod_base = OTHER; note = A nucleotide sequence encoding a small ubiquitin-like modifier (SUMO) is fused to the 5' end of the nucleotide sequence
  modified_base 18: mod_base = OTHER; note = A nucleotide sequence encoding an intenin-chitin binding domain (intenin-CBD) fusion is fused to the 3' end of the nucleotide sequence
tgcctatagctgtcggtg

SEQ ID NO: 98 - RNA, 21 nt, synthetic construct
  source 1..21: mol_type = other RNA
  modified_base 1: mod_base = OTHER; note = A nucleotide sequence encoding a small ubiquitin-like modifier (SUMO) is fused to the 5' end of the nucleotide sequence
  modified_base 21: mod_base = OTHER; note = A nucleotide sequence encoding an intenin-chitin binding domain (intenin-CBD) fusion is fused to the 3' end of the nucleotide sequence
tgctagacgacatcggcggct

SEQ ID NO: 99 - protein, 6 aa, synthetic construct
  MOD_RES 3: note = X is non-canonical monomer A
  MOD_RES 5: note = X is non-canonical monomer B
CLXLXL

SEQ ID NO: 100 - protein, 6 aa, synthetic construct
  MOD_RES 3: note = X is non-canonical monomer A
  MOD_RES 5: note = X is non-canonical monomer B
CLXLXV

SEQ ID NO: 101 - protein, 7 aa, synthetic construct
  MOD_RES 2: note = X is non-canonical monomer A
  MOD_RES 5: note = X is non-canonical monomer B
CXTTXAA

SEQ ID NO: 102 - protein, 6 aa, synthetic construct
  MOD_RES 3: note = X is non-canonical monomer A
  MOD_RES 5: note = X is non-canonical monomer B
CLXLXL

SEQ ID NO: 103 - protein, 6 aa, synthetic construct
  MOD_RES 3: note = X is non-canonical monomer A
  MOD_RES 5: note = X is non-canonical monomer B
CLXLXV

SEQ ID NO: 104 - protein, 7 aa, synthetic construct
  MOD_RES 2: note = X is non-canonical monomer A
  MOD_RES 5: note = X is non-canonical monomer B
CXTTXAA

SEQ ID NO: 105 - protein, 5 aa, Fusarium sp.
LVLPL

SEQ ID NO: 106 - protein, 7 aa, Chromobacterium sp.
ALTTFAA